(19) United States
(12) Patent Application Publication
(10) Pub. No.: US 2024/0124433 A1
(43) Pub. Date: Apr. 18, 2024

(54) Organic Compound And Organic Light-Emitting Device Comprising Same

(71) Applicant: P&H TECH Co., Ltd, Yongin-si, Gyeonggi-do (KR)

(72) Inventors: Seo-Yong HYUN, Yongin-si, Gyeonggi-do (KR); Gwan-hee PARK, Yongin-si, Gyeonggi-do (KR)

(73) Assignee: P&H TECH Co., Ltd, Yongin-si, Gyeonggi-do (KR)

(21) Appl. No.: 18/256,597

(22) PCT Filed: Sep. 27, 2021
(86) PCT No.: PCT/KR2021/013092

§ 371 Date: Jun. 8, 2023

(30) Foreign Application Priority Data
Dec. 8, 2020 (KR) 10-2020-0170000

(51) Int. Cl. C07D 413/14; H10K 50/16; H10K 50/17; H10K 85/60

(52) U.S. Cl. CPC C07D 413/14; H10K 50/16; H10K 50/171; H10K 85/656

(57) ABSTRACT

The present invention relates to an organic compound and an organic light-emitting device comprising same, the organic compound being employed in an organic layer, such as an electron-transporting layer, in an organic light-emitting device and in a luminous efficiency-improving layer (a capping layer) provided in an organic light-emitting device to achieve light-emitting characteristics such as superior luminous efficiency and low-voltage driving of a device. The present invention can realize improved device characteristics such as superior luminous efficiency and low-voltage driving and thus can be industrially usefully employed in various lighting and display devices.

### TECHNICAL FIELD

The present invention relates to an organic compound, and more particularly, to an organic compound that is employed as a material for a light efficiency improving layer (capping layer) provided in an organic light emitting device, and an organic light emitting device that employs the same, thus achieving greatly improved luminescent properties such as low-voltage driving of the device and excellent luminous efficiency.

### BACKGROUND ART

The organic light emitting device may be formed even on a transparent substrate, and may be driven at a low voltage of 10 V or less compared to a plasma display panel or an inorganic electroluminescence (EL) display. In addition, the device consumes relatively little power and has good color representation. The device may display three colors of green, blue, and read, and thus has recently become a subject of intense interest as a next-generation display device.

However, in order for such an organic light emitting device to exhibit the aforementioned characteristics, the materials constituting an organic layer in the device, such as hole injecting materials, hole transport materials, light emitting materials, electron transport materials, and electron injecting materials, are prerequisites for the support by stable and efficient materials. However, the development of a stable and efficient organic layer material for an organic light emitting device has not yet been sufficiently made.

Thus, further improvements in terms of efficiency and life characteristics are required for good stability, high efficiency, long lifetime, and large size of organic light emitting devices. Particularly, there is a strong need to develop materials constituting each organic layer of organic light emitting devices.

In addition, recently, research aimed at improving the characteristics of organic light emitting devices by changes in the performance of each organic layer material, as well as a technique for improving the color purity and enhancing the luminous efficiency by optimizing the optical thickness between an anode and a cathode are considered as one of the crucial factors for improving the device performance. As an example of this method, an increase in light efficiency and excellent color purity are achieved by using a capping layer on an electrode.

### DISCLOSURE

### Technical Problem

Thus, the present invention has been made in an effort to provide a novel organic compound which may be employed in a light efficiency improving layer (capping layer) provided in an organic layer in an organic light-emitting device or in an organic light-emitting device to implement excellent luminescent properties such as low-voltage driving of the device and improved luminous efficiency, and an organic light-emitting device including the same.

### Technical Solution

In order to solve the problem, the present invention provides an organic compound represented by Formula I below:

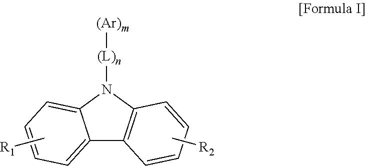

Characteristic structures of Formula I above, a compound implemented by the same, Ar, L, and R₁ to R₂ will be described later.

### Advantageous Effects

When the organic compound according to the present invention is employed as a material for a light efficiency improving layer provided in an organic layer in an organic light emitting device or in an organic light emitting device, the organic compound can be usefully used for various display devices because it is possible to implement luminescent properties such as low-voltage driving of the device and excellent luminous efficiency.

### BEST MODE

Hereinafter, the present invention will be described in more detail. The present invention relates to an organic compound represented by [Formula I] below in an organic light-emitting device, which is capable of obtaining luminescent properties such as low-voltage driving of the device and excellent luminous efficiency.

Structurally, in a skeletal structure represented by Formula I below, it is characterized in that (1) an aryl derivative having one or more cyano groups (CN) needs to be introduced into the N-end of carbazole and (2) benzoxazole and/or benzothiazole derivatives are introduced into the 1- to 4-positions (R₂) of carbazole and the 5- to 8-positions (R₁) of carbazole, and through such structural characteristics, low-voltage driving properties and luminous efficiency properties of the organic light-emitting device can be improved.

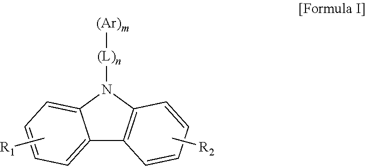

In Formula I above,

Ar is a substituted or unsubstituted aryl group having 6 to 30 carbon atoms, in which at least one or more cyano groups (CN) are substituted, m is an integer of 1 or 2, and when m is 2, a plurality of Ar's are the same as or different from each other.

L is a single bond or a substituted or unsubstituted arylene group having 6 to 30 carbon atoms, n is an integer from 0 to 2, and when n is 2, a plurality of L's are the same as or different from each other.

The compound according to the present invention is characterized in that in the skeleton of Formula I, an aryl structure (-(L)ₙ—(Ar)ₘ) having one or more cyano groups (CN) needs to be introduced into the N-end of carbazole.

R₁ to R₂ are each independently represented by Structural Formula 1 below.

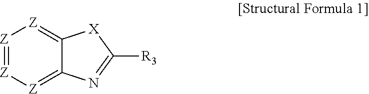

In Structural Formula 1,

X is O or S, Z is CR, and a plurality of R's are the same as or different from each other.

R and R₃ are each independently selected from among hydrogen, deuterium, a halogen group, a cyano group, a substituted or unsubstituted alkyl group having 1 to 20 carbon atoms, a substituted or unsubstituted alkenyl group having 2 to 20 carbon atoms, a substituted or unsubstituted cycloalkyl group having 3 to 20 carbon atoms, a substituted or unsubstituted alkoxy group having 1 to 20 carbon atoms, a substituted or unsubstituted halogenated alkyl group having 1 to 20 carbon atoms, a substituted or unsubstituted halogenated alkoxy group having 1 to 20 carbon atoms, a substituted or unsubstituted aryl group having 6 to 30 carbon atoms and a substituted or unsubstituted heteroaryl group having 3 to 30 carbon atoms.

Any one of the plurality of R's and R₃ is a moiety in which Structural Formula 1 above is each linked to Formula I above at the R₁ and R₂ positions.

Further, the plurality of R's and R₃ may be bonded to each other or linked to an adjacent substituent to form an aromatic monocyclic or polycyclic ring, and accordingly, Structural Formula 1 above may be any one selected among structural formulae represented by Structural Formula 2 to Structural Formulae 6 below.

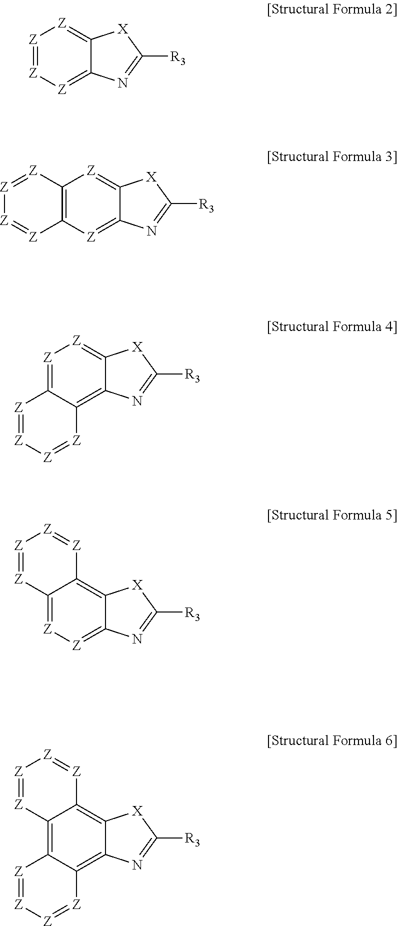

In Structural Formulae 2 to 6 above,

X is O or S, Z is CR, and a plurality of R's are the same as or different from each other.

R and R₃ are each independently selected from among hydrogen, deuterium, a halogen group, a cyano group, a substituted or unsubstituted alkyl group having 1 to 20 carbon atoms, a substituted or unsubstituted alkenyl group having 2 to 20 carbon atoms, a substituted or unsubstituted cycloalkyl group having 3 to 20 carbon atoms, a substituted or unsubstituted alkoxy group having 1 to 20 carbon atoms, a substituted or unsubstituted halogenated alkyl group having 1 to 20 carbon atoms, a substituted or unsubstituted halogenated alkoxy group having 1 to 20 carbon atoms, a substituted or unsubstituted aryl group having 6 to 30 carbon atoms and a substituted or unsubstituted heteroaryl group having 3 to 30 carbon atoms.

Any one of the plurality of R's and R₃ is a moiety in which Structural Formula 1 above is each linked to Formula I above at the R₁ and R₂ positions.

Meanwhile, in the definitions of Ar, L, R and R₃, the ‘substituted or unsubstituted’ means being substituted with one or two or more substituents selected from the group consisting of hydrogen, deuterium, a halogen group, a cyano group, a nitro group, a hydroxyl group, a silyl group, an alkyl group, a halogenated alkyl group, a deuterated alkyl group, a cycloalkyl group, a heterocycloalkyl group, an alkoxy group, a halogenated alkoxy group, a deuterated alkoxy group, an aryl group, a heteroaryl group, an alkylamine group, an arylamine group and a silyl group, being substituted with a substituent to which two or more substituent among the above-described substituents are linked, or having no substituent.

For specific examples, the substituted arylene group means that a phenyl group, a biphenyl group, a naphthalene group, a fluorenyl group, a pyrenyl group, a phenanthrenyl group, a perylene group, a tetracenyl group, and an anthracenyl group are substituted with other substituents.

In addition, the substituted heteroaryl group means that a pyridyl group, a thiophenyl group, a triazine group, a quinoline group, a phenanthroline group, an imidazole group, a thiazole group, an oxazole group, a carbazole group and a condensate heteroring group thereof, for example, a benzquinoline group, a benzimidazole group, a benzoxazole group, a benzthiazole group, a benzcarbazole group, a dibenzothiophenyl group, and a dibenzofuran group are substituted with other substituents.

In an embodiment of the present invention, examples of the substituents will be described in detail below, but are not limited thereto.

In an embodiment of the present invention, the alkyl groups may be straight or branched. The number of carbon atoms in the alkyl groups is not particularly limited but is preferably from 1 to 20. Specific examples of the alkyl groups include, but are not limited to, methyl, ethyl, propyl, n-propyl, isopropyl, butyl, n-butyl, isobutyl, tert-butyl, sec-butyl, 1-methylbutyl, 1-ethylbutyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohexylmethyl, octyl, n-octyl, tert-octyl, 1-methylheptyl, 2-ethylhexyl, 2-propylpentyl, n-nonyl, 2,2-dimethylheptyl, 1-ethylpropyl, 1,1-dimethylpropyl, isohexyl, 2-methylpentyl, 4-methylhexyl, and 5-methylhexyl groups.

In an embodiment of the present invention, the alkoxy groups may be straight or branched. The number of carbon atoms in the alkoxy groups is not particularly limited but is preferably from 1 to 20 as long as steric hindrance is avoided. Specific examples of the alkoxy groups include, but are not limited to, methoxy, ethoxy, n-propoxy, isopropoxy, i-propyloxy, n-butoxy, isobutoxy, tert-butoxy, sec-butoxy, n-pentyloxy, neopentyloxy, isopentyloxy, n-hexyloxy, 3,3-dimethylbutyloxy, 2-ethylbutyl oxy, n-octyloxy, n-nonyloxy, n-decyloxy, benzyloxy, and p-methylbenzyloxy groups.

In an embodiment of the present invention, the deuterated alkyl group or alkoxy group and the halogenated alkyl group or alkoxy group mean an alkyl group or alkoxy group in which the above alkyl group or alkoxy group is substituted with deuterium or a halogen group.

In an embodiment of the present invention, the aryl groups may be monocyclic or polycyclic. The number of carbon atoms in the aryl groups is not particularly limited but is preferably from 6 to 30. Examples of the monocyclic aryl groups include phenyl, biphenyl, terphenyl, and stilbene groups but the scope of the present invention is not limited thereto. Examples of the polycyclic aryl groups include naphthyl, anthracenyl, phenanthrenyl, pyrenyl, perylenyl, tetracenyl, chrysenyl, fluorenyl, acenaphathcenyl, triphenylene, and fluoranthrene groups, but the scope of the present invention is not limited thereto.

In addition, in an embodiment of the present invention, the fluorenyl groups refer to structures in which two cyclic organic compounds are linked through one atom, and examples thereof include

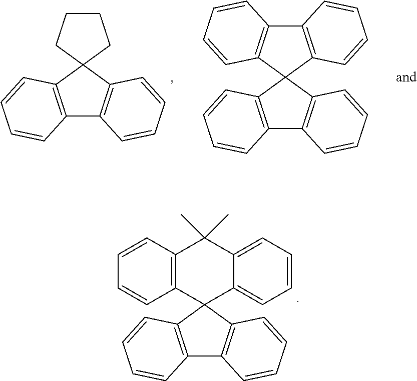

In an embodiment of the present invention, the fluorenyl groups include open structures in which one of the two cyclic organic compounds linked through one atom is cleaved, and examples thereof include

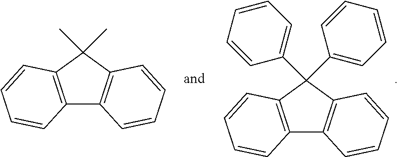

In addition, carbon atoms of the ring may be substituted with any one or more heteroatoms selected from among N, S and O, and examples thereof include

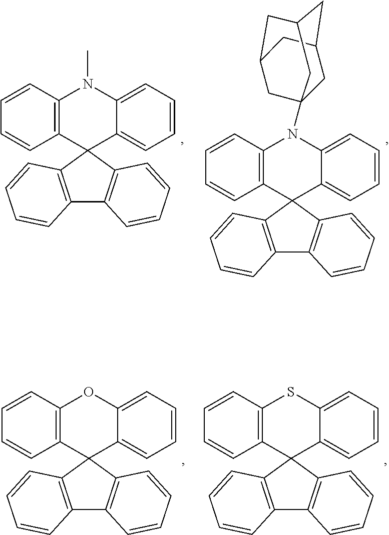

and the like.

In an embodiment of the present invention, the heteroaryl groups refer to heterocyclic groups containing heteroatoms selected from O, N, and S. The number of carbon atoms is not particularly limited, but preferably from 2 to 30. In an embodiment of the present invention, specific examples thereof include, but are not limited to, thiophene, furan, pyrrole, imidazole, thiazole, oxazole, oxadiazole, triazole, pyridyl, bipyridyl, pyrimidyl, triazine, triazole, acridyl, pyridazine, pyrazinyl, quinolinyl, quinazoline, quinoxalinyl, phthalazinyl, pyridopyrimidinyl, pyridopyrazinyl, pyrazinopyrazinyl, isoquinoline, indole, carbazole, benzoxazole, benzimidazole, benzothiazole, benzocarbazole, benzothiophene, dibenzothiophene, benzofuranyl, dibenzofuranyl, phenanthroline, thiazolyl, isoxazolyl, oxadiazolyl, thiadiazolyl, benzothiazolyl, phenothiazinyl, phenoxazine, and phenothiazine groups.

In an embodiment of the present invention, the silyl group is an unsubstituted silyl group or a silyl group substituted with an alkyl group, an aryl group, and the like, and specific examples of the silyl group include trimethylsilyl, triethylsilyl, triphenylsilyl, trimethoxysilyl, dimethoxyphenylsilyl, diphenylmethylsilyl, diphenylvinylsilyl, methylcyclobutylsilyl, dimethylfurylsilyl, and the like, but are not limited thereto.

In an embodiment of the present invention, the amine group may be —NH₂, an alkylamine group, an arylamine group, and the like, the arylamine group means an amine substituted with aryl, the alkylamine group means an amine substituted with alkyl, examples of the arylamine group include a substituted or unsubstituted monoarylamine group, a substituted or unsubstituted diarylamine group, or a substituted or unsubstituted triarylamine group, the aryl group in the arylamine group is the same as the definition of the aryl group, and the alkyl group of the alkylamine group is also the same as the definition of the alkyl group.

Specific examples of the halogen groups as substituents used in an embodiment of the present invention include fluorine (F), chlorine (Cl), and bromine (Br).

In an embodiment of the present invention, a cycloalkyl group refers to a monocyclic, polycyclic and spiro alkyl radical, includes the same, and preferably contains a cyclic carbon atom having 3 to 20 carbon atoms, and includes cyclopropyl, cyclopentyl, cyclohexyl, bicycloheptyl, spirodecyl, spiroundecyl, adamantyl, and the like, and the cycloalkyl group may be arbitrarily substituted.

In an embodiment of the present invention, the heterocycloalkyl group refers to an aromatic or non-aromatic cyclic radical containing one or more heteroatoms, and includes the same, and one or more heteroatoms are selected from among O, S, N, P, B, Si, and Se, preferably O, N or S, and specifically, in the case of including N, the one or more heteroatoms may be aziridine, pyrrolidine, piperidine, azepane, azocane, and the like.

The organic compound represented by Formula I above according to the present invention may be used for various organic layers including an electron transport layer in an organic light-emitting device due to its structural specificity, and may also be used as a material for a light efficiency improving layer provided in the organic light-emitting device.

Preferred and specific examples of the organic compound represented by Formula I according to an embodiment of the present invention include the following compounds, but are not limited thereto:

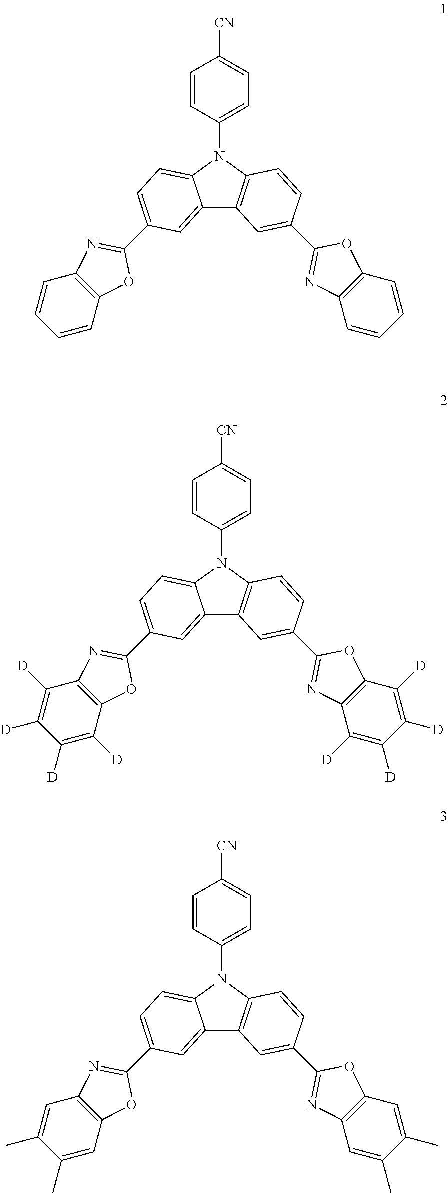

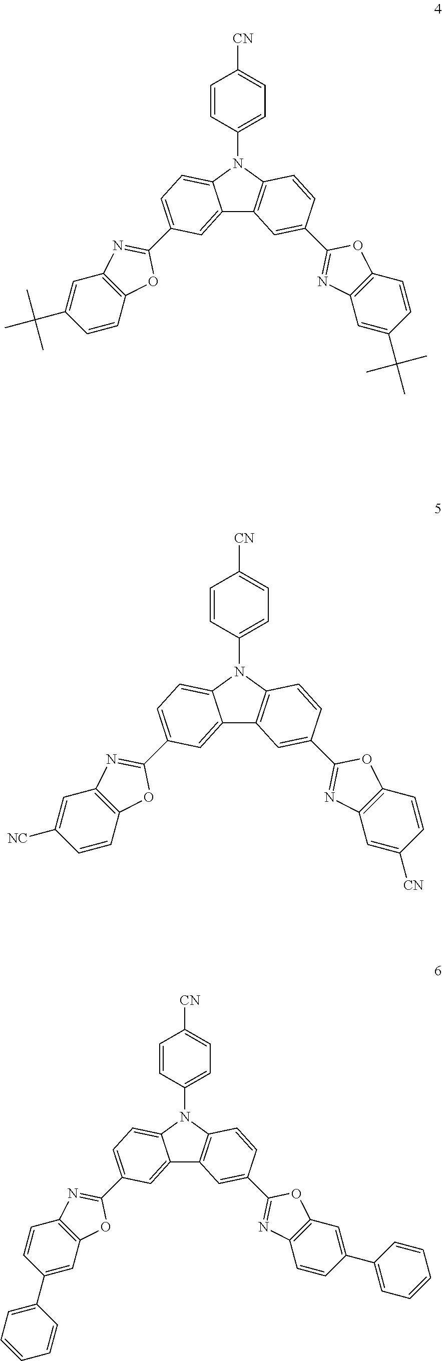 [SMILES: CC(C)(C)c1ccc2oc(-c3ccc4c(c3)c3cc(-c5nc6cc(C(C)(C)C)ccc6o5)ccc3n4-c3ccc(C#N)cc3)nc2c1 ; N#Cc1ccc(-n2c3ccc(-c4nc5cc(C#N)ccc5o4)cc3c3cc(-c4nc5cc(C#N)ccc5o4)ccc32)cc1 ; N#Cc1ccc(-n2c3ccc(-c4nc5ccc(-c6ccccc6)cc5o4)cc3c3cc(-c4nc5ccc(-c6ccccc6)cc5o4)ccc32)cc1]

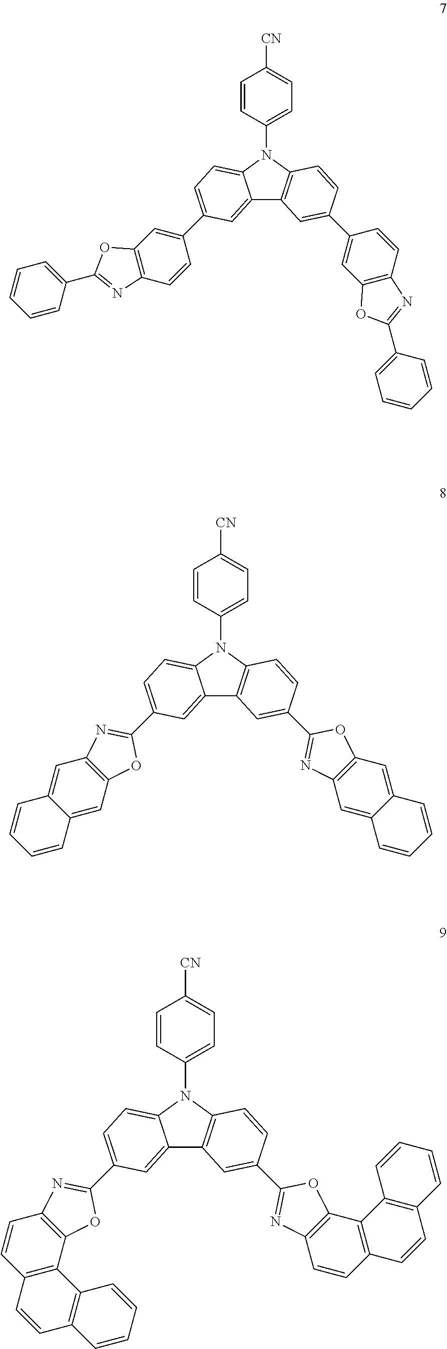 [SMILES: N#Cc1ccc(-n2c3ccc(-c4ccc5nc(-c6ccccc6)oc5c4)cc3c3cc(-c4ccc5nc(-c6ccccc6)oc5c4)ccc32)cc1 ; N#Cc1ccc(-n2c3ccc(-c4nc5cc6ccccc6cc5o4)cc3c3cc(-c4nc5cc6ccccc6cc5o4)ccc32)cc1 ; N#Cc1ccc(-n2c3ccc(-c4nc5ccc6ccc7ccccc7c6c5o4)cc3c3cc(-c4nc5ccc6ccc7ccccc7c6c5o4)ccc32)cc1]

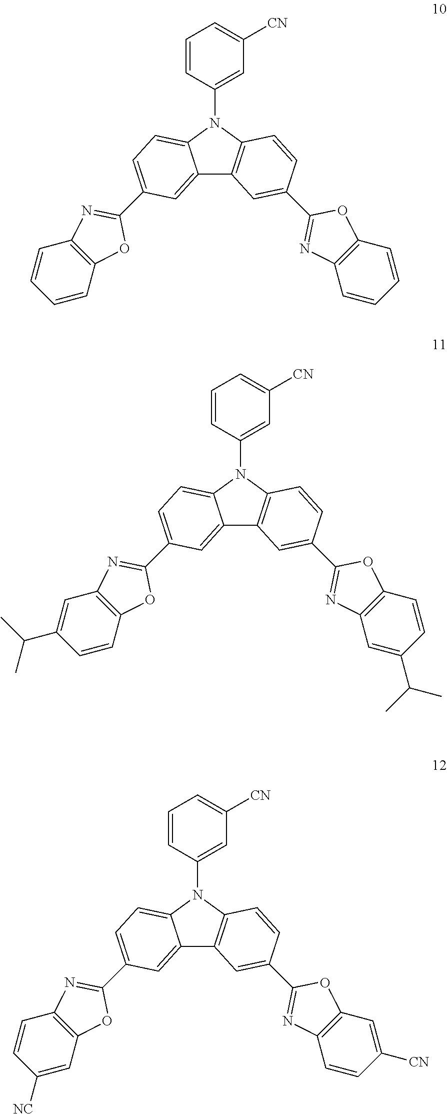 [SMILES: N#Cc1cccc(-n2c3ccc(-c4nc5ccccc5o4)cc3c3cc(-c4nc5ccccc5o4)ccc32)c1 ; CC(C)c1ccc2oc(-c3ccc4c(c3)c3cc(-c5nc6cc(C(C)C)ccc6o5)ccc3n4-c3cccc(C#N)c3)nc2c1 ; N#Cc1cccc(-n2c3ccc(-c4nc5ccc(C#N)cc5o4)cc3c3cc(-c4nc5ccc(C#N)cc5o4)ccc32)c1]

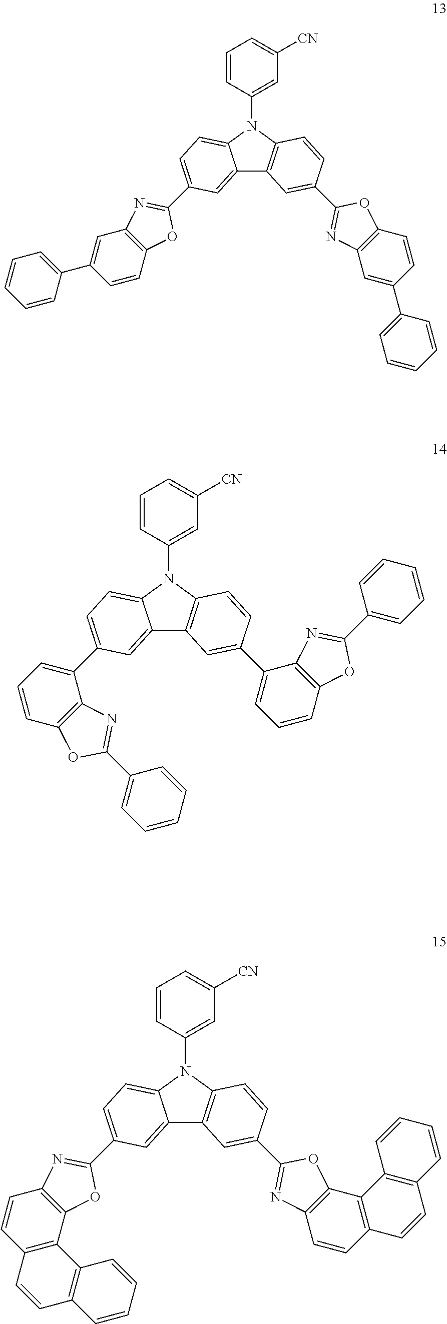 [SMILES: N#Cc1cccc(-n2c3ccc(-c4nc5cc(-c6ccccc6)ccc5o4)cc3c3cc(-c4nc5cc(-c6ccccc6)ccc5o4)ccc32)c1 ; N#Cc1cccc(-n2c3ccc(-c4cccc5oc(-c6ccccc6)nc45)cc3c3cc(-c4cccc5oc(-c6ccccc6)nc45)ccc32)c1 ; N#Cc1cccc(-n2c3ccc(-c4nc5ccc6ccc7ccccc7c6c5o4)cc3c3cc(-c4nc5ccc6ccc7ccccc7c6c5o4)ccc32)c1]

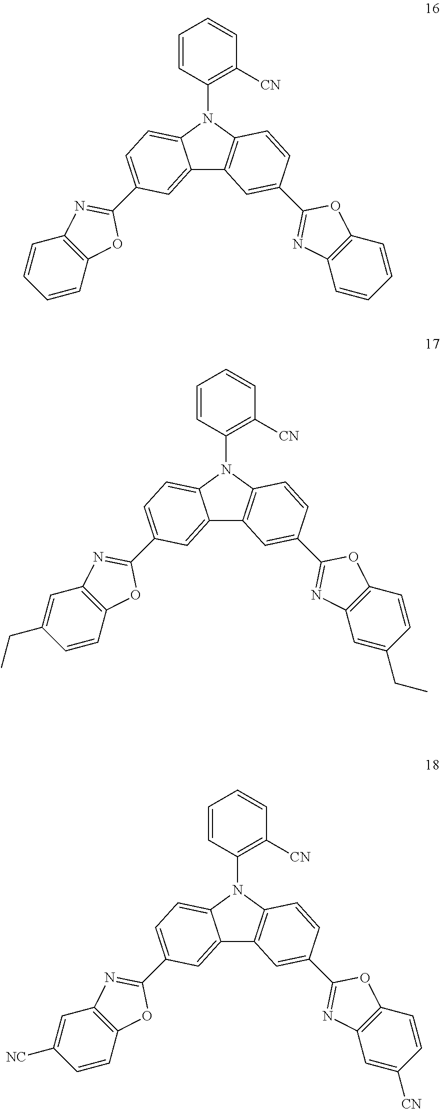 [SMILES: N#Cc1ccccc1-n1c2ccc(-c3nc4ccccc4o3)cc2c2cc(-c3nc4ccccc4o3)ccc21 ; CCc1ccc2oc(-c3ccc4c(c3)c3cc(-c5nc6cc(CC)ccc6o5)ccc3n4-c3ccccc3C#N)nc2c1 ; N#Cc1ccc2oc(-c3ccc4c(c3)c3cc(-c5nc6cc(C#N)ccc6o5)ccc3n4-c3ccccc3C#N)nc2c1]

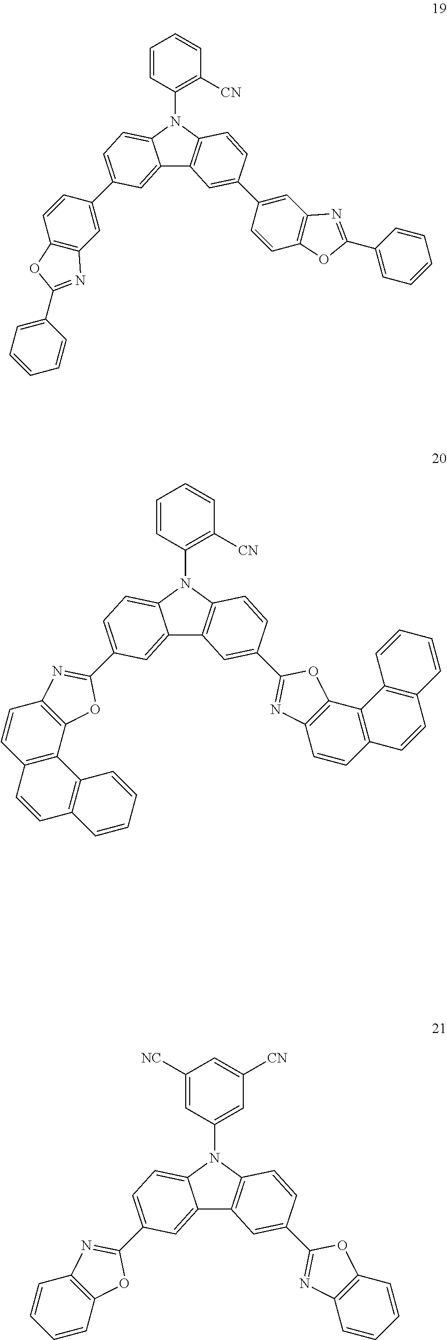 [SMILES: N#Cc1ccccc1-n1c2ccc(-c3ccc4oc(-c5ccccc5)nc4c3)cc2c2cc(-c3ccc4oc(-c5ccccc5)nc4c3)ccc21 ; N#Cc1ccccc1-n1c2ccc(-c3nc4ccc5ccc6ccccc6c5c4o3)cc2c2cc(-c3nc4ccc5ccc6ccccc6c5c4o3)ccc21 ; N#Cc1cc(C#N)cc(-n2c3ccc(-c4nc5ccccc5o4)cc3c3cc(-c4nc5ccccc5o4)ccc32)c1]

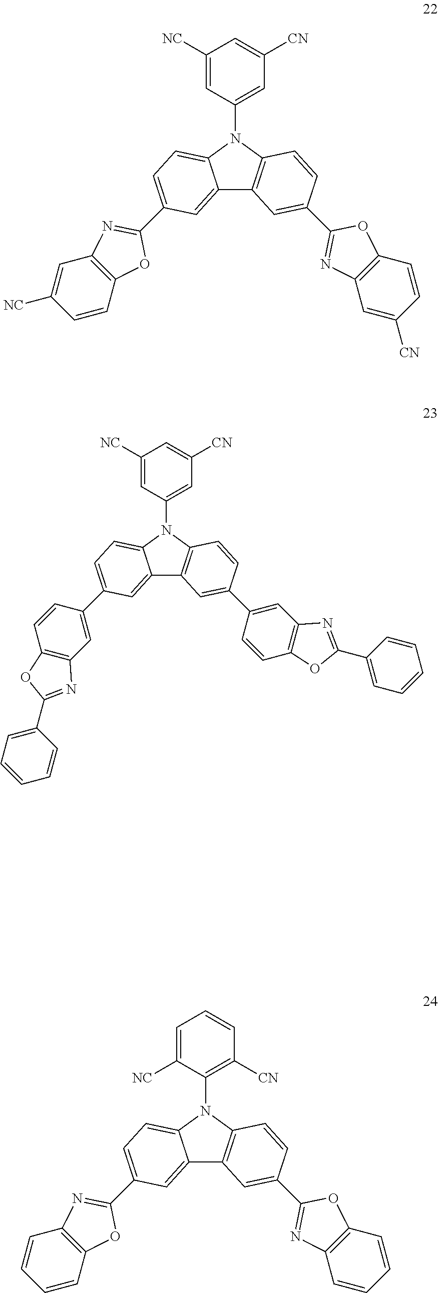 [SMILES: N#Cc1cc(C#N)cc(-n2c3ccc(-c4nc5cc(C#N)ccc5o4)cc3c3cc(-c4nc5cc(C#N)ccc5o4)ccc32)c1 ; N#Cc1cc(C#N)cc(-n2c3ccc(-c4ccc5oc(-c6ccccc6)nc5c4)cc3c3cc(-c4ccc5oc(-c6ccccc6)nc5c4)ccc32)c1 ; N#Cc1cccc(C#N)c1-n1c2ccc(-c3nc4ccccc4o3)cc2c2cc(-c3nc4ccccc4o3)ccc21]

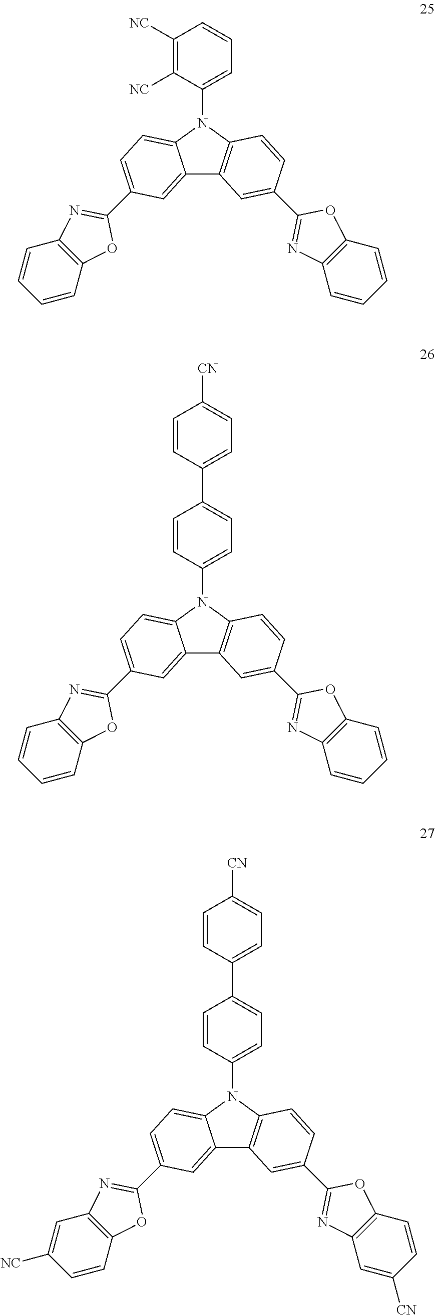 [SMILES: N#Cc1cccc(-n2c3ccc(-c4nc5ccccc5o4)cc3c3cc(-c4nc5ccccc5o4)ccc32)c1C#N ; N#Cc1ccc(-c2ccc(-n3c4ccc(-c5nc6ccccc6o5)cc4c4cc(-c5nc6ccccc6o5)ccc43)cc2)cc1 ; N#Cc1ccc(-c2ccc(-n3c4ccc(-c5nc6cc(C#N)ccc6o5)cc4c4cc(-c5nc6cc(C#N)ccc6o5)ccc43)cc2)cc1]

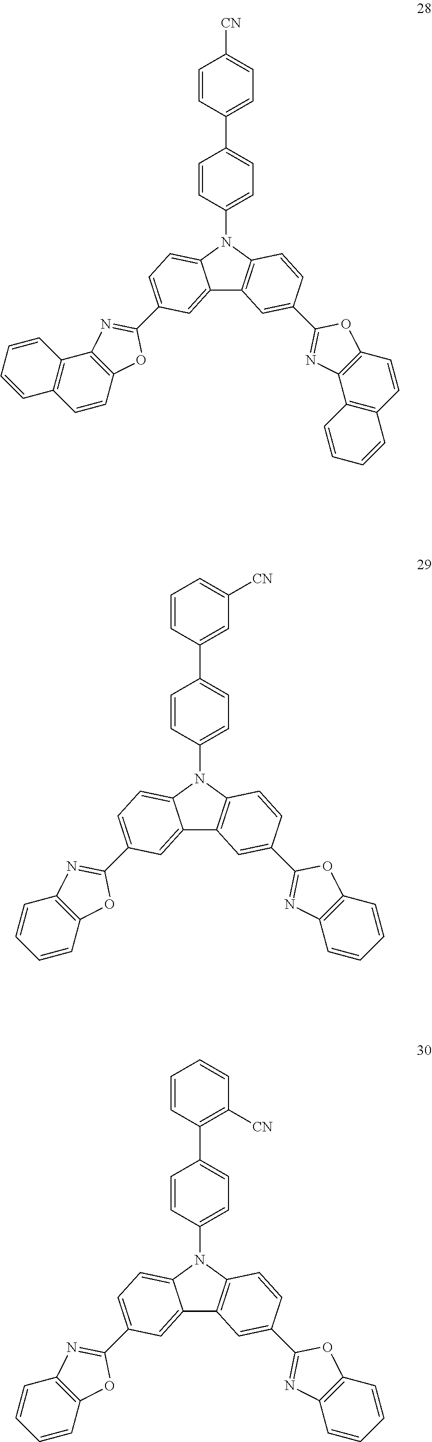 [SMILES: N#Cc1ccc(-c2ccc(-n3c4ccc(-c5nc6c(ccc7ccccc76)o5)cc4c4cc(-c5nc6c(ccc7ccccc76)o5)ccc43)cc2)cc1 ; N#Cc1cccc(-c2ccc(-n3c4ccc(-c5nc6ccccc6o5)cc4c4cc(-c5nc6ccccc6o5)ccc43)cc2)c1 ; N#Cc1ccccc1-c1ccc(-n2c3ccc(-c4nc5ccccc5o4)cc3c3cc(-c4nc5ccccc5o4)ccc32)cc1]

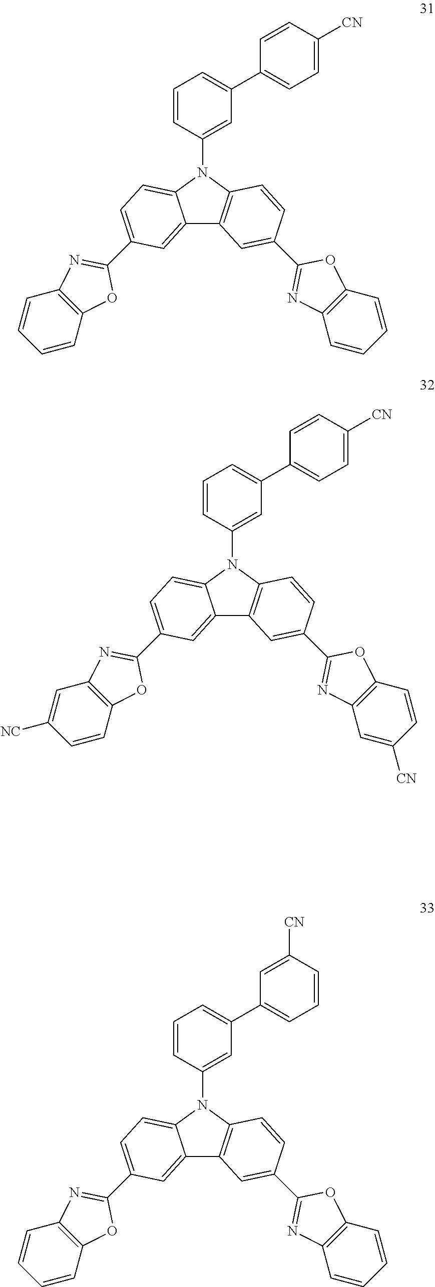 [SMILES: N#Cc1ccc(-c2cccc(-n3c4ccc(-c5nc6ccccc6o5)cc4c4cc(-c5nc6ccccc6o5)ccc43)c2)cc1 ; N#Cc1ccc(-c2cccc(-n3c4ccc(-c5nc6cc(C#N)ccc6o5)cc4c4cc(-c5nc6cc(C#N)ccc6o5)ccc43)c2)cc1 ; N#Cc1cccc(-c2cccc(-n3c4ccc(-c5nc6ccccc6o5)cc4c4cc(-c5nc6ccccc6o5)ccc43)c2)c1]

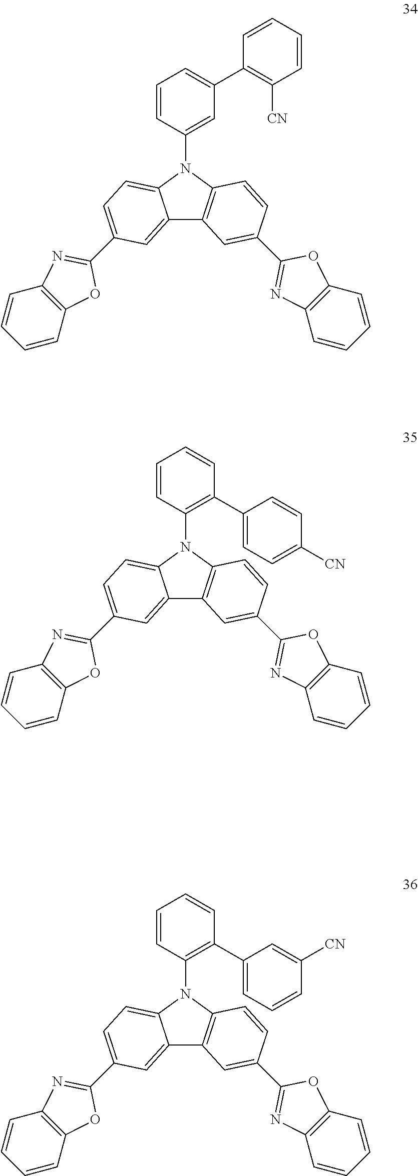 [SMILES: N#Cc1ccccc1-c1cccc(-n2c3ccc(-c4nc5ccccc5o4)cc3c3cc(-c4nc5ccccc5o4)ccc32)c1 ; N#Cc1ccc(-c2ccccc2-n2c3ccc(-c4nc5ccccc5o4)cc3c3cc(-c4nc5ccccc5o4)ccc32)cc1 ; N#Cc1cccc(-c2ccccc2-n2c3ccc(-c4nc5ccccc5o4)cc3c3cc(-c4nc5ccccc5o4)ccc32)c1]

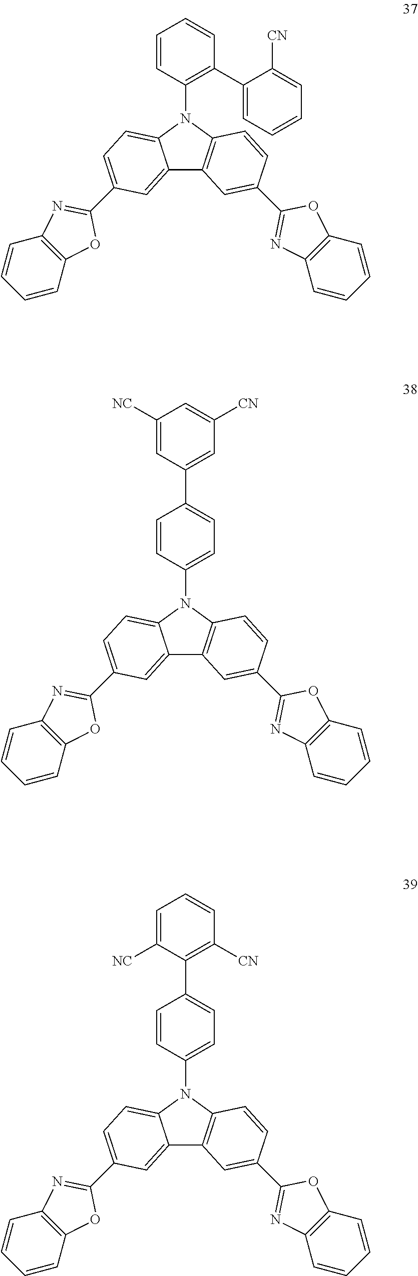 [SMILES: N#Cc1ccccc1-c1ccccc1-n1c2ccc(-c3nc4ccccc4o3)cc2c2cc(-c3nc4ccccc4o3)ccc21 ; N#Cc1cc(C#N)cc(-c2ccc(-n3c4ccc(-c5nc6ccccc6o5)cc4c4cc(-c5nc6ccccc6o5)ccc43)cc2)c1 ; N#Cc1cccc(C#N)c1-c1ccc(-n2c3ccc(-c4nc5ccccc5o4)cc3c3cc(-c4nc5ccccc5o4)ccc32)cc1]

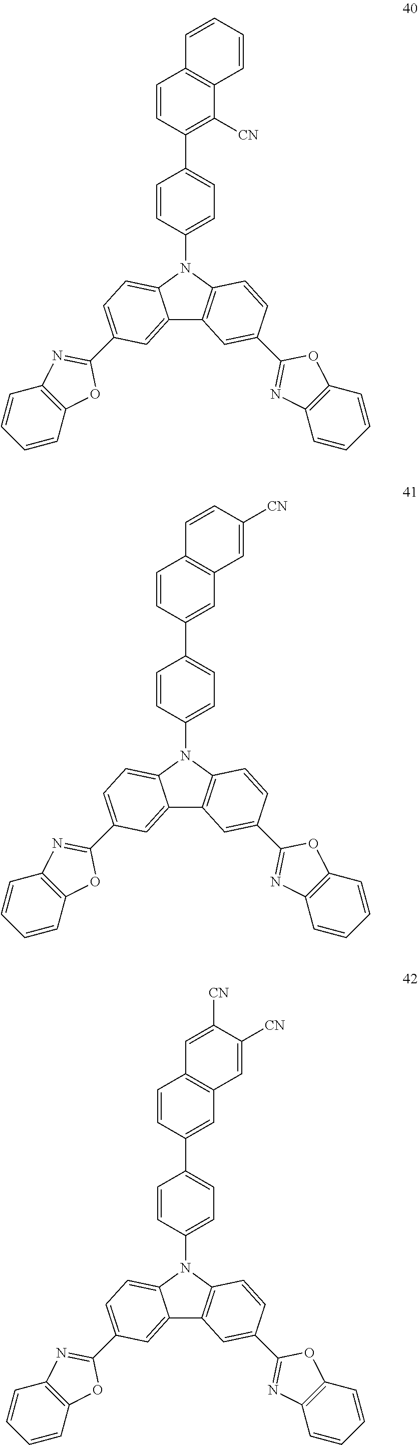 [SMILES: N#Cc1c(-c2ccc(-n3c4ccc(-c5nc6ccccc6o5)cc4c4cc(-c5nc6ccccc6o5)ccc43)cc2)ccc2ccccc12 ; N#Cc1ccc2ccc(-c3ccc(-n4c5ccc(-c6nc7ccccc7o6)cc5c5cc(-c6nc7ccccc7o6)ccc54)cc3)cc2c1 ; N#Cc1cc2ccc(-c3ccc(-n4c5ccc(-c6nc7ccccc7o6)cc5c5cc(-c6nc7ccccc7o6)ccc54)cc3)cc2cc1C#N]

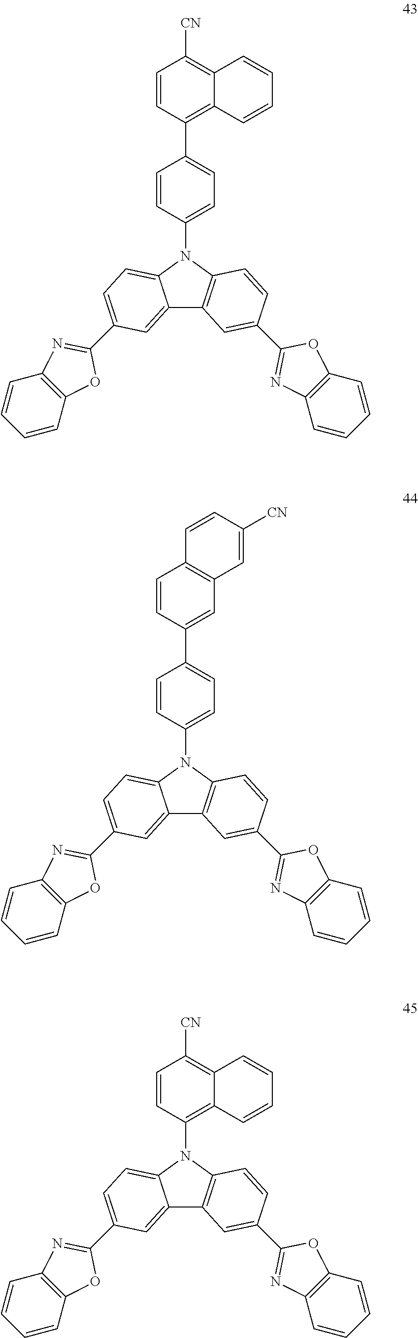 [SMILES: N#Cc1ccc(-c2ccc(-n3c4ccc(-c5nc6ccccc6o5)cc4c4cc(-c5nc6ccccc6o5)ccc43)cc2)c2ccccc12 ; N#Cc1ccc2ccc(-c3ccc(-n4c5ccc(-c6nc7ccccc7o6)cc5c5cc(-c6nc7ccccc7o6)ccc54)cc3)cc2c1 ; N#Cc1ccc(-n2c3ccc(-c4nc5ccccc5o4)cc3c3cc(-c4nc5ccccc5o4)ccc32)c2ccccc12]

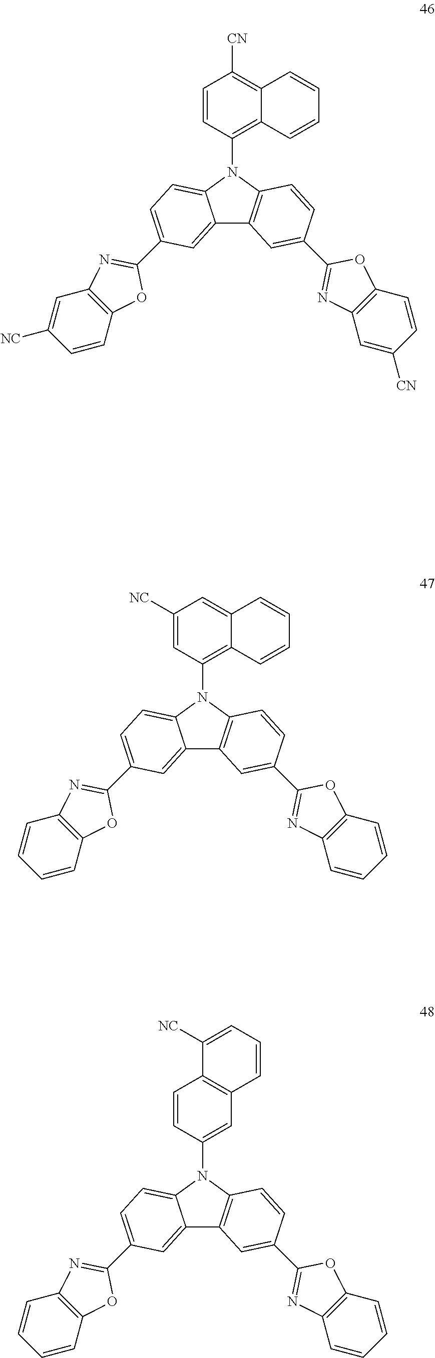 [SMILES: N#Cc1ccc2oc(-c3ccc4c(c3)c3cc(-c5nc6cc(C#N)ccc6o5)ccc3n4-c3ccc(C#N)c4ccccc34)nc2c1 ; N#Cc1cc(-n2c3ccc(-c4nc5ccccc5o4)cc3c3cc(-c4nc5ccccc5o4)ccc32)c2ccccc2c1 ; N#Cc1cccc2cc(-n3c4ccc(-c5nc6ccccc6o5)cc4c4cc(-c5nc6ccccc6o5)ccc43)ccc12]

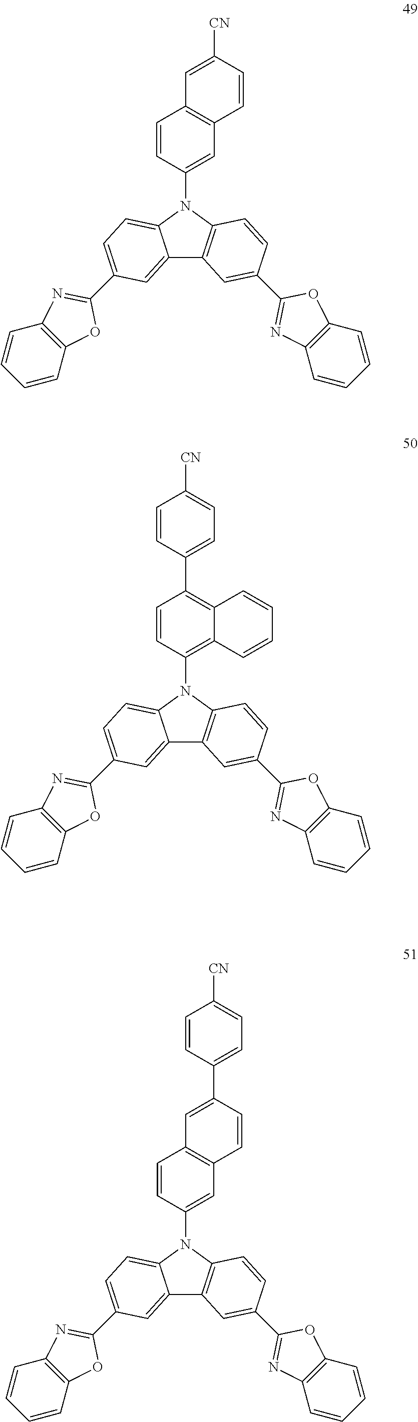 [SMILES: N#Cc1ccc2cc(-n3c4ccc(-c5nc6ccccc6o5)cc4c4cc(-c5nc6ccccc6o5)ccc43)ccc2c1 ; N#Cc1ccc(-c2ccc(-n3c4ccc(-c5nc6ccccc6o5)cc4c4cc(-c5nc6ccccc6o5)ccc43)c3ccccc23)cc1 ; N#Cc1ccc(-c2ccc3cc(-n4c5ccc(-c6nc7ccccc7o6)cc5c5cc(-c6nc7ccccc7o6)ccc54)ccc3c2)cc1]

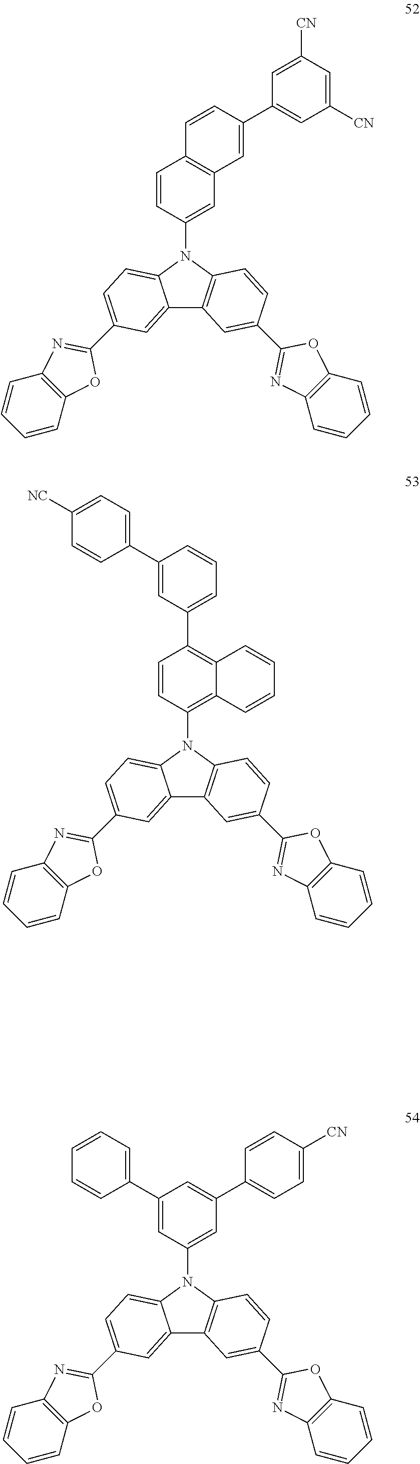 [SMILES: N#Cc1cc(C#N)cc(-c2ccc3ccc(-n4c5ccc(-c6nc7ccccc7o6)cc5c5cc(-c6nc7ccccc7o6)ccc54)cc3c2)c1 ; N#Cc1ccc(-c2cccc(-c3ccc(-n4c5ccc(-c6nc7ccccc7o6)cc5c5cc(-c6nc7ccccc7o6)ccc54)c4ccccc34)c2)cc1 ; N#Cc1ccc(-c2cc(-c3ccccc3)cc(-n3c4ccc(-c5nc6ccccc6o5)cc4c4cc(-c5nc6ccccc6o5)ccc43)c2)cc1]

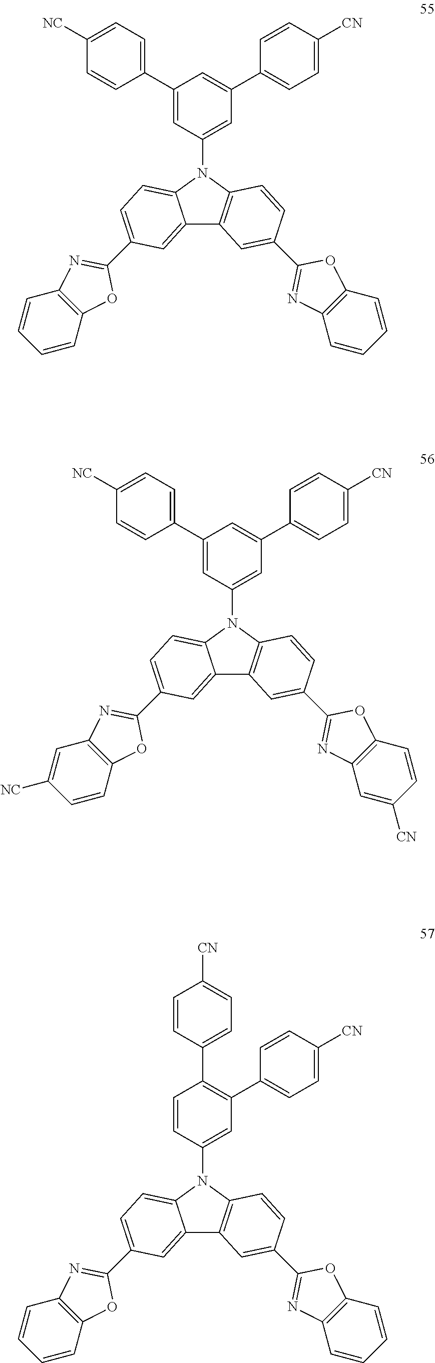 [SMILES: N#Cc1ccc(-c2cc(-c3ccc(C#N)cc3)cc(-n3c4ccc(-c5nc6ccccc6o5)cc4c4cc(-c5nc6ccccc6o5)ccc43)c2)cc1 ; N#Cc1ccc(-c2cc(-c3ccc(C#N)cc3)cc(-n3c4ccc(-c5nc6cc(C#N)ccc6o5)cc4c4cc(-c5nc6cc(C#N)ccc6o5)ccc43)c2)cc1 ; N#Cc1ccc(-c2ccc(-n3c4ccc(-c5nc6ccccc6o5)cc4c4cc(-c5nc6ccccc6o5)ccc43)cc2-c2ccc(C#N)cc2)cc1]

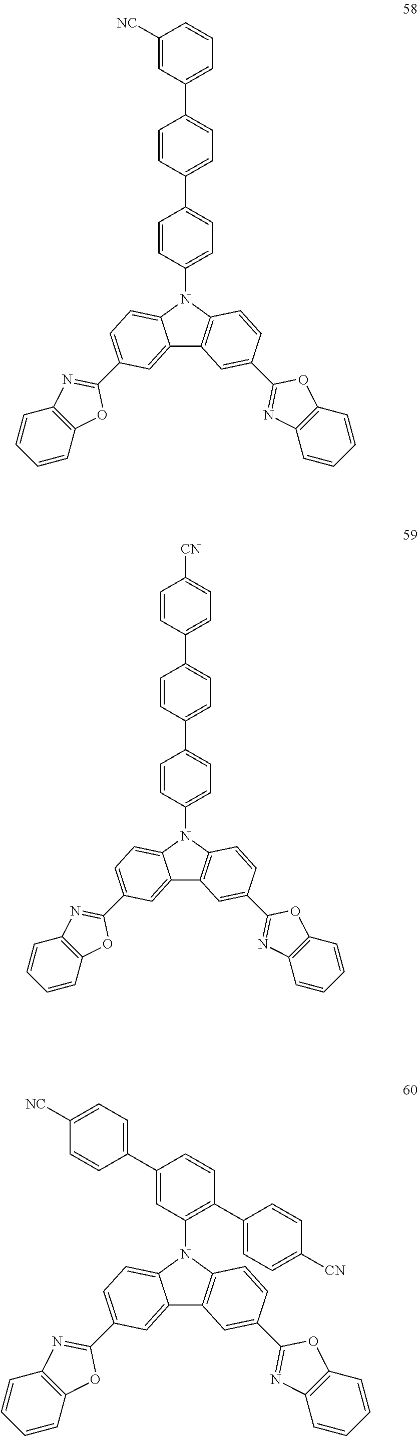 [SMILES: N#Cc1cccc(-c2ccc(-c3ccc(-n4c5ccc(-c6nc7ccccc7o6)cc5c5cc(-c6nc7ccccc7o6)ccc54)cc3)cc2)c1 ; N#Cc1ccc(-c2ccc(-c3ccc(-n4c5ccc(-c6nc7ccccc7o6)cc5c5cc(-c6nc7ccccc7o6)ccc54)cc3)cc2)cc1 ; N#Cc1ccc(-c2ccc(-c3ccc(C#N)cc3)c(-n3c4ccc(-c5nc6ccccc6o5)cc4c4cc(-c5nc6ccccc6o5)ccc43)c2)cc1]

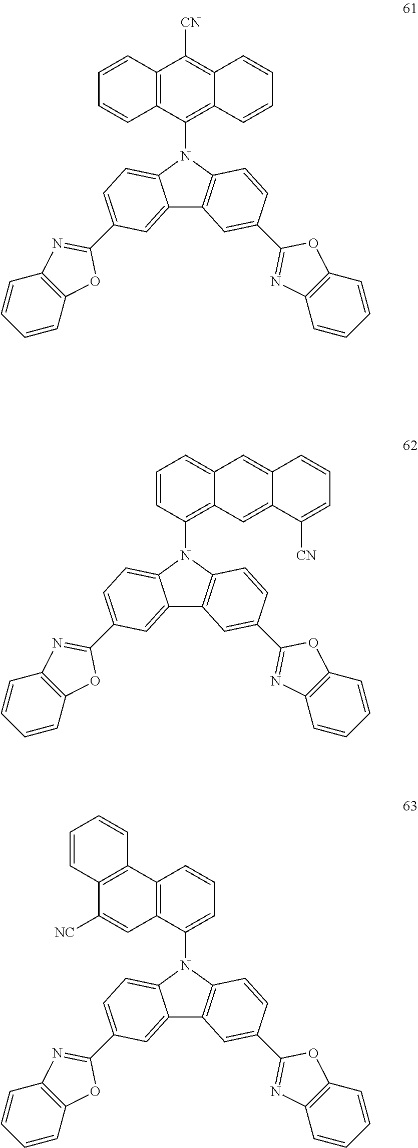 [SMILES: N#Cc1c2ccccc2c(-n2c3ccc(-c4nc5ccccc5o4)cc3c3cc(-c4nc5ccccc5o4)ccc32)c2ccccc12 ; N#Cc1cccc2cc3cccc(-n4c5ccc(-c6nc7ccccc7o6)cc5c5cc(-c6nc7ccccc7o6)ccc54)c3cc12 ; N#Cc1cc2c(-n3c4ccc(-c5nc6ccccc6o5)cc4c4cc(-c5nc6ccccc6o5)ccc43)cccc2c2ccccc12]

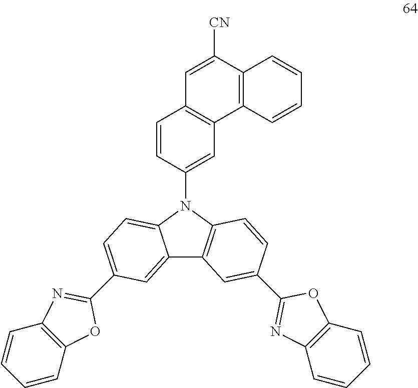 [SMILES: N#Cc1cc2ccc(-n3c4ccc(-c5nc6ccccc6o5)cc4c4cc(-c5nc6ccccc6o5)ccc43)cc2c2ccccc12]

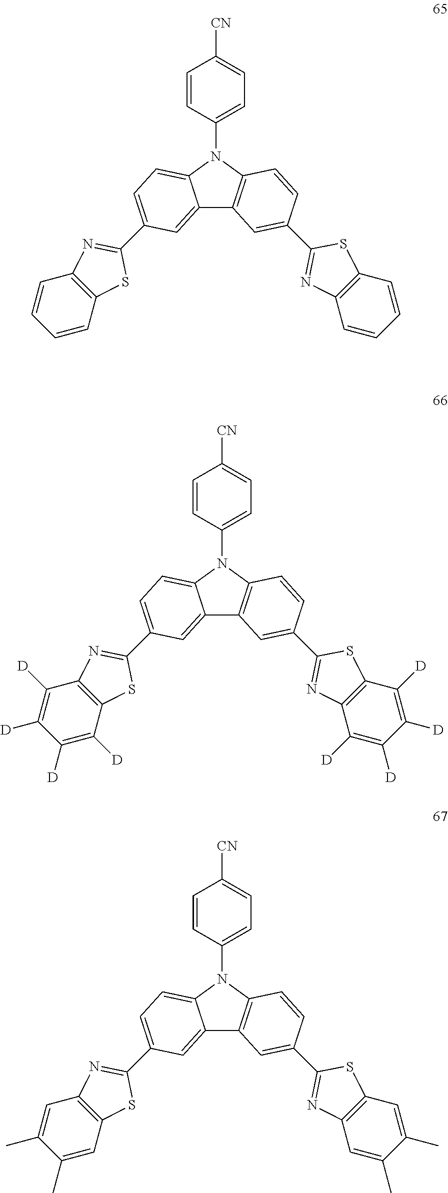

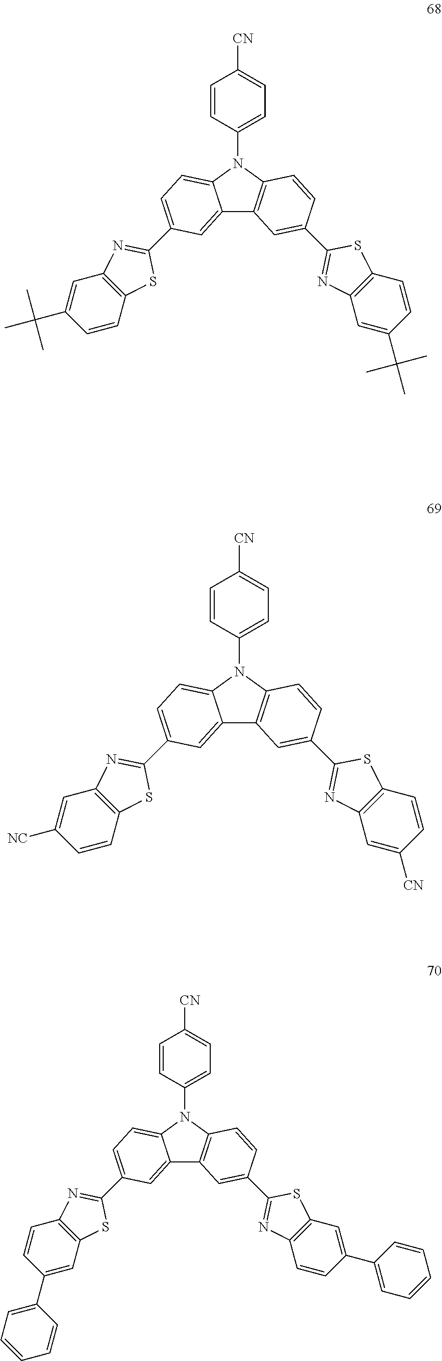 [SMILES: CC(C)(C)c1ccc2sc(-c3ccc4c(c3)c3cc(-c5nc6cc(C(C)(C)C)ccc6s5)ccc3n4-c3ccc(C#N)cc3)nc2c1 ; N#Cc1ccc(-n2c3ccc(-c4nc5cc(C#N)ccc5s4)cc3c3cc(-c4nc5cc(C#N)ccc5s4)ccc32)cc1 ; N#Cc1ccc(-n2c3ccc(-c4nc5ccc(-c6ccccc6)cc5s4)cc3c3cc(-c4nc5ccc(-c6ccccc6)cc5s4)ccc32)cc1]

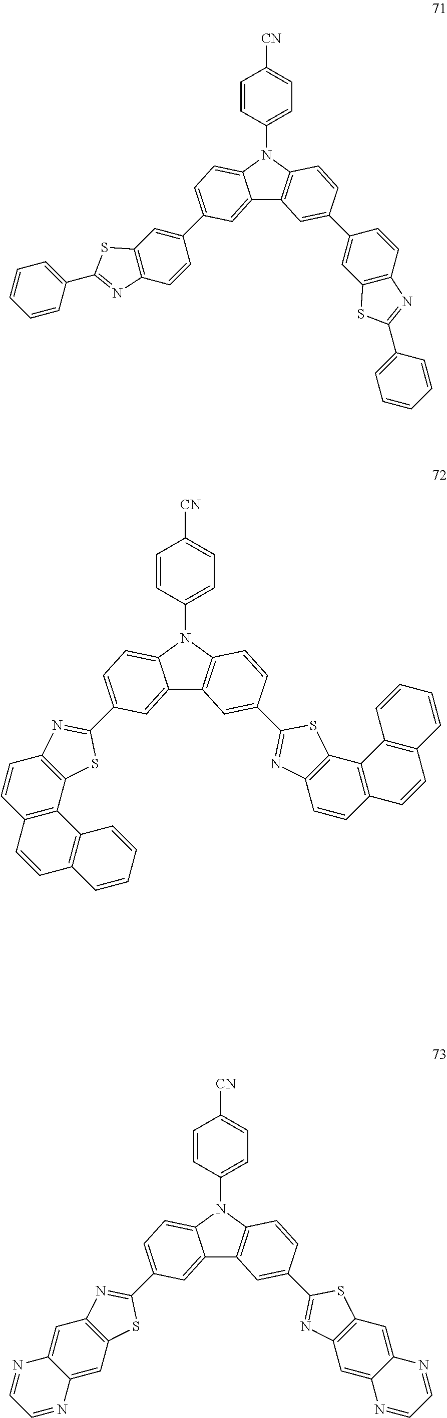 [SMILES: N#Cc1ccc(-n2c3ccc(-c4ccc5nc(-c6ccccc6)sc5c4)cc3c3cc(-c4ccc5nc(-c6ccccc6)sc5c4)ccc32)cc1 ; N#Cc1ccc(-n2c3ccc(-c4nc5ccc6ccc7ccccc7c6c5s4)cc3c3cc(-c4nc5ccc6ccc7ccccc7c6c5s4)ccc32)cc1 ; N#Cc1ccc(-n2c3ccc(-c4nc5cc6nccnc6cc5s4)cc3c3cc(-c4nc5cc6nccnc6cc5s4)ccc32)cc1]

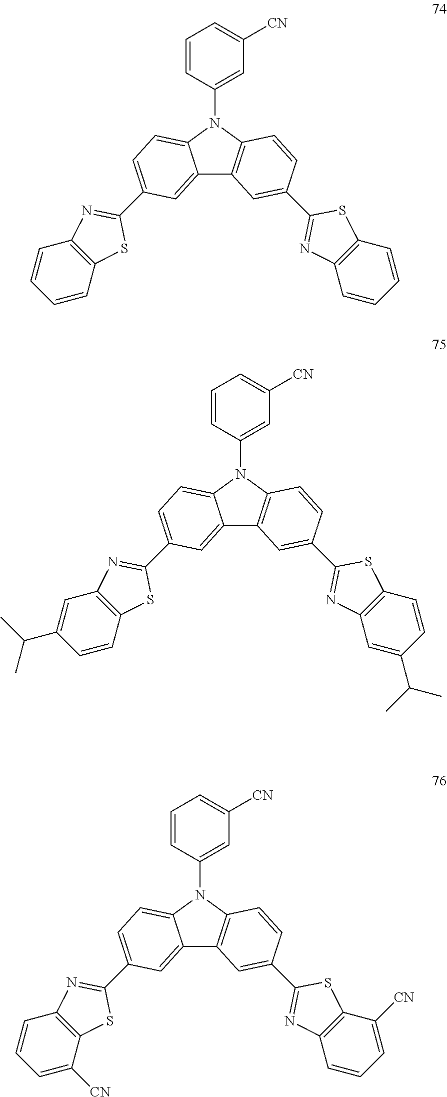 [SMILES: N#Cc1cccc(-n2c3ccc(-c4nc5ccccc5s4)cc3c3cc(-c4nc5ccccc5s4)ccc32)c1 ; CC(C)c1ccc2sc(-c3ccc4c(c3)c3cc(-c5nc6cc(C(C)C)ccc6s5)ccc3n4-c3cccc(C#N)c3)nc2c1 ; N#Cc1cccc(-n2c3ccc(-c4nc5cccc(C#N)c5s4)cc3c3cc(-c4nc5cccc(C#N)c5s4)ccc32)c1]

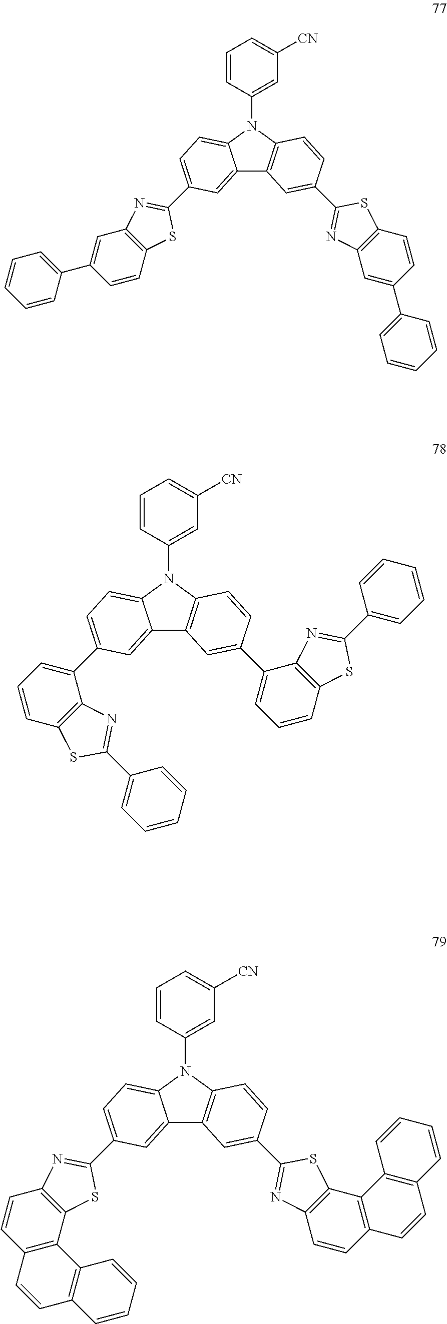 [SMILES: N#Cc1cccc(-n2c3ccc(-c4nc5cc(-c6ccccc6)ccc5s4)cc3c3cc(-c4nc5cc(-c6ccccc6)ccc5s4)ccc32)c1 ; N#Cc1cccc(-n2c3ccc(-c4cccc5sc(-c6ccccc6)nc45)cc3c3cc(-c4cccc5sc(-c6ccccc6)nc45)ccc32)c1 ; N#Cc1cccc(-n2c3ccc(-c4nc5ccc6ccc7ccccc7c6c5s4)cc3c3cc(-c4nc5ccc6ccc7ccccc7c6c5s4)ccc32)c1]

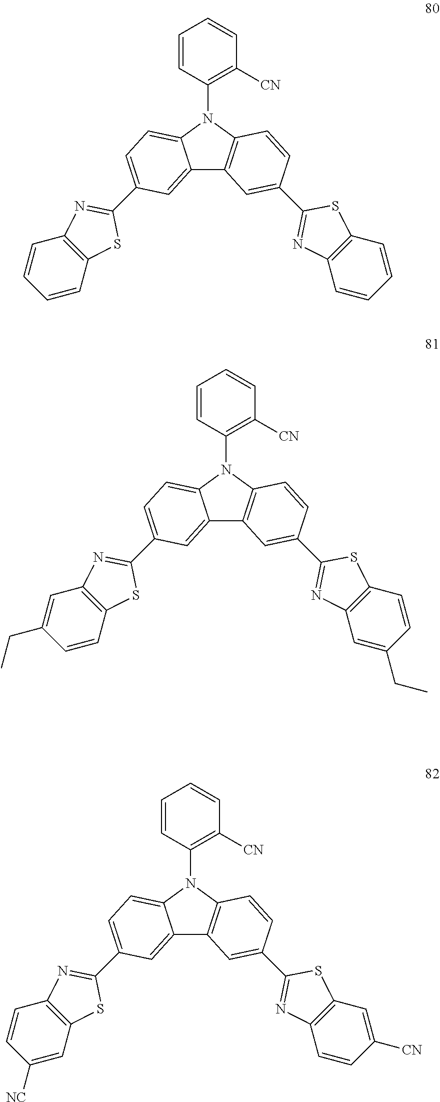 [SMILES: N#Cc1ccccc1-n1c2ccc(-c3nc4ccccc4s3)cc2c2cc(-c3nc4ccccc4s3)ccc21 ; CCc1ccc2sc(-c3ccc4c(c3)c3cc(-c5nc6cc(CC)ccc6s5)ccc3n4-c3ccccc3C#N)nc2c1 ; N#Cc1ccc2nc(-c3ccc4c(c3)c3cc(-c5nc6ccc(C#N)cc6s5)ccc3n4-c3ccccc3C#N)sc2c1]

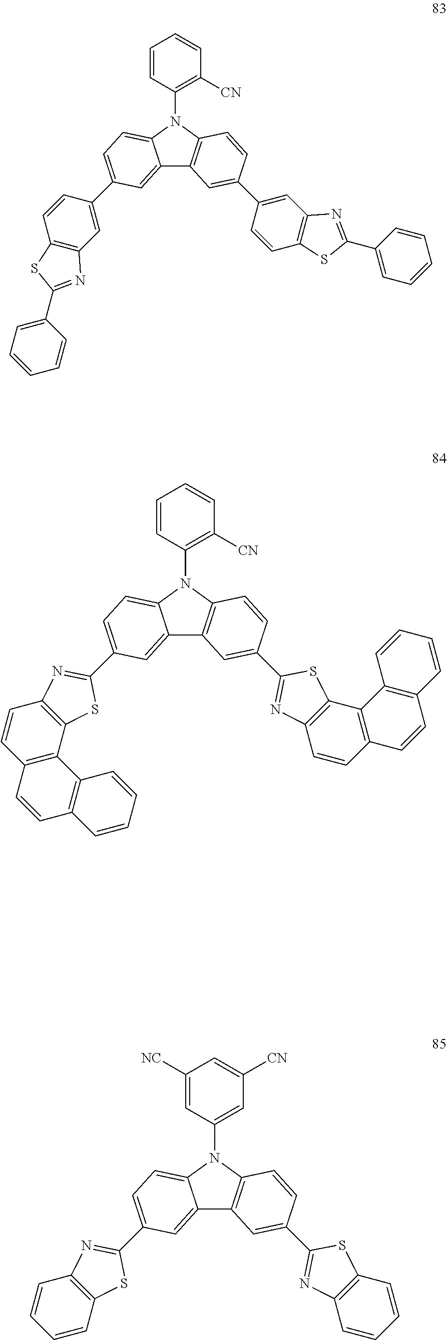 [SMILES: N#Cc1ccccc1-n1c2ccc(-c3ccc4sc(-c5ccccc5)nc4c3)cc2c2cc(-c3ccc4sc(-c5ccccc5)nc4c3)ccc21 ; N#Cc1ccccc1-n1c2ccc(-c3nc4ccc5ccc6ccccc6c5c4s3)cc2c2cc(-c3nc4ccc5ccc6ccccc6c5c4s3)ccc21 ; N#Cc1cc(C#N)cc(-n2c3ccc(-c4nc5ccccc5s4)cc3c3cc(-c4nc5ccccc5s4)ccc32)c1]

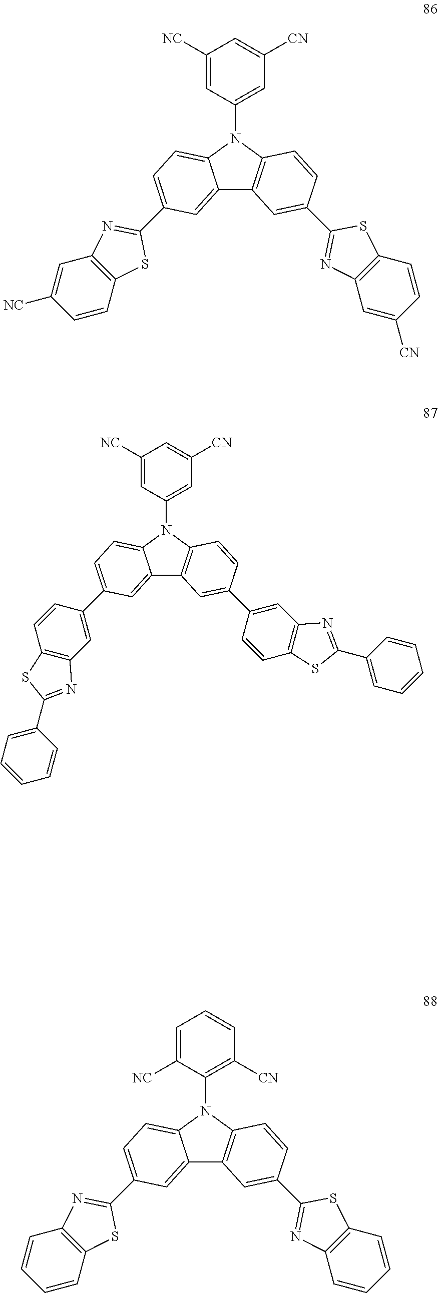 [SMILES: N#Cc1cc(C#N)cc(-n2c3ccc(-c4nc5cc(C#N)ccc5s4)cc3c3cc(-c4nc5cc(C#N)ccc5s4)ccc32)c1 ; N#Cc1cc(C#N)cc(-n2c3ccc(-c4ccc5sc(-c6ccccc6)nc5c4)cc3c3cc(-c4ccc5sc(-c6ccccc6)nc5c4)ccc32)c1 ; N#Cc1cccc(C#N)c1-n1c2ccc(-c3nc4ccccc4s3)cc2c2cc(-c3nc4ccccc4s3)ccc21]

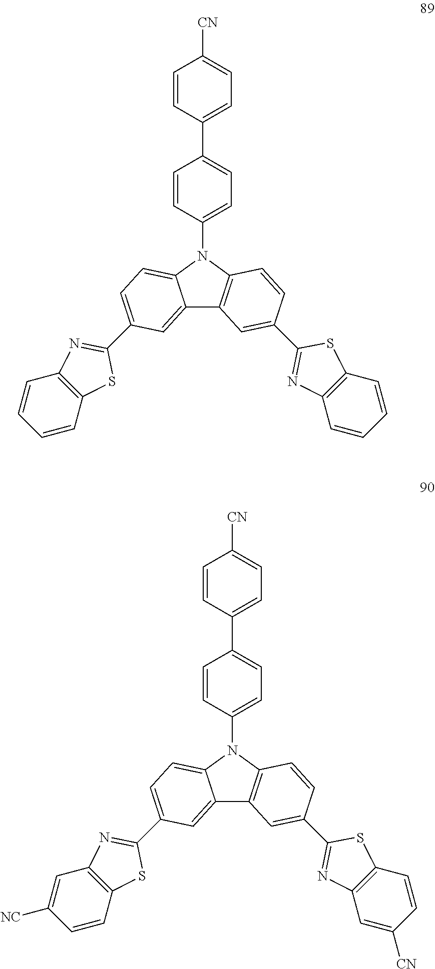 [SMILES: N#Cc1ccc(-c2ccc(-n3c4ccc(-c5nc6ccccc6s5)cc4c4cc(-c5nc6ccccc6s5)ccc43)cc2)cc1 ; N#Cc1ccc(-c2ccc(-n3c4ccc(-c5nc6cc(C#N)ccc6s5)cc4c4cc(-c5nc6cc(C#N)ccc6s5)ccc43)cc2)cc1]

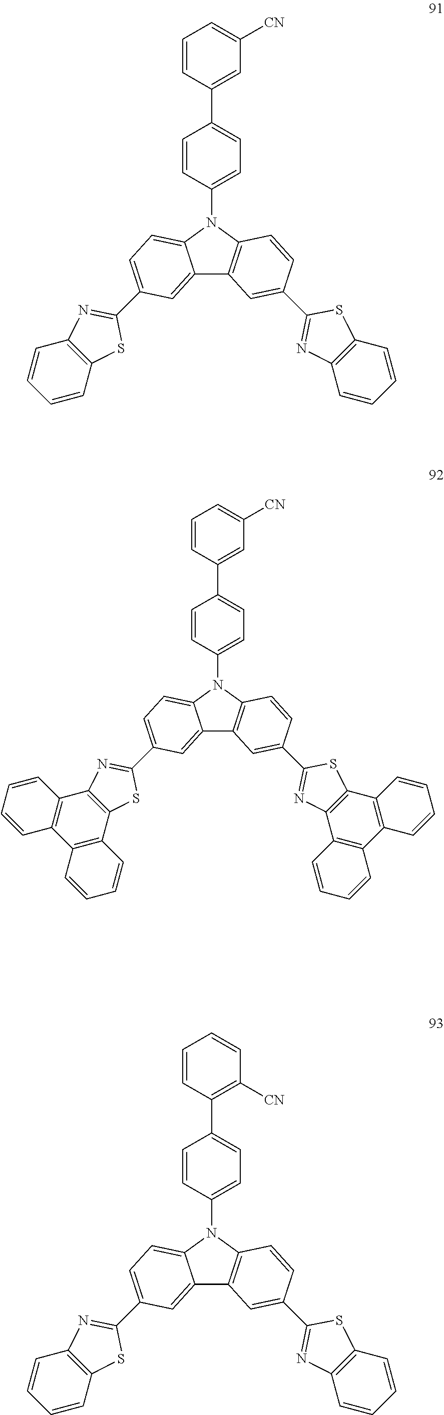 [SMILES: N#Cc1cccc(-c2ccc(-n3c4ccc(-c5nc6ccccc6s5)cc4c4cc(-c5nc6ccccc6s5)ccc43)cc2)c1 ; N#Cc1cccc(-c2ccc(-n3c4ccc(-c5nc6c7ccccc7c7ccccc7c6s5)cc4c4cc(-c5nc6c7ccccc7c7ccccc7c6s5)ccc43)cc2)c1 ; N#Cc1ccccc1-c1ccc(-n2c3ccc(-c4nc5ccccc5s4)cc3c3cc(-c4nc5ccccc5s4)ccc32)cc1]

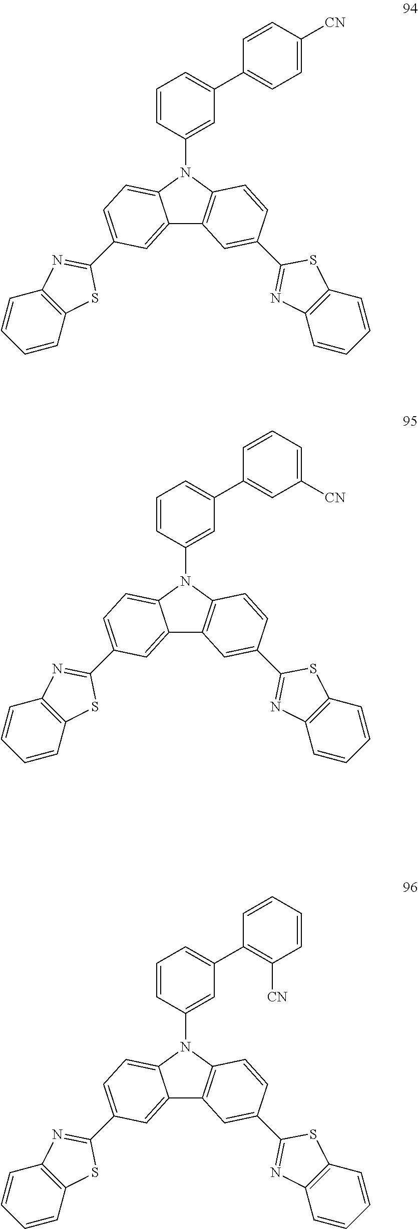 [SMILES: N#Cc1ccc(-c2cccc(-n3c4ccc(-c5nc6ccccc6s5)cc4c4cc(-c5nc6ccccc6s5)ccc43)c2)cc1 ; N#Cc1cccc(-c2cccc(-n3c4ccc(-c5nc6ccccc6s5)cc4c4cc(-c5nc6ccccc6s5)ccc43)c2)c1 ; N#Cc1ccccc1-c1cccc(-n2c3ccc(-c4nc5ccccc5s4)cc3c3cc(-c4nc5ccccc5s4)ccc32)c1]

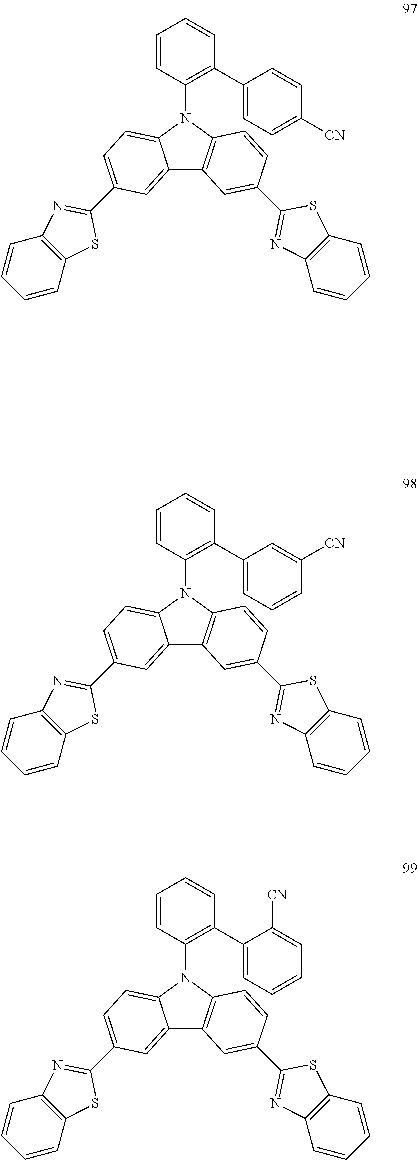 [SMILES: N#Cc1ccc(-c2ccccc2-n2c3ccc(-c4nc5ccccc5s4)cc3c3cc(-c4nc5ccccc5s4)ccc32)cc1 ; N#Cc1cccc(-c2ccccc2-n2c3ccc(-c4nc5ccccc5s4)cc3c3cc(-c4nc5ccccc5s4)ccc32)c1 ; N#Cc1ccccc1-c1ccccc1-n1c2ccc(-c3nc4ccccc4s3)cc2c2cc(-c3nc4ccccc4s3)ccc21]

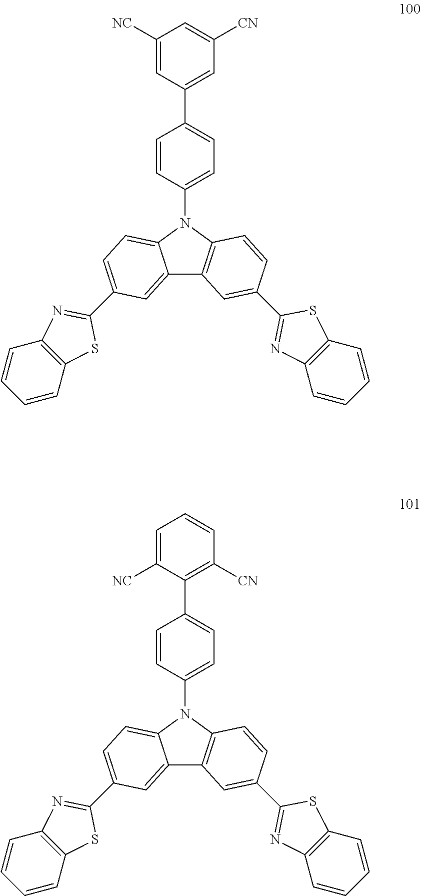 [SMILES: N#Cc1cc(C#N)cc(-c2ccc(-n3c4ccc(-c5nc6ccccc6s5)cc4c4cc(-c5nc6ccccc6s5)ccc43)cc2)c1 ; N#Cc1cccc(C#N)c1-c1ccc(-n2c3ccc(-c4nc5ccccc5s4)cc3c3cc(-c4nc5ccccc5s4)ccc32)cc1]

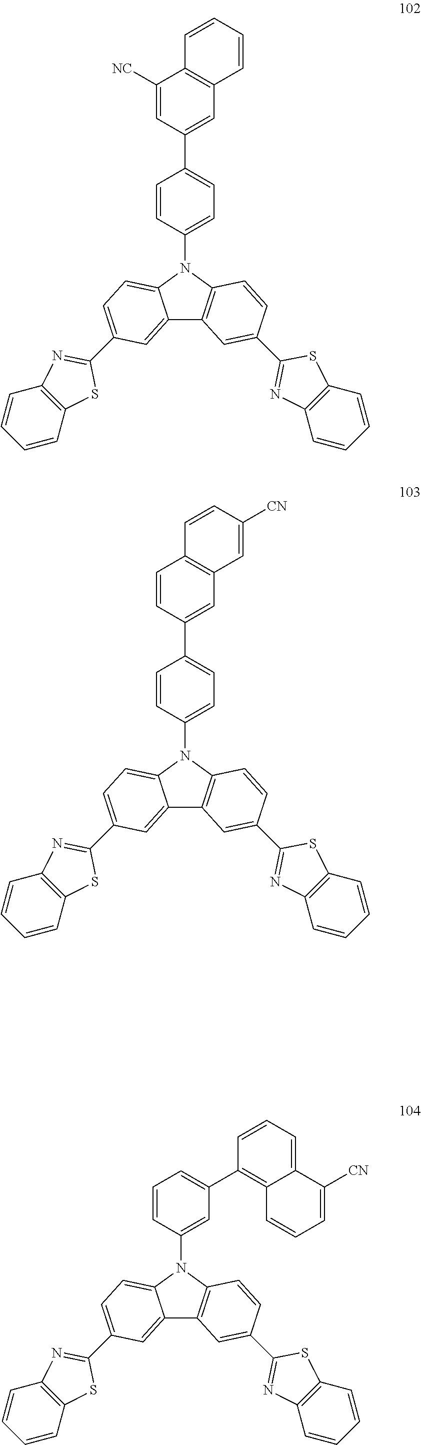 [SMILES: N#Cc1cc(-c2ccc(-n3c4ccc(-c5nc6ccccc6s5)cc4c4cc(-c5nc6ccccc6s5)ccc43)cc2)cc2ccccc12 ; N#Cc1ccc2ccc(-c3ccc(-n4c5ccc(-c6nc7ccccc7s6)cc5c5cc(-c6nc7ccccc7s6)ccc54)cc3)cc2c1 ; N#Cc1cccc2c(-c3cccc(-n4c5ccc(-c6nc7ccccc7s6)cc5c5cc(-c6nc7ccccc7s6)ccc54)c3)cccc12]

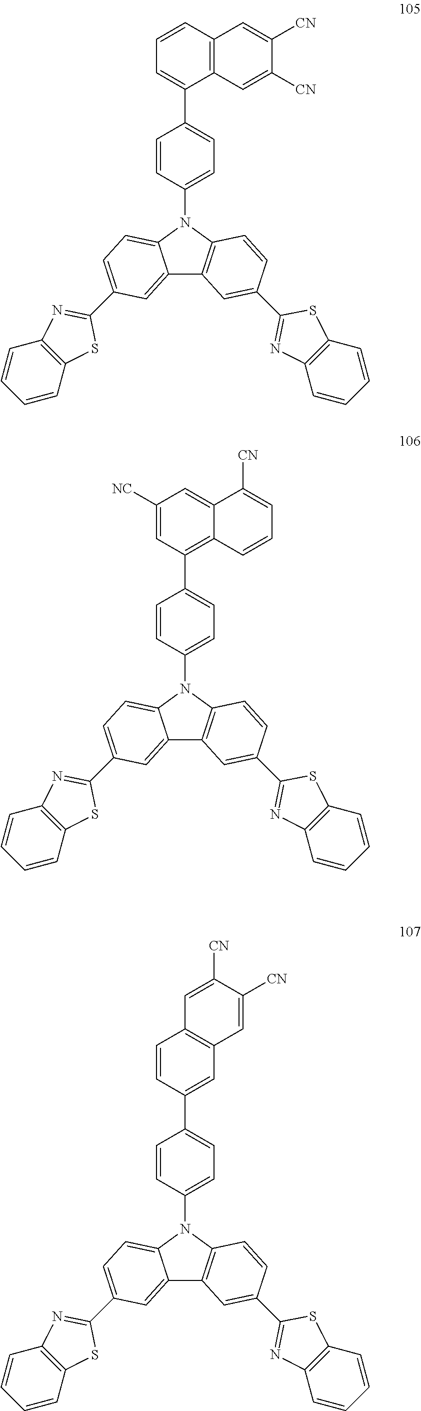 [SMILES: N#Cc1cc2cccc(-c3ccc(-n4c5ccc(-c6nc7ccccc7s6)cc5c5cc(-c6nc7ccccc7s6)ccc54)cc3)c2cc1C#N ; N#Cc1cc(-c2ccc(-n3c4ccc(-c5nc6ccccc6s5)cc4c4cc(-c5nc6ccccc6s5)ccc43)cc2)c2cccc(C#N)c2c1 ; N#Cc1cc2ccc(-c3ccc(-n4c5ccc(-c6nc7ccccc7s6)cc5c5cc(-c6nc7ccccc7s6)ccc54)cc3)cc2cc1C#N]

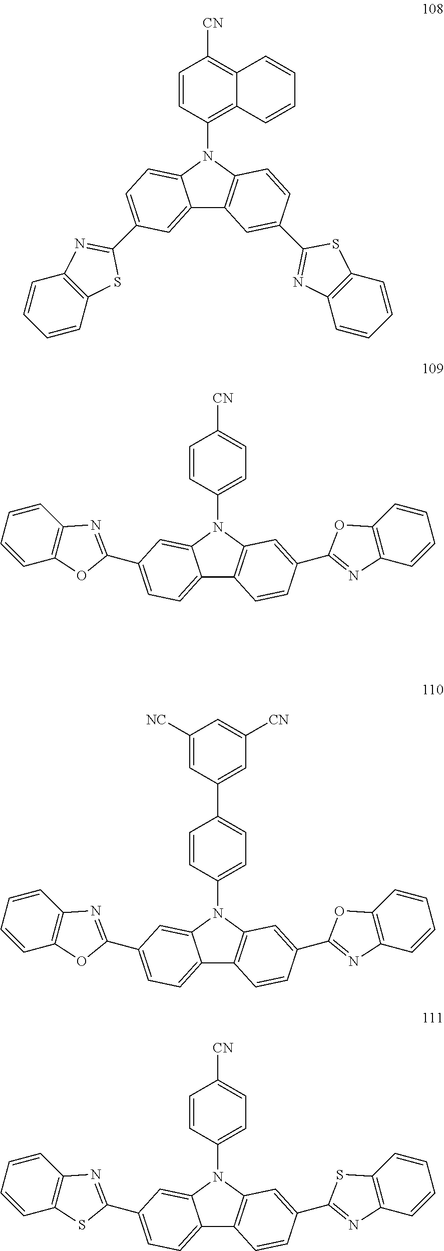 [SMILES: N#Cc1ccc(-n2c3ccc(-c4nc5ccccc5s4)cc3c3cc(-c4nc5ccccc5s4)ccc32)c2ccccc12 ; N#Cc1ccc(-n2c3cc(-c4nc5ccccc5o4)ccc3c3ccc(-c4nc5ccccc5o4)cc32)cc1 ; N#Cc1cc(C#N)cc(-c2ccc(-n3c4cc(-c5nc6ccccc6o5)ccc4c4ccc(-c5nc6ccccc6o5)cc43)cc2)c1 ; N#Cc1ccc(-n2c3cc(-c4nc5ccccc5s4)ccc3c3ccc(-c4nc5ccccc5s4)cc32)cc1]

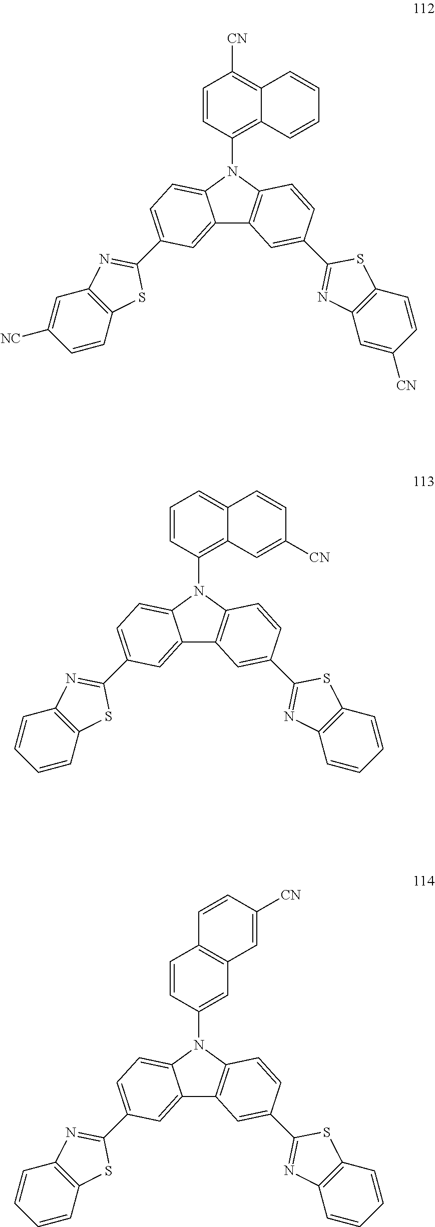 [SMILES: N#Cc1ccc2sc(-c3ccc4c(c3)c3cc(-c5nc6cc(C#N)ccc6s5)ccc3n4-c3ccc(C#N)c4ccccc34)nc2c1 ; N#Cc1ccc2cccc(-n3c4ccc(-c5nc6ccccc6s5)cc4c4cc(-c5nc6ccccc6s5)ccc43)c2c1 ; N#Cc1ccc2ccc(-n3c4ccc(-c5nc6ccccc6s5)cc4c4cc(-c5nc6ccccc6s5)ccc43)cc2c1]

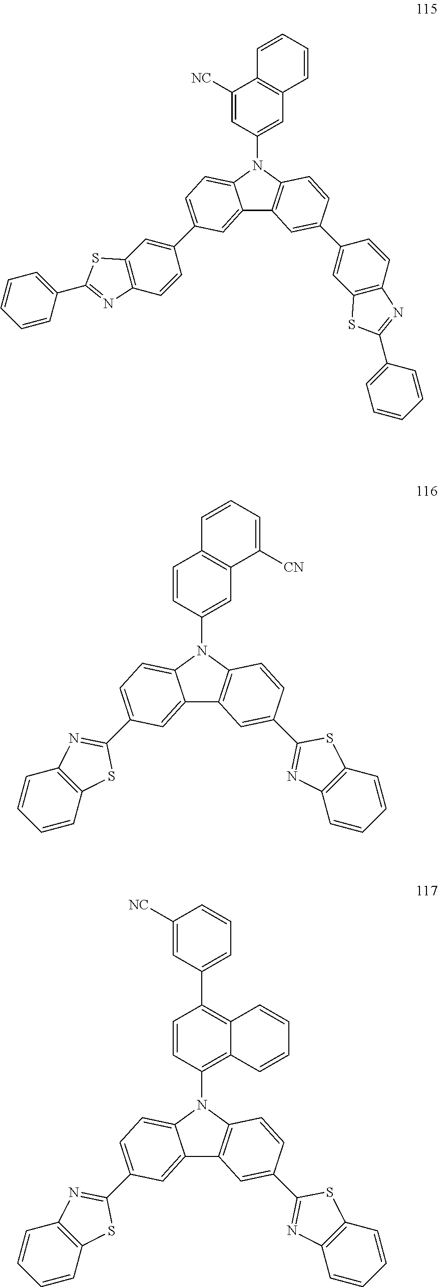 [SMILES: N#Cc1cc(-n2c3ccc(-c4ccc5nc(-c6ccccc6)sc5c4)cc3c3cc(-c4ccc5nc(-c6ccccc6)sc5c4)ccc32)cc2ccccc12 ; N#Cc1cccc2ccc(-n3c4ccc(-c5nc6ccccc6s5)cc4c4cc(-c5nc6ccccc6s5)ccc43)cc12 ; N#Cc1cccc(-c2ccc(-n3c4ccc(-c5nc6ccccc6s5)cc4c4cc(-c5nc6ccccc6s5)ccc43)c3ccccc23)c1]

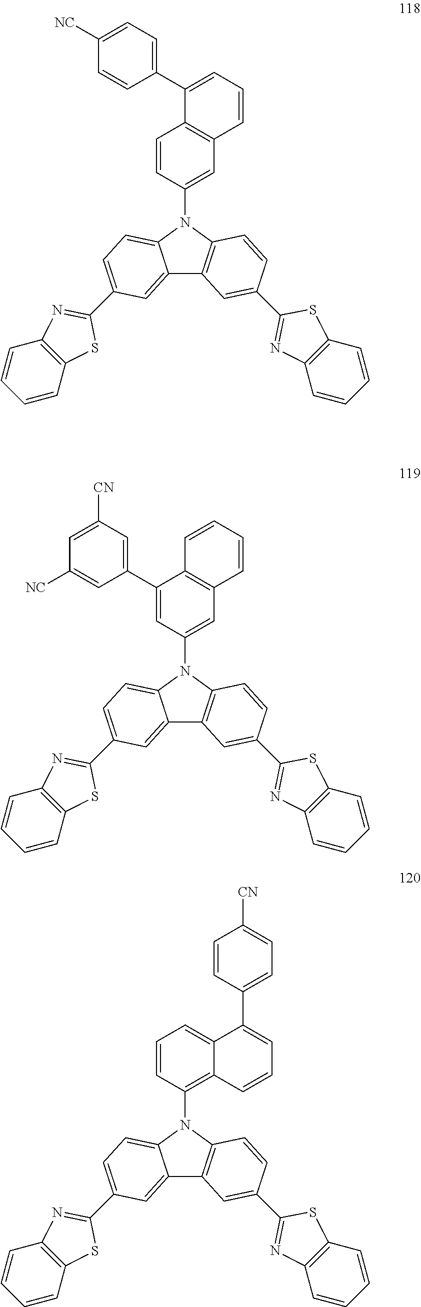 [SMILES: N#Cc1ccc(-c2cccc3cc(-n4c5ccc(-c6nc7ccccc7s6)cc5c5cc(-c6nc7ccccc7s6)ccc54)ccc23)cc1 ; N#Cc1cc(C#N)cc(-c2cc(-n3c4ccc(-c5nc6ccccc6s5)cc4c4cc(-c5nc6ccccc6s5)ccc43)cc3ccccc23)c1 ; N#Cc1ccc(-c2cccc3c(-n4c5ccc(-c6nc7ccccc7s6)cc5c5cc(-c6nc7ccccc7s6)ccc54)cccc23)cc1]

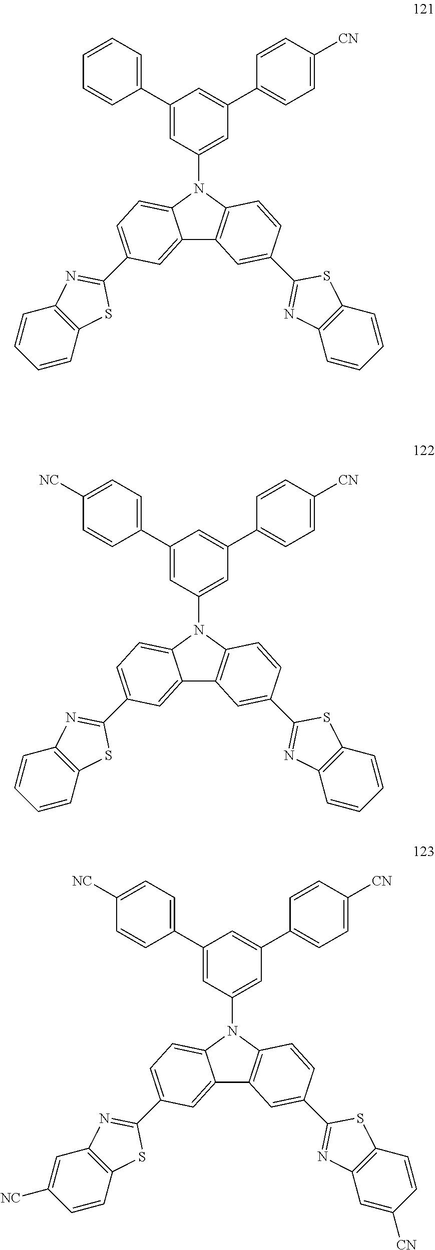 [SMILES: N#Cc1ccc(-c2cc(-c3ccccc3)cc(-n3c4ccc(-c5nc6ccccc6s5)cc4c4cc(-c5nc6ccccc6s5)ccc43)c2)cc1 ; N#Cc1ccc(-c2cc(-c3ccc(C#N)cc3)cc(-n3c4ccc(-c5nc6ccccc6s5)cc4c4cc(-c5nc6ccccc6s5)ccc43)c2)cc1 ; N#Cc1ccc(-c2cc(-c3ccc(C#N)cc3)cc(-n3c4ccc(-c5nc6cc(C#N)ccc6s5)cc4c4cc(-c5nc6cc(C#N)ccc6s5)ccc43)c2)cc1]

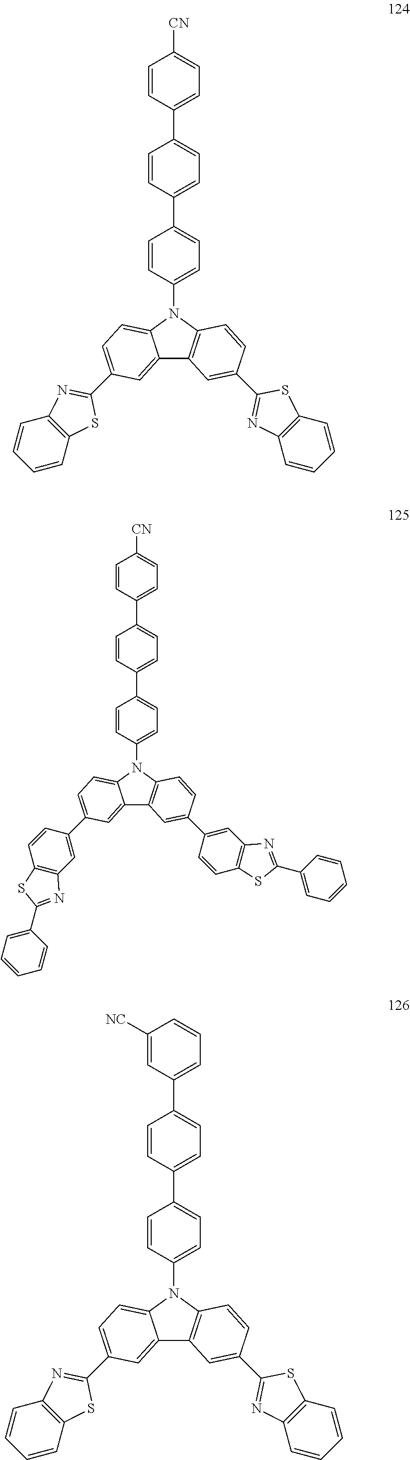 [SMILES: N#Cc1ccc(-c2ccc(-c3ccc(-n4c5ccc(-c6nc7ccccc7s6)cc5c5cc(-c6nc7ccccc7s6)ccc54)cc3)cc2)cc1 ; N#Cc1ccc(-c2ccc(-c3ccc(-n4c5ccc(-c6ccc7sc(-c8ccccc8)nc7c6)cc5c5cc(-c6ccc7sc(-c8ccccc8)nc7c6)ccc54)cc3)cc2)cc1 ; N#Cc1cccc(-c2ccc(-c3ccc(-n4c5ccc(-c6nc7ccccc7s6)cc5c5cc(-c6nc7ccccc7s6)ccc54)cc3)cc2)c1]

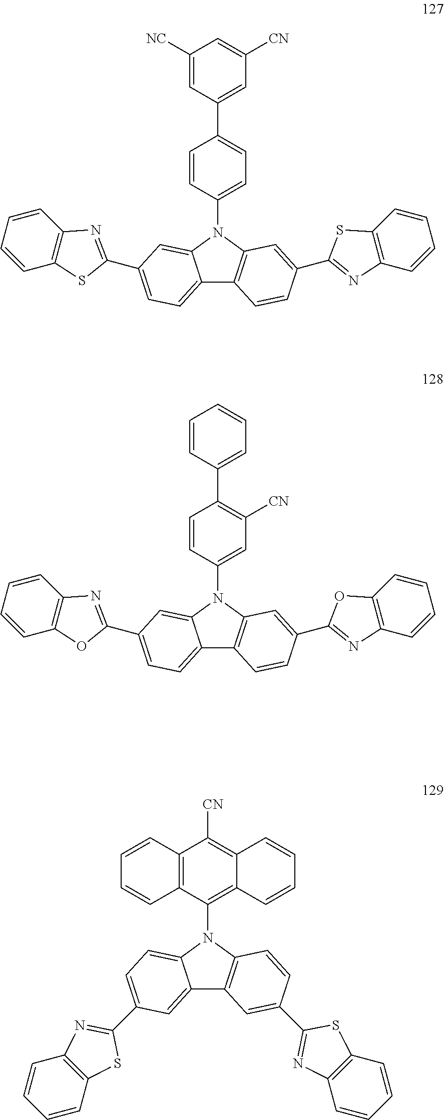 [SMILES: N#Cc1cc(C#N)cc(-c2ccc(-n3c4cc(-c5nc6ccccc6s5)ccc4c4ccc(-c5nc6ccccc6s5)cc43)cc2)c1 ; N#Cc1cc(-n2c3cc(-c4nc5ccccc5o4)ccc3c3ccc(-c4nc5ccccc5o4)cc32)ccc1-c1ccccc1 ; N#Cc1c2ccccc2c(-n2c3ccc(-c4nc5ccccc5s4)cc3c3cc(-c4nc5ccccc5s4)ccc32)c2ccccc12]

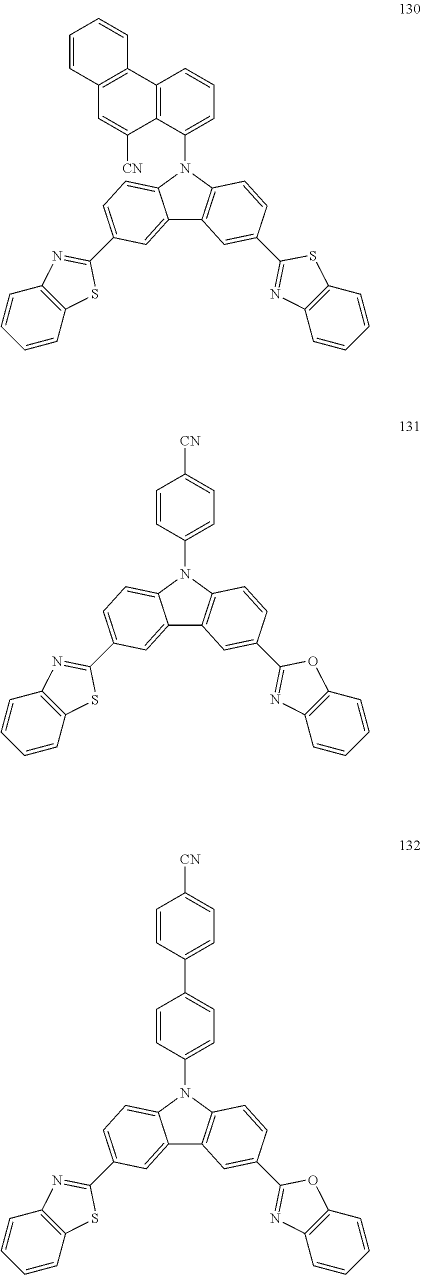 [SMILES: N#Cc1cc2ccccc2c2cccc(-n3c4ccc(-c5nc6ccccc6s5)cc4c4cc(-c5nc6ccccc6s5)ccc43)c12 ; N#Cc1ccc(-n2c3ccc(-c4nc5ccccc5o4)cc3c3cc(-c4nc5ccccc5s4)ccc32)cc1 ; N#Cc1ccc(-c2ccc(-n3c4ccc(-c5nc6ccccc6o5)cc4c4cc(-c5nc6ccccc6s5)ccc43)cc2)cc1]

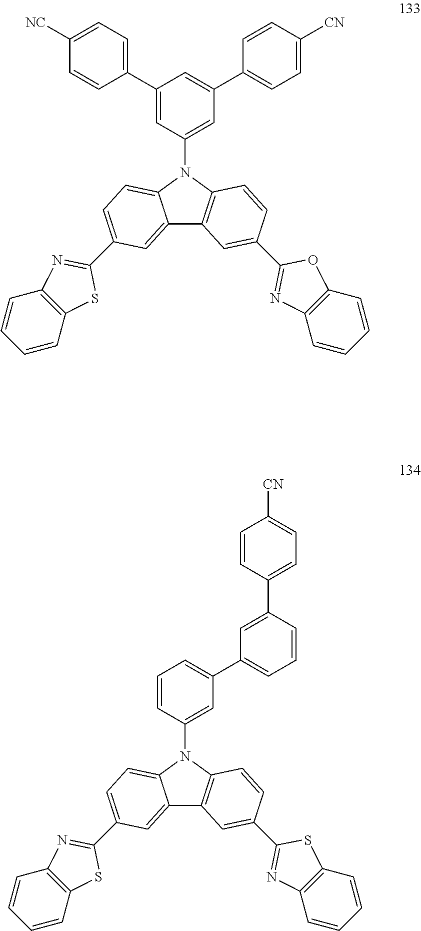 [SMILES: N#Cc1ccc(-c2cc(-c3ccc(C#N)cc3)cc(-n3c4ccc(-c5nc6ccccc6o5)cc4c4cc(-c5nc6ccccc6s5)ccc43)c2)cc1 ; N#Cc1ccc(-c2cccc(-c3cccc(-n4c5ccc(-c6nc7ccccc7s6)cc5c5cc(-c6nc7ccccc7s6)ccc54)c3)c2)cc1]

As described above, for the organic compound according to an embodiment of the present invention, a characteristic skeleton exhibiting unique characteristics and a moiety having unique characteristics introduced therein may be used to synthesize organic compounds having various characteristics, and as a result, the organic compound according to an embodiment of the present invention may be applied to a material for various organic layers such as a light emitting layer, a hole transport layer, an electron transport layer, an electron blocking layer, and a hole blocking layer, may preferably further improve luminescent properties such as luminous efficiency of the device as an electron transport material, and may also improve luminescent properties such as luminous efficiency even when employed in a light efficiency improving layer provided in an organic light emitting device.

In addition, the compound of an embodiment of the present invention may be applied to a device according to a general method for manufacturing an organic light emitting device.

An organic light emitting device according to an embodiment of the present invention may include a first electrode, a second electrode, and an organic layer arranged therebetween. The organic light emitting device may be manufactured using a general device manufacturing method and material, except that the organic compound of an embodiment of the present invention is used to form the organic layer of the device.

The organic layer of the organic light emitting device according to an embodiment of the present invention may have a monolayer structure or a multilayer structure in which two or more organic layers are stacked. For example, the structure of the organic layers may include a hole injecting layer, a hole transport layer, a light emitting layer, an electron transport layer, an electron injecting layer, an electron blocking layer, a hole blocking layer, and a light efficiency improving layer (capping layer). The number of the organic layers is not limited and may be increased or decreased.

In addition, the organic electroluminescent device may include a substrate, a first electrode (anode), an organic layer, a second electrode (cathode), and a light efficiency improving layer (capping layer), of which may be formed under the first electrode (bottom emission type) or on the second electrode (top emission type).

When the organic electroluminescent device is of a top emission type, light from the light emitting layer is emitted to the cathode and passes through the light efficiency improving layer (CPL) formed using the compound according to an embodiment of the present invention having a relatively high refractive index. The wavelength of the light is amplified, resulting in an increase in luminous efficiency. When the organic electroluminescent device is of a bottom emission type, the compound according to an embodiment of the present invention is employed in the light efficiency improving layer to improve the luminous efficiency of the organic electroluminescent device based on the same principle.

Preferred structures of the organic layers of the organic light emitting according to an embodiment of the present invention will be explained in more detail in the examples to be described later.

In addition, the organic electroluminescent device of an embodiment of the present invention may be manufactured by depositing a metal, a conductive metal oxide or an alloy thereof on a substrate by a physical vapor deposition (PVD) method such as sputtering or e-beam evaporation to form an anode, forming organic layers including a hole injecting layer, a hole transport layer, a light emitting layer, and an electron transport layer thereon, and depositing a cathode material thereon.

In addition to the above methods, the organic light emitting device may be fabricated by depositing a cathode material, organic layer materials, and an anode material in this order on a substrate. The organic layers may have a multilayer structure including a hole injecting layer, a hole transport layer, a light emitting layer, and an electron transport layer, but is not limited thereto and may have a monolayer structure. In addition, the organic layers may be manufactured in a smaller number of layers by a solvent process using various polymer materials rather than by a deposition process, such as spin coating, dip coating, doctor blading, screen printing, inkjet printing or thermal transfer.

As the anode material, a material having a high work function is generally preferred for easy injection of holes into the organic layers. Specific examples of anode materials suitable for use in an embodiment of the present invention include, but are not limited to: metals such as vanadium, chromium, copper, zinc, and gold and alloys thereof; metal oxides such as zinc oxide, indium oxide, indium thin oxide (ITO), and indium zinc oxide (IZO); combinations of metals and oxides such as ZnO:Al and SnO₂:Sb; and conductive polymers such as poly(3-methylthiophene), poly[3,4-(ethylene-1,2-dioxy)thiophene] (PEDT), polypyrrole, and polyaniline.

As the cathode material, a material having a low work function is generally preferred for easy injection of electrons into the organic layers. Specific examples of suitable cathode materials include, but are not limited to: metals such as magnesium, calcium, sodium, potassium, titanium, indium, yttrium, lithium, gadolinium, aluminum, silver, tin, and lead and alloys thereof; and multilayer structure materials such as LiF/Al and LiO₂/Al.

The hole injecting material is preferably a material that may receive holes injected from the anode at low voltage. The highest occupied molecular orbital (HOMO) of the hole injecting material is preferably between the work function of the anode material and the HOMO of the adjacent organic layer. Specific examples of hole injecting materials include, but are not limited to, metal porphyrin, oligothiophene, arylamine-based organic materials, hexanitrile hexaazatriphenylene, quinacridone-based organic materials, perylene-based organic materials, anthraquinone, polyaniline, and polythiophene-based conductive polymers.

The hole transport material is a material that may receive holes transported from the anode or the hole injecting layer and may transfer the holes to the light emitting layer. A material with high hole mobility is suitable. Specific examples thereof include arylamine-based organic materials, conductive polymers, and block copolymers consisting of conjugated and non-conjugated segments. The use of the organic compound according to an embodiment of the present invention ensures further improved low-voltage driving characteristics, high luminous efficiency, and life characteristics of the device.

The light emitting material is a material that may receive and recombine holes from the hole transport layer and electrons from the electron transport layer to emit light in the visible ray area. A material with high quantum efficiency for fluorescence and phosphorescence is preferred. Specific examples thereof include, but are not limited to, 8-hydroxyquinoline aluminum complex (Alq₃), carbazole-based compounds, dimerized styryl compounds, BAlq, 10-hydroxybenzoquinoline-metal compounds, benzoxazole-based compounds, benzthiazole-based compounds, and benzimidazole-based compounds, poly(p-phenylenevinylene) (PPV)-based polymers, spiro compounds, polyfluorene, and rubrene.

The electron transport material is a material that may receive electrons injected from the cathode and may transfer the electrons to the light emitting layer. A material with high electron mobility is suitable. Specific examples thereof include, but are not limited to, 8-hydroxyquinoline Al complex, Alq3 complexes, organic radical compounds, hydroxyflavone-metal complexes.

The organic light emitting device according to an embodiment of the present invention may be of a top emission, bottom emission or dual emission type according to the materials used.

In addition, the organic compound according to an embodiment of the present invention may perform its function even in organic electronic devices, including organic solar cells, organic photoconductors, and organic transistors, based on a similar principle to that applied to the organic light emitting device.

### MODE FOR CARRYING OUT INVENTION

Hereinafter, the present invention will be explained in more detail with reference to the preferred examples. However, these examples are provided for illustrative purposes and do not serve to limit the scope of the invention. It will be obvious to those skilled in the art that various modifications and changes are possible without departing from the scope and technical spirit of the present invention.

### Synthesis Example 1: Synthesis of Compound 1

### (1) Preparation Example 1: Synthesis of Intermediate 1-1

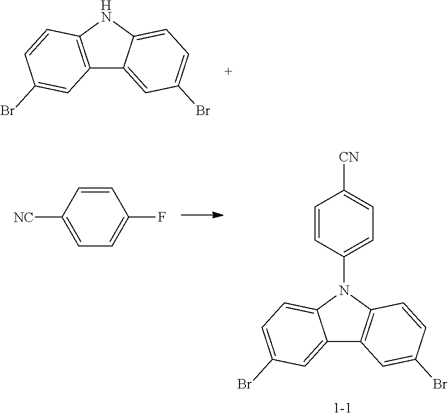 [SMILES: Brc1ccc2[nH]c3ccc(Br)cc3c2c1 ; N#Cc1ccc(F)cc1 ; N#Cc1ccc(-n2c3ccc(Br)cc3c3cc(Br)ccc32)cc1]

150 mL of DMF was added to 3,6-dibromocarbazole (10.0 g, 0.031 mol), 4-fluorobenzonitrile (4.47 g, 0.037 mol), and Cs₂CO₃ (6.38 g, 0.046 mol), and the resulting mixture was stirred under reflux at 150° C. for 12 hours and reacted. After completion of the reaction, the resulting product was extracted and concentrated, and then subjected to column chromatography to obtain 10.0 g (yield 76.3%) of <Intermediate 1-1>.

### (2) Preparation Example 2: Synthesis of Intermediate 1-2

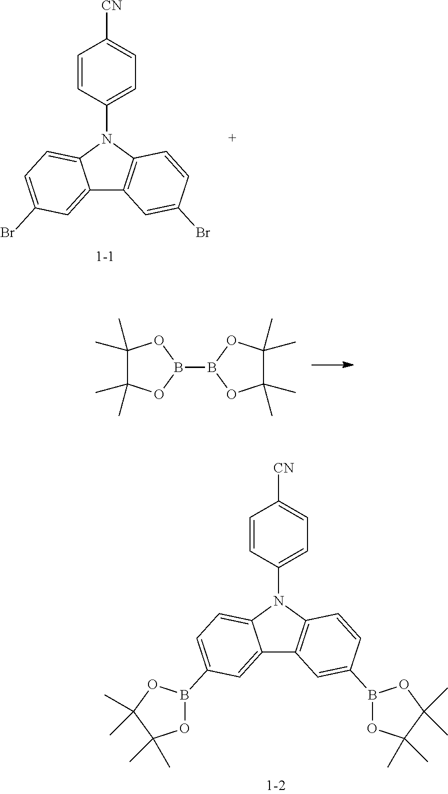

120 mL of dioxane was added to Intermediate 1-1 (10.0 g, 0.019 mol), bis(pinacolato)diboron (9.14 g, 0.046 mol), KOAc (15.94 g, 0.115 mol), and Pd(dppf)Cl₂ (10.44 g, 0.4 mmol), and the resulting mixture was stirred under reflux at 100° C. for 12 hours and reacted. After completion of the reaction, the resulting product was extracted and concentrated, and then subjected to column chromatography and recrystallization to obtain 9.1 g (yield 74.5%) of <Intermediate 1-2>.

### (3) Preparation Example 3: Synthesis of Compound 1

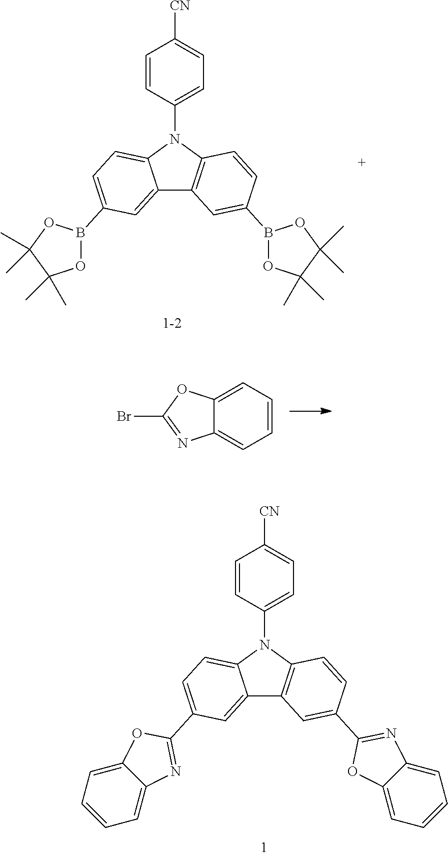

100 mL of toluene, 25 mL of EtOH, and 25 mL of H₂O were added to Intermediate 1-2 (10.0 g, 0.019 mol), 2-bromobenzoxazole (9.14 g, 0.046 mol), K₂CO₃ (15.94 g, 0.115 mol), and Pd(PPh₃)₄ (0.44 g, 0.4 mmol), and the resulting mixture was stirred under reflux at 100° C. for 6 hours and reacted. After completion of the reaction, the resulting product was extracted and concentrated, and then subjected to column chromatography and recrystallization to obtain 6.7 g (yield 69.4%) of <Compound 1>. LC/MS: m/z=502[(M+1)⁺]

### Synthesis Example 2: Synthesis of Compound 16

### (1) Preparation Example 1: Synthesis of Intermediate 16-1

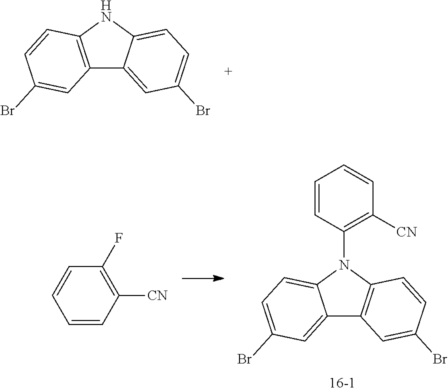 [SMILES: Brc1ccc2[nH]c3ccc(Br)cc3c2c1 ; N#Cc1ccccc1F ; N#Cc1ccccc1-n1c2ccc(Br)cc2c2cc(Br)ccc21]

150 mL of DMF was added to 3,6-dibromocarbazole (10.0 g, 0.031 mol), 2-fluorobenzonitrile (4.47 g, 0.037 mol), and Cs₂CO₃ (6.38 g, 0.046 mol), and the resulting mixture was stirred under reflux at 150° C. for 12 hours and reacted. After completion of the reaction, the resulting product was extracted and concentrated, and then subjected to column chromatography to obtain 9.9 g (yield 75.5%) of <Intermediate 16-1>.

### (2) Preparation Example 2: Synthesis of Intermediate 16-2

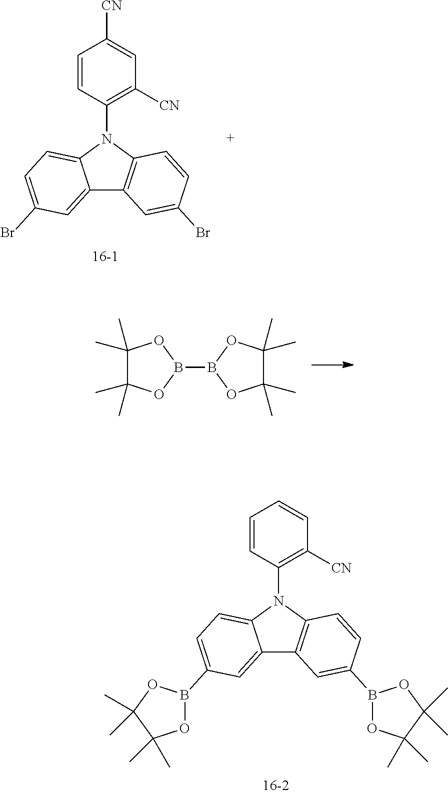

120 mL of dioxane was added to Intermediate 16-1 (10.0 g, 0.024 mol), bis(pinacolato)diboron (14.30 g, 0.056 mol), KOAc (9.21 g, 0.094 mol), and Pd(dppf)Cl₂ (1.03 g, 0.001 mol), and the resulting mixture was stirred under reflux at 100° C. for 12 hours and reacted. After completion of the reaction, the resulting product was extracted and concentrated, and then subjected to column chromatography and recrystallization to obtain 8.34 g (yield 68.3%) of <Intermediate 16-2>.

### (3) Preparation Example 3: Synthesis of Compound 16

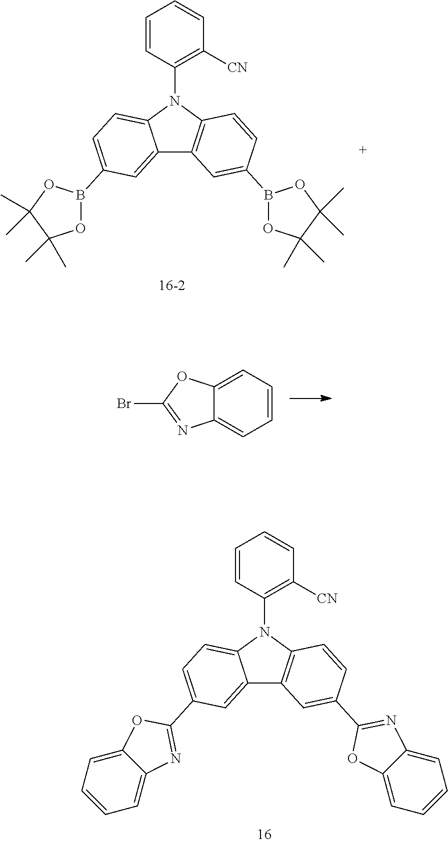

100 mL of toluene, 25 mL of EtOH, and 25 mL of H₂O were added to Intermediate 16-2 (10.0 g, 0.019 mol), 2-bromobenzoxazole (9.14 g, 0.046 mol), K₂CO₃ (15.94 g, 0.115 mol), and Pd(PPh₃)₄ (0.44 g, 0.4 mmol), and the resulting mixture was stirred under reflux at 100° C. for 6 hours and reacted. After completion of the reaction, the resulting product was extracted and concentrated, and then subjected to column chromatography and recrystallization to obtain 6.8 g (yield 70.4%) of <Compound 16>. LC/MS: m/z=502[(M+1)⁺]

### Synthesis Example 3: Synthesis of Compound 26

### (1) Preparation Example 1: Synthesis of Intermediate 26-1

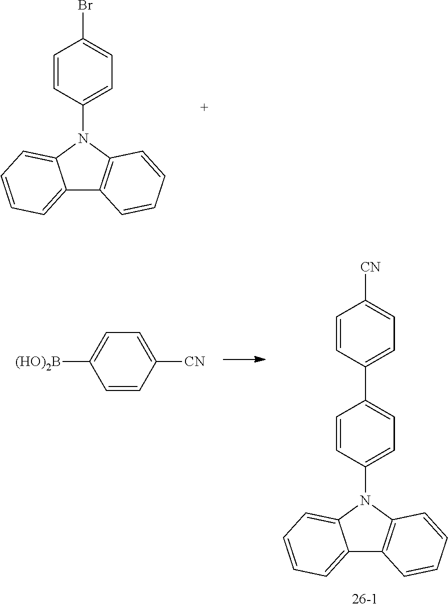 [SMILES: Brc1ccc(-n2c3ccccc3c3ccccc32)cc1 ; N#Cc1ccc(B(O)O)cc1 ; N#Cc1ccc(-c2ccc(-n3c4ccccc4c4ccccc43)cc2)cc1]

150 mL of toluene, 38 mL of EtOH, and 38 mL of H₂O were added to 9-(4-bromophenyl)-carbazole (10.0 g, 0.031 mol), 4-cyanophenylboronic acid (5.47 g, 0.037 mol), K₂CO₃ (12.87 g, 0.093 mol), and Pd(PPh₃)₄ (0.72 g, 0.6 mmol), and the resulting mixture was stirred under reflux at 100° C. for 6 hours and reacted. After completion of the reaction, the resulting product was extracted and concentrated, and then subjected to column chromatography to obtain 8.3 g (yield 77.7%) of <Intermediate 26-1>.

### (2) Preparation Example 2: Synthesis of Intermediate 26-2

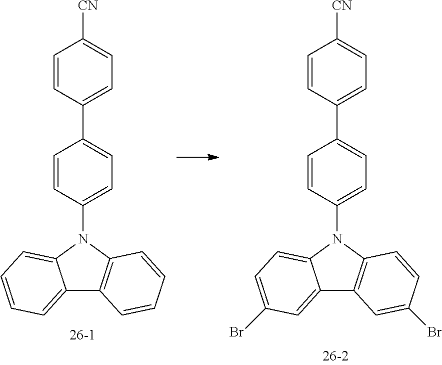 [SMILES: N#Cc1ccc(-c2ccc(-n3c4ccccc4c4ccccc43)cc2)cc1 ; N#Cc1ccc(-c2ccc(-n3c4ccc(Br)cc4c4cc(Br)ccc43)cc2)cc1]

150 mL of DMF was added to Intermediate 26-1 (10.0 g, 0.029 mol) and N-bromosuccinimide (12.4 g, 0.070 mol), and the resulting mixture was stirred under reflux at room temperature for 5 hours and reacted. After completion of the reaction, the resulting product was extracted and concentrated, and then subjected to column chromatography to obtain 9.86 g (yield 67.6%) of <Intermediate 26-2>.

### (3) Preparation Example 3: Synthesis of Intermediate 26-3

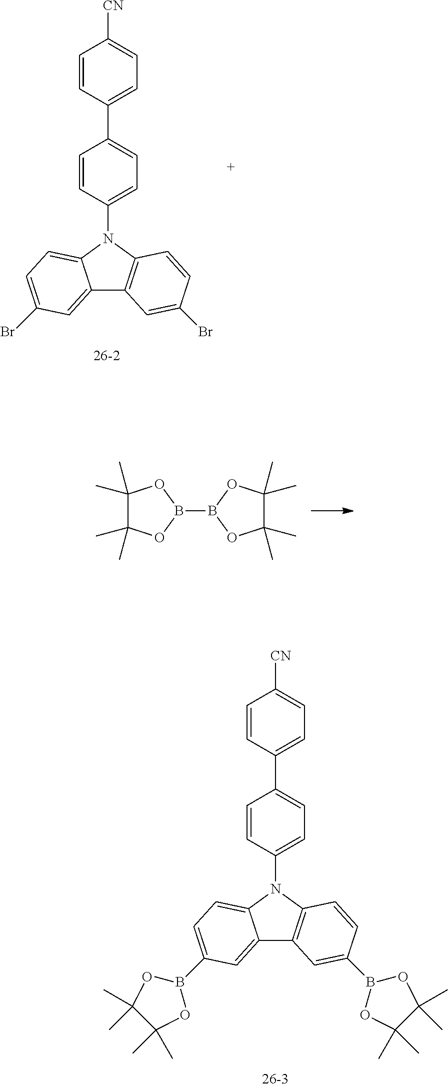

100 mL of dioxane was added to Intermediate 26-2 (10.0 g, 0.020 mol), bis(pinacolato)diboron (12.14 g, 0.048 mol), KOAc (7.82 g, 0.08 mol), and Pd(dppf)Cl₂ (0.87 g, 0.001 mol), and the resulting mixture was stirred at 100° C. for 12 hours and reacted. After completion of the reaction, the resulting product was extracted and concentrated, and then subjected to column chromatography and recrystallization to obtain 8.5 g (yield 71.6%) of <Intermediate 26-3>.

### (4) Preparation Example 4: Synthesis of Compound 26

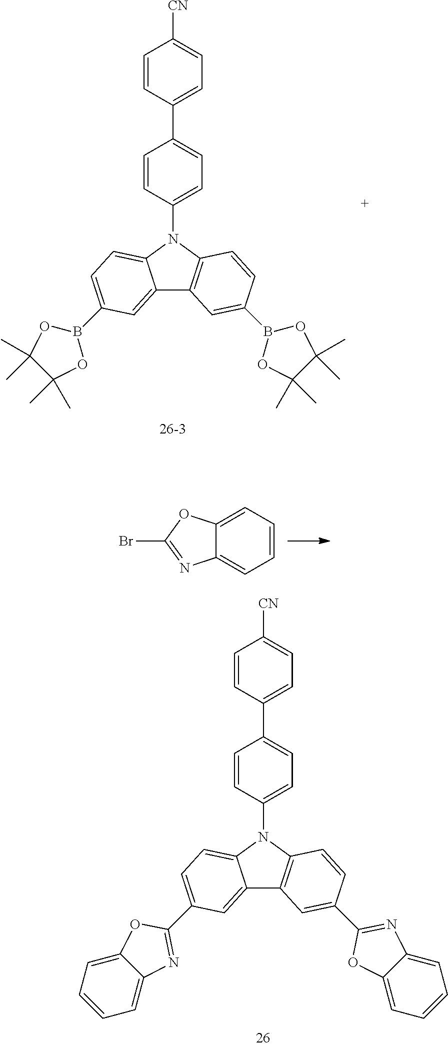

100 mL of toluene, 25 mL of ethanol, and 25 mL of H₂O were added to Intermediate 26-3 (10.0 g, 0.017 mol), 2-bromobenzoxazole (7.97 g, 0.040 mol), K₂CO₃ (13.91 g, 0.101 mol), and Pd(PPh₃)₄ (0.39 g, 0.3 mmol), and the resulting mixture was stirred under reflux at 100° C. for 6 hours and reacted. After completion of the reaction, the resulting product was extracted and concentrated, and then subjected to column chromatography and recrystallization to obtain 6.8 g (yield 70.1%) of <Compound 26>. LC/MS: m/z=578[(M+1)⁺]

### Synthesis Example 4: Synthesis of Compound 30

### (1) Preparation Example 1: Synthesis of Intermediate 30-1

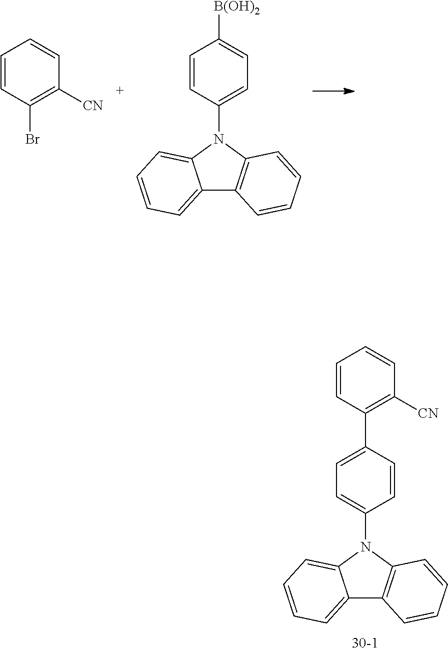 [SMILES: N#Cc1ccccc1Br ; OB(O)c1ccc(-n2c3ccccc3c3ccccc32)cc1 ; N#Cc1ccccc1-c1ccc(-n2c3ccccc3c3ccccc32)cc1]

280 mL of toluene, 70 mL of EtOH, and 70 mL of H₂O were added to 2-bromobenzonitrile (10.0 g, 0.055 mol), 4-(9-carbazolyl)phenylboronic acid (15.44 g, 0.066 mol), K₂CO₃ (22.78 g, 0.165 mol), and Pd(PPh₃)₄ (1.27 g, 0.001 mol), and the resulting mixture was stirred under reflux at 100° C. for 6 hours and reacted. After completion of the reaction, the resulting product was extracted and concentrated, and then subjected to column chromatography to obtain 15.7 g (yield 83.0%) of <Intermediate 30-1>.

### (2) Preparation Example 2: Synthesis of Intermediate 30-2

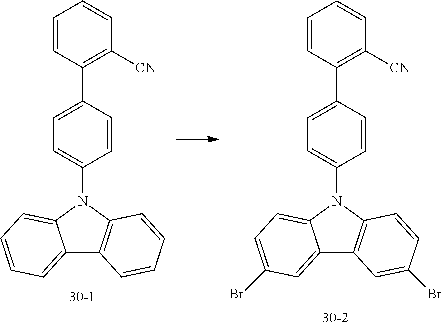 [SMILES: N#Cc1ccccc1-c1ccc(-n2c3ccccc3c3ccccc32)cc1 ; N#Cc1ccccc1-c1ccc(-n2c3ccc(Br)cc3c3cc(Br)ccc32)cc1]

150 mL of DMF was added to Intermediate 30-1 (10.0 g, 0.029 mol) and N-bromosuccinimide (12.4 g, 0.070 mol), and the resulting mixture was stirred under reflux at room temperature for 5 hours and reacted. After completion of the reaction, the resulting product was extracted and concentrated, and then subjected to column chromatography to obtain 10.0 g (yield 68.6%) of <Intermediate 30-2>.

### (3) Preparation Example 3: Synthesis of Intermediate 30-3

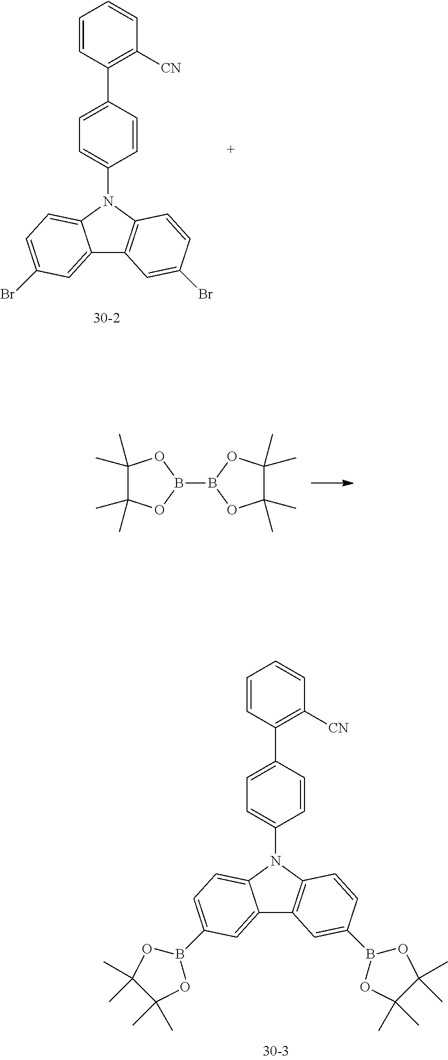

100 mL of dioxane was added to Intermediate 30-2 (10.0 g, 0.020 mol), bis(pinacolato)diboron (12.14 g, 0.048 mol), KOAc (7.82 g, 0.080 mol), and Pd(dppf)Cl₂ (0.87 g, 0.001 mol), and the resulting mixture was stirred under reflux at 100° C. for 12 hours and reacted. After completion of the reaction, the resulting product was extracted and concentrated, and then subjected to column chromatography and recrystallization to obtain 14.5 g (yield 75.4%) of <Intermediate 30-3>.

### (4) Preparation Example 4: Synthesis of Compound 30

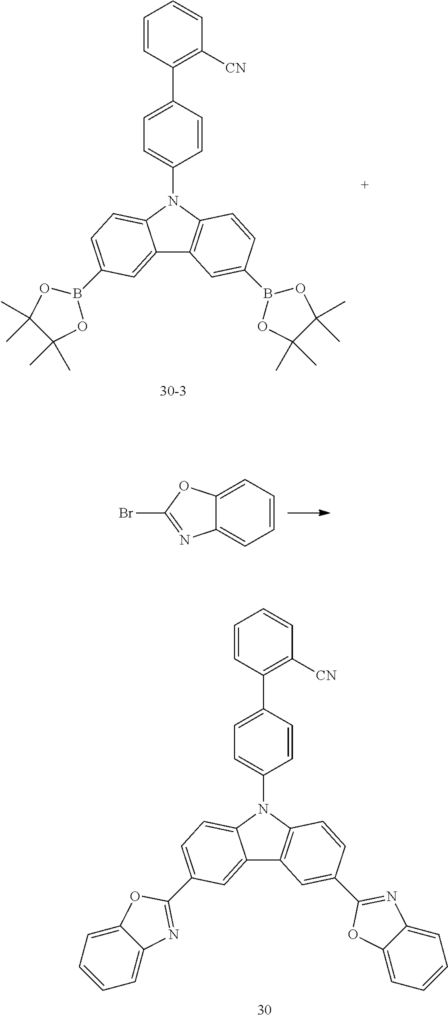

100 mL of toluene, 25 mL of EtOH, and 25 mL of H₂O were added to Intermediate 30-3 (10.0 g, 0.017 mol), 2-bromobenzoxazole (7.97 g, 0.040 mol), K₂CO₃ (13.91 g, 0.101 mol), and Pd(PPh₃)₄ (0.39 g, 0.3 mmol), and the resulting mixture was stirred under reflux at 100° C. for 6 hours and reacted. After completion of the reaction, the resulting product was extracted and concentrated, and then subjected to column chromatography and recrystallization to obtain 5.94 g (yield 70.5%) of <Compound 30>. LC/MS: m/z=578[(M+1)⁺]

### Synthesis Example 5: Synthesis of Compound 31

### (1) Preparation Example 1: Synthesis of Intermediate 31-1

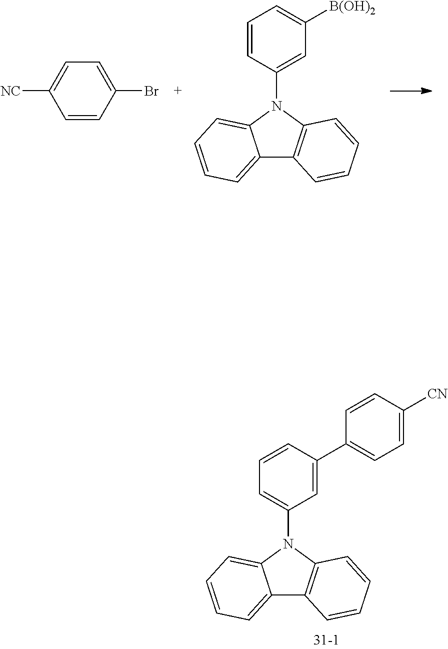 [SMILES: N#Cc1ccc(Br)cc1 ; OB(O)c1cccc(-n2c3ccccc3c3ccccc32)c1 ; N#Cc1ccc(-c2cccc(-n3c4ccccc4c4ccccc43)c2)cc1]

275 mL of toluene, 69 mL of EtOH, and 69 mL of H₂O were added to 4-bromobenzonitrile (10.0 g, 0.055 mol), 3-(9-carbazolyl)phenylboronic acid (17.35 g, 0.066 mol), K₂CO₃ (22.78 g, 0.165 mol), and Pd(PPh₃)₄ (1.27 g, 0.001 mol), and the resulting mixture was stirred under reflux at 100° C. for 6 hours and reacted. After completion of the reaction, the resulting product was extracted and concentrated, and then subjected to column chromatography to obtain 15.0 g (yield 79.3%) of <Intermediate 31-1>.

### (2) Preparation Example 2: Synthesis of Intermediate 31-2

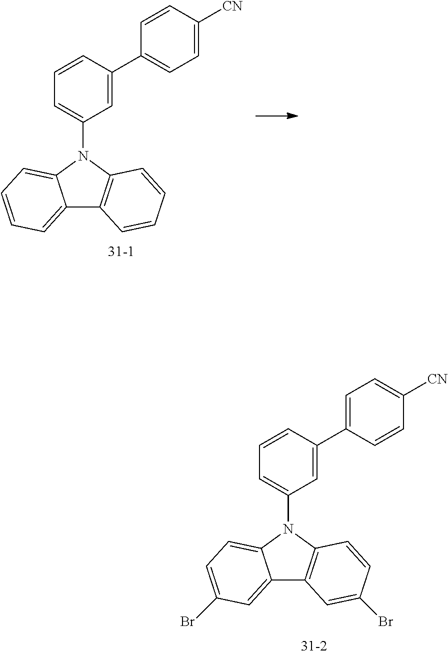 [SMILES: N#Cc1ccc(-c2cccc(-n3c4ccccc4c4ccccc43)c2)cc1 ; N#Cc1ccc(-c2cccc(-n3c4ccc(Br)cc4c4cc(Br)ccc43)c2)cc1]

150 mL of DMF was added to Intermediate 31-1 (10.0 g, 0.029 mol) and N-bromosuccinimide (18.6 g, 0.070 mol), and the resulting mixture was stirred under reflux at room temperature for 5 hours and reacted. After completion of the reaction, the resulting product was extracted and concentrated, and then subjected to column chromatography to obtain 9.7 g (yield 66.5%) of <Intermediate 31-2>.

### (3) Preparation Example 3: Synthesis of Intermediate 31-3

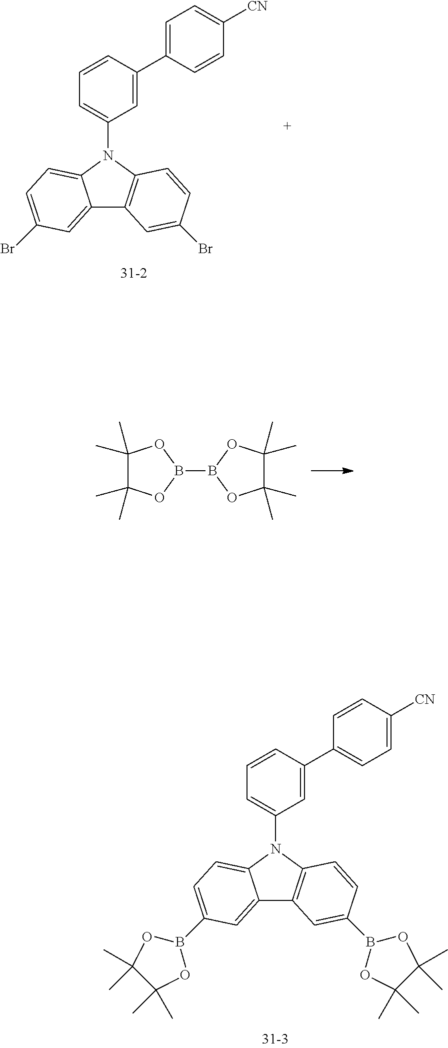

100 mL of dioxane was added to Intermediate 31-2 (10.0 g, 0.020 mol), bis(pinacolato)diboron (12.14 g, 0.048 mol), KOAc (7.82 g, 0.080 mol), and Pd(dppf)Cl₂ (0.87 g, 0.001 mol), and the resulting mixture was stirred under reflux at 100° C. for 12 hours and reacted. After completion of the reaction, the resulting product was extracted and concentrated, and then subjected to column chromatography and recrystallization to obtain 8.4 g (yield 70.7%) of <Intermediate 31-3>.

### (4) Preparation Example 4: Synthesis of Intermediate 31

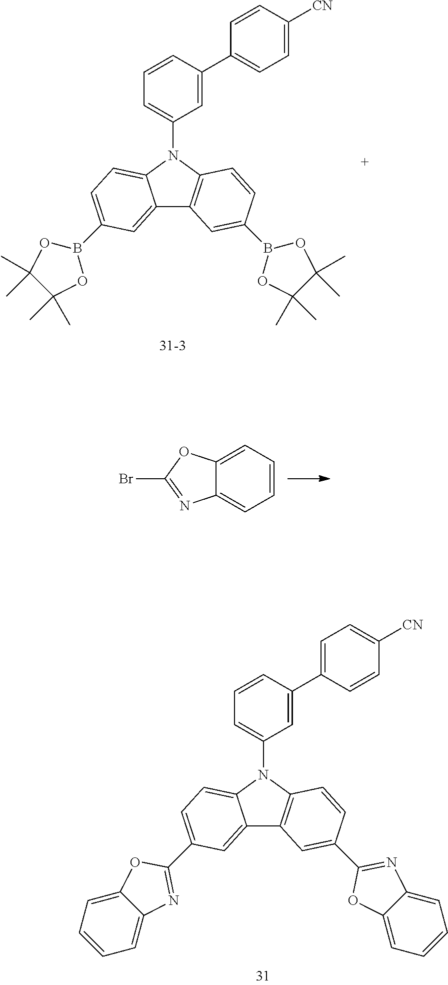

100 mL of toluene, 25 mL of EtOH, and 25 mL of H₂O were added to Intermediate 31-3 (10.0 g, 0.017 mol), 2-bromobenzoxazole (7.97 g, 0.040 mol), K₂CO₃ (13.91 g, 0.101 mol), and Pd(PPh₃)₄ (0.39 g, 0.3 mmol), and the resulting mixture was stirred under reflux at 100° C. for 6 hours and reacted. After completion of the reaction, the resulting product was extracted and concentrated, and then subjected to column chromatography and recrystallization to obtain 6.8 g (yield 70.0%) of <Compound 31>. LC/MS: m/z=578[(M+1)⁺]

### Synthesis Example 6: Synthesis of Compound 35

### (1) Preparation Example 1: Synthesis of Intermediate 35-1

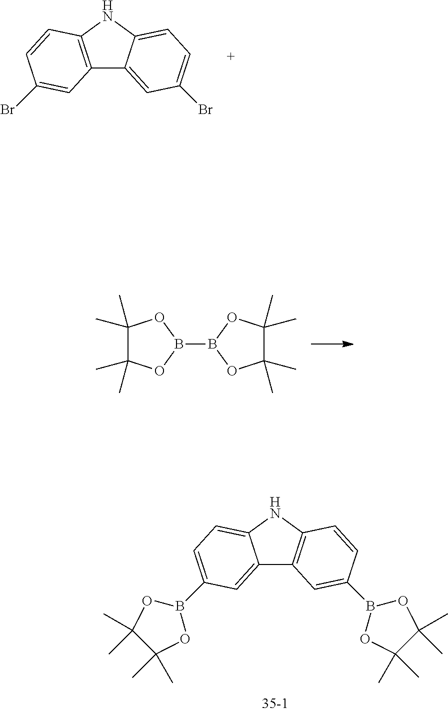

155 mL of dioxane was added to 3,6-dibromocarbazole (10.0 g, 0.031 mol), bis(pinacolato)diboron (18.75 g, 0.074 mol), KOAc (12.08 g, 0.123 mol), and Pd(dppf)Cl₂ (1.35 g, 0.002 mol), and the resulting mixture was stirred at 100° C. for 12 hours and reacted. After completion of the reaction, the resulting product was extracted and concentrated, and then subjected to column chromatography to obtain 9.9 g (yield 80.2%) of <Intermediate 35-1>.

### (2) Preparation Example 2: Synthesis of Intermediate 35-2

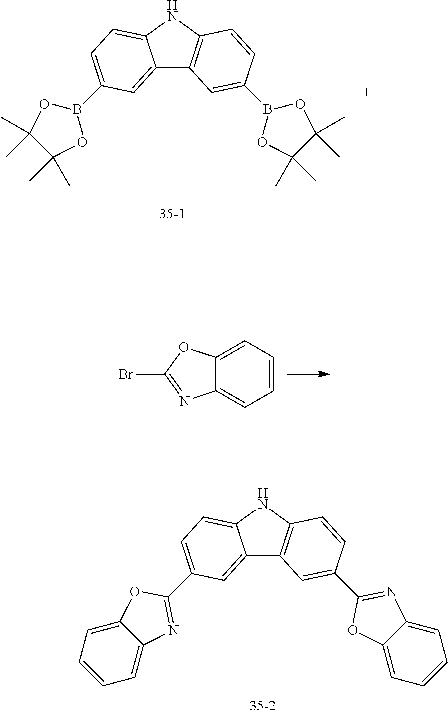

120 mL of toluene, 30 mL of EtOH, and 30 mL of H₂O were added to Intermediate 35-1 (10.0 g, 0.024 mol), 2-bromobenzoxazole (11.34 g, 0.057 mol), K₂CO₃ (19.79 g, 0.143 mol), and Pd(PPh₃)₄ (0.55 g, 0.5 mmol), and the resulting mixture was stirred under reflux at 100° C. for 6 hours and reacted. After completion of the reaction, the resulting product was extracted and concentrated, and then subjected to column chromatography to obtain 7.2 g (yield 75.2%) of <Intermediate 35-2>.

### (3) Preparation Example 3: Synthesis of Intermediate 35-3

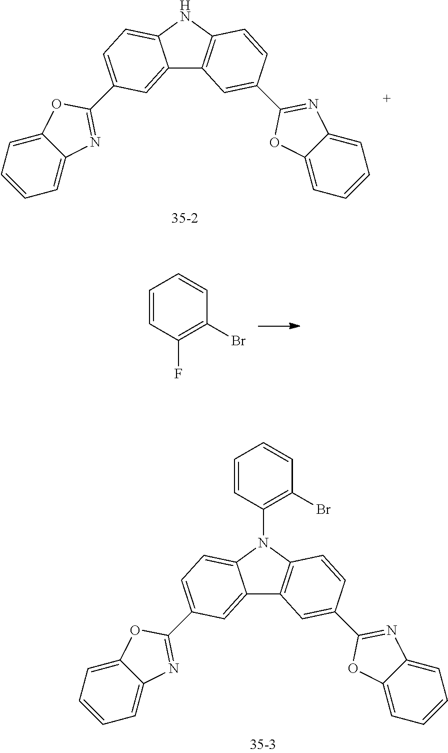 [SMILES: c1ccc2oc(-c3ccc4[nH]c5ccc(-c6nc7ccccc7o6)cc5c4c3)nc2c1 ; Fc1ccccc1Br ; Brc1ccccc1-n1c2ccc(-c3nc4ccccc4o3)cc2c2cc(-c3nc4ccccc4o3)ccc21]

130 mL of DMF was added to Intermediate 35-2 (10.0 g, 0.025 mol), 1-bromo-2-fluorobenzene (7.59 g, 0.030 mol), and Cs₂CO₃ (5.16 g, 0.037 mol), and the resulting mixture was stirred under reflux at 150° C. for 12 hours and reacted. After completion of the reaction, the resulting product was extracted and concentrated, and then subjected to column chromatography and recrystallization to obtain 9.8 g (yield 70.7%) of <Intermediate 35-3>.

### (4) Preparation Example 4: Synthesis of Compound 35

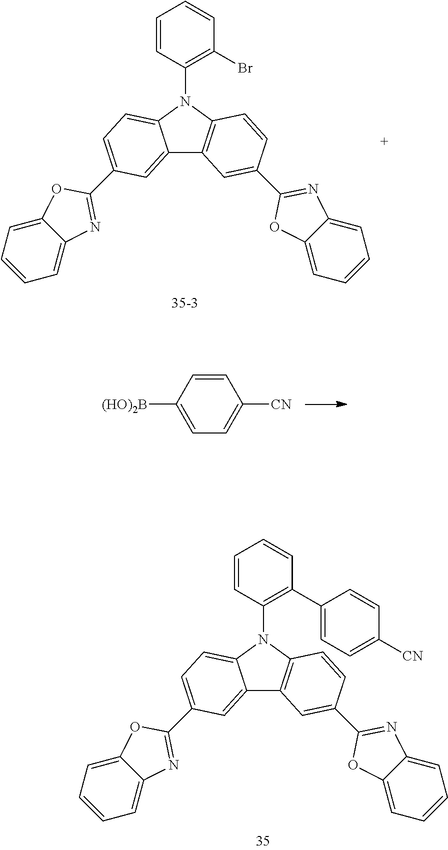 [SMILES: Brc1ccccc1-n1c2ccc(-c3nc4ccccc4o3)cc2c2cc(-c3nc4ccccc4o3)ccc21 ; N#Cc1ccc(B(O)O)cc1 ; N#Cc1ccc(-c2ccccc2-n2c3ccc(-c4nc5ccccc5o4)cc3c3cc(-c4nc5ccccc5o4)ccc32)cc1]

120 mL of toluene, 30 mL of EtOH, and 30 mL of H₂O were added to Intermediate 35-3 (10.0 g, 0.018 mol), 4-cyanophenylboronic acid (3.17 g, 0.022 mol), K₂CO₃ (7.45 g, 0.054 mol), and Pd(PPh₃)₄ (0.42 g, 0.4 mmol), and the resulting mixture was stirred under reflux at 100° C. for 6 hours and reacted. After completion of the reaction, the resulting product was extracted and concentrated, and then subjected to column chromatography and recrystallization to obtain 6.8 g (yield 65.4%) of <Compound 35>.

LC/MS: m/z=578[(M+1)⁺]

### Synthesis Example 7: Synthesis of Compound 38

### (1) Preparation Example 1: Synthesis of Intermediate 38-1

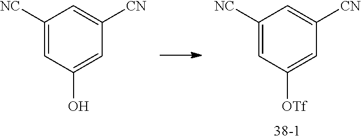 [SMILES: N#Cc1cc(O)cc(C#N)c1 ; N#Cc1cc(C#N)cc(OS(=O)(=O)C(F)(F)F)c1]

Tf₂O (19.7 g, 0.153 mol) was added dropwise to 5-hydroxyisophthalonitrile (10.0 g, 0.069 mol) and Et₃N in dichloromethane (9.78 g, 0.153 mol). The resulting mixture was slowly warmed to room temperature, and then stirred under reflux for 12 hours and reacted. After completion of the reaction, the resulting product was extracted and subjected to column purification to obtain 16.7 g (yield 87.2%) of <Intermediate 38-1>.

### (2) Preparation Example 2: Synthesis of Intermediate 38-2

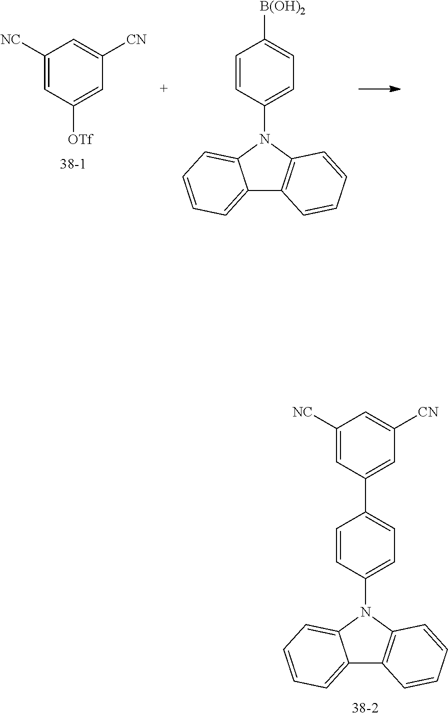 [SMILES: N#Cc1cc(C#N)cc(OS(=O)(=O)C(F)(F)F)c1 ; OB(O)c1ccc(-n2c3ccccc3c3ccccc32)cc1 ; N#Cc1cc(C#N)cc(-c2ccc(-n3c4ccccc4c4ccccc43)cc2)c1]

180 mL of dioxane and 18 mL of H₂O were added to Intermediate 38-1 (10.0 g, 0.036 mol), 4-(9-carbazolyl)phenylboronic acid (12.47 g, 0.043 mol), K₂CO₃ (15.01 g, 0.109 mol), and Pd(PPh₃)₄ (0.84 g, 0.7 mmol), and the resulting mixture was stirred under reflux at 100° C. for 4 hours and reacted. After completion of the reaction, the resulting product was extracted and concentrated, and then subjected to column chromatography to obtain 9.9 g (yield 74.0%) of <Intermediate 38-2>.

### (3) Preparation Example 3: Synthesis of Intermediate 38-3

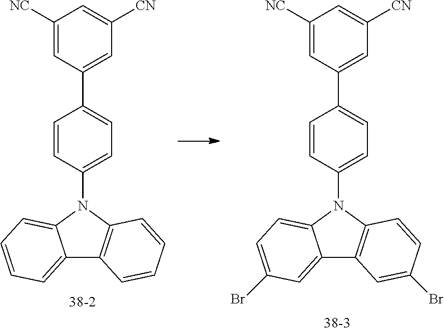 [SMILES: N#Cc1cc(C#N)cc(-c2ccc(-n3c4ccccc4c4ccccc43)cc2)c1 ; N#Cc1cc(C#N)cc(-c2ccc(-n3c4ccc(Br)cc4c4cc(Br)ccc43)cc2)c1]

140 mL of DMF was added to Intermediate 38-2 (10.0 g, 0.027 mol) and N-bromosuccinimide (11.6 g, 0.065 mol), and the resulting mixture was stirred under reflux at room temperature for 5 hours and reacted. After completion of the reaction, the resulting product was extracted and concentrated, and then subjected to column chromatography to obtain 9.1 g (yield 63.8%) of <Intermediate 38-3>.

### (4) Preparation Example 4: Synthesis of Intermediate 38-4

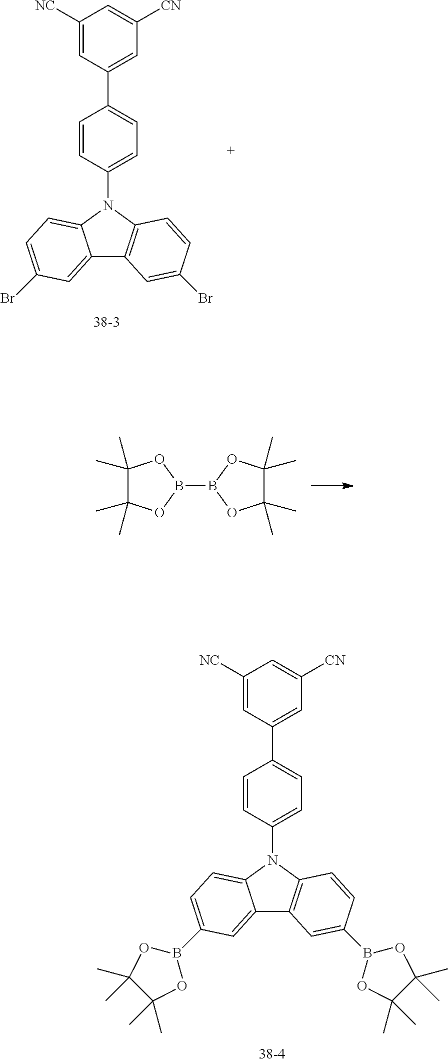

100 mL of dioxane was added to Intermediate 38-3 (10.0 g, 0.019 mol), bis(pinacolato)diboron (11.56 g, 0.046 mol), KOAc (7.45 g, 0.076 mol), and Pd(dppf)Cl₂ (0.83 g, 0.001 mol), and the resulting mixture was stirred under reflux at 100° C. for 12 hours and reacted. After completion of the reaction, the resulting product was extracted and concentrated, and then subjected to column chromatography and recrystallization to obtain 7.9 g (yield 67.0%) of <Intermediate 38-4>.

### (5) Preparation Example 5: Synthesis of Compound 38

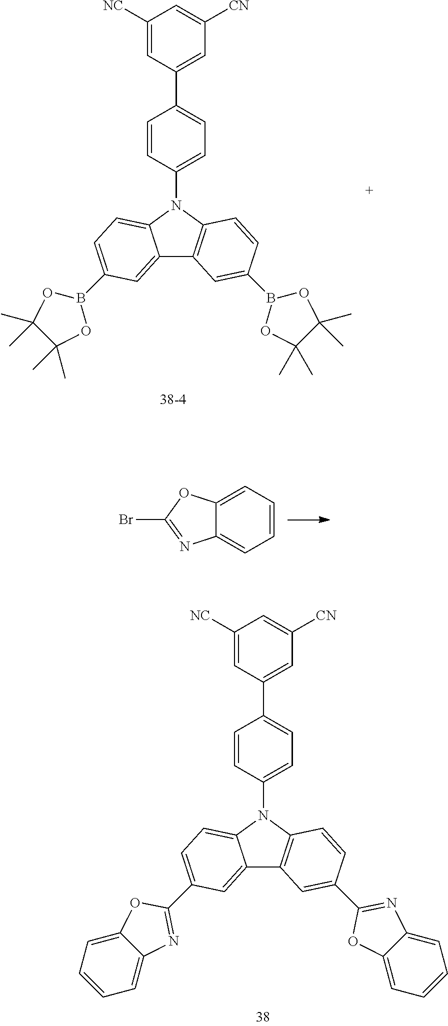

100 mL of toluene, 25 mL of EtOH, and 25 mL of H₂O were added to Intermediate 38-4 (10.0 g, 0.016 mol), 2-bromobenzoxazole (7.65 g, 0.039 mol), K₂CO₃ (13.35 g, 0.097 mol), and Pd(PPh₃)₄ (0.37 g, 0.3 mmol), and the resulting mixture was stirred under reflux at 100° C. for 6 hours and reacted. After completion of the reaction, the resulting product was extracted and concentrated, and then subjected to column chromatography and recrystallization to obtain 6.2 g (yield 63.8%) of <Compound 38>. LC/MS: m/z=603[(M+1)⁺]

### Synthesis Example 8: Synthesis of Compound 43

### (1) Preparation Example 1: Synthesis of Intermediate 43-1

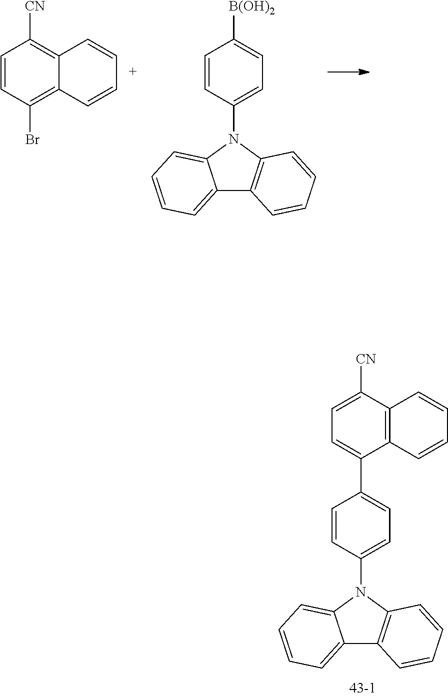 [SMILES: N#Cc1ccc(Br)c2ccccc12 ; OB(O)c1ccc(-n2c3ccccc3c3ccccc32)cc1 ; N#Cc1ccc(-c2ccc(-n3c4ccccc4c4ccccc43)cc2)c2ccccc12]

220 mL of toluene, 55 mL of EtOH, and 55 mL of H₂O were added to 4-bromo-1-naphthonitrile (10.0 g, 0.043 mol), 4-(9H-carbazol-9-yl)phenylboronic acid (14.85 g, 0.052 mol), K₂CO₃ (17.87 g, 0.129 mol), and Pd(PPh₃)₄ (1.00 g, 0.9 mmol), and the resulting mixture was stirred under reflux at 100° C. for 6 hours and reacted. After completion of the reaction, the resulting product was extracted and concentrated, and then subjected to column chromatography and recrystallization to obtain 13.5 g (yield 79.4%) of <Intermediate 43-1>.

### (2) Preparation Example 2: Synthesis of Intermediate 43-2

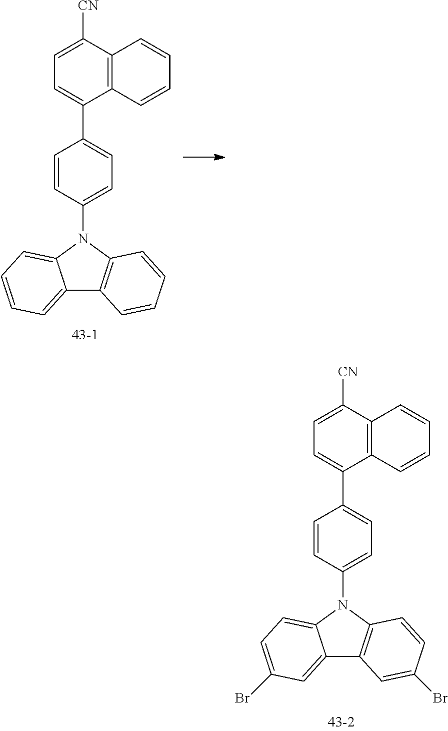 [SMILES: N#Cc1ccc(-c2ccc(-n3c4ccccc4c4ccccc43)cc2)c2ccccc12 ; N#Cc1ccc(-c2ccc(-n3c4ccc(Br)cc4c4cc(Br)ccc43)cc2)c2ccccc12]

130 mL of DMF was added to Intermediate 43-1 (10.0 g, 0.025 mol) and N-bromosuccinimide (10.8 g, 0.061 mol), and the resulting mixture was stirred under reflux at room temperature for 5 hours and reacted. After completion of the reaction, the resulting product was extracted and concentrated, and then subjected to column chromatography to obtain 9.2 g (yield 65.7%) of <Intermediate 43-2>.

### (3) Preparation Example 3: Synthesis of Intermediate 43-3

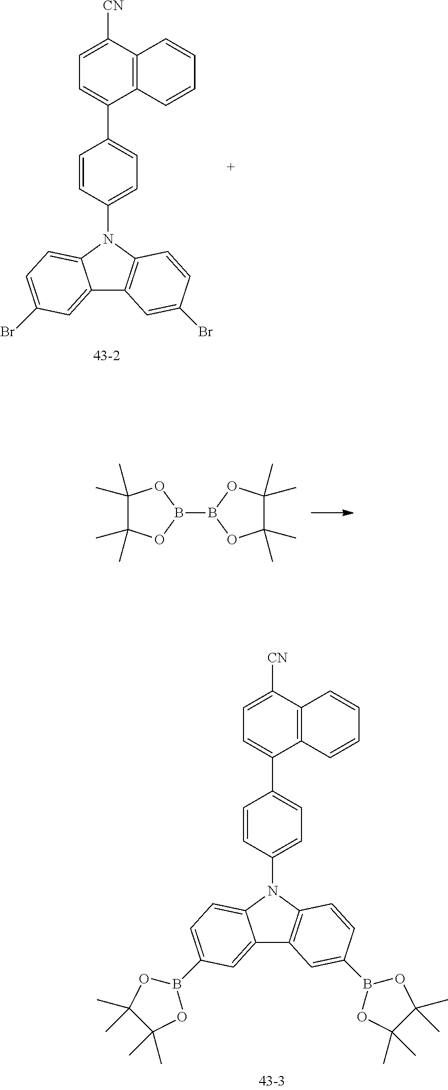

100 mL of dioxane was added to Intermediate 43-2 (10.0 g, 0.021 mol), bis(pinacolato)diboron (12.80 g, 0.050 mol), KOAc (8.24 g, 0.084 mol), and Pd(dppf)Cl₂ (0.92 g, 0.001 mol), and the resulting mixture was stirred at 100° C. for 12 hours and reacted. After completion of the reaction, the resulting product was extracted and concentrated, and then subjected to column chromatography and recrystallization to obtain 17.4 g (yield 76.2%) of <Intermediate 43-3>.

### (4) Preparation Example 4: Synthesis of Compound 43

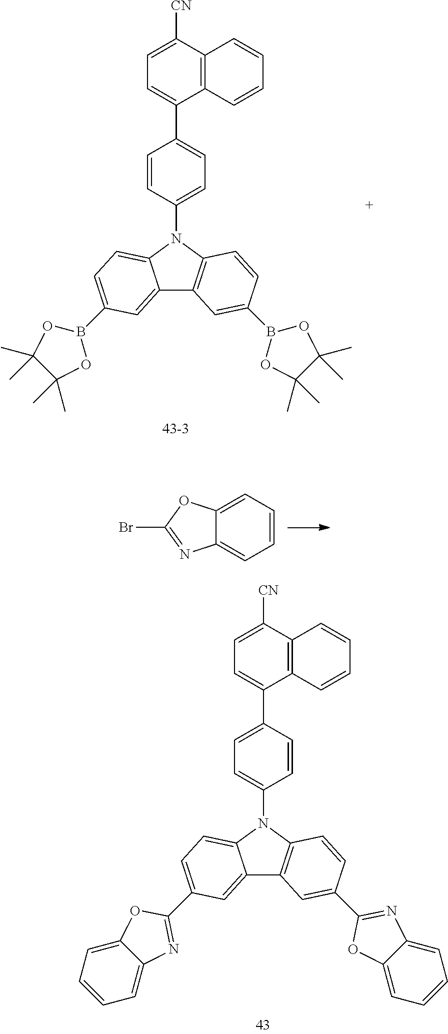

100 mL of toluene, 25 mL of EtOH, and 25 mL of H₂O were added to Intermediate 43-3 (10.0 g, 0.018 mol), 2-bromobenzoxazole (8.33 g, 0.042 mol), K₂CO₃ (14.54 g, 0.105 mol), and Pd(PPh₃)₄ (0.41 g, 0.4 mmol), and the resulting mixture was stirred under reflux at 100° C. for 6 hours and reacted. After completion of the reaction, the resulting product was extracted and concentrated, and then subjected to column chromatography and recrystallization to obtain 7.1 g (yield 73.3%) of <Compound 43>. LC/MS: m/z=628[(M+1)⁺]

### Synthesis Example 9: Synthesis of Compound 45

### (1) Preparation Example 1: Synthesis of Intermediate 45-1

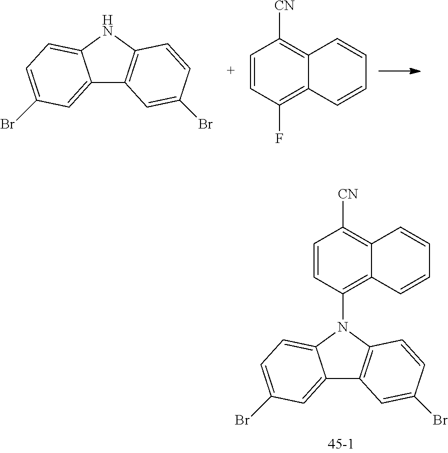 [SMILES: Brc1ccc2[nH]c3ccc(Br)cc3c2c1 ; N#Cc1ccc(F)c2ccccc12 ; N#Cc1ccc(-n2c3ccc(Br)cc3c3cc(Br)ccc32)c2ccccc12]

155 mL of DMF was added to 3,6-dibromocarbazole (10.0 g, 0.031 mol), 4-fluoro-1-naphthonitrile (6.32 g, 0.037 mol), and Cs₂CO₃ (6.38 g, 0.046 mol), and the resulting mixture was stirred under reflux at 150° C. for 12 hours and reacted. After completion of the reaction, the resulting product was extracted and concentrated, and then subjected to column chromatography to obtain 11.2 g (yield 76.4%) of <Intermediate 45-1>.

### (2) Preparation Example 2: Synthesis of Intermediate 45-2

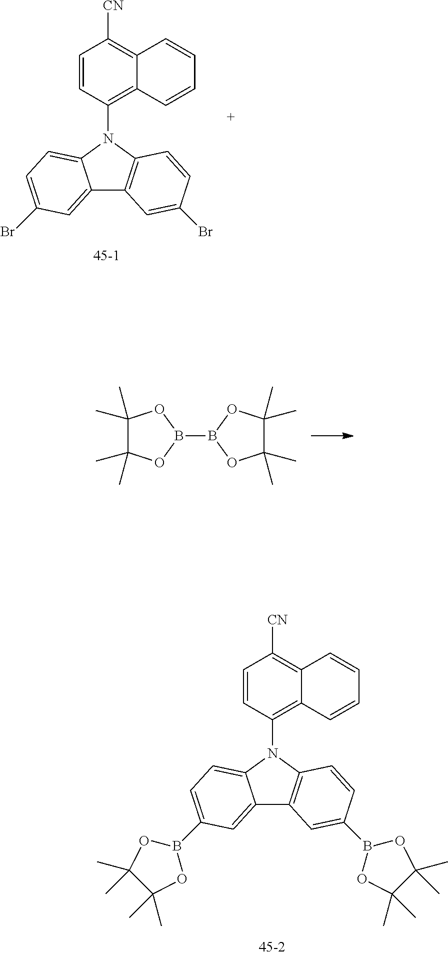

100 mL of dioxane was added to Intermediate 45-1 (10.0 g, 0.021 mol), bis(pinacolato)diboron (12.80 g, 0.050 mol), KOAc (8.24 g, 0.084 mol), and Pd(dppf)Cl₂ (0.92 g, 0.001 mol), and the resulting mixture was stirred at 100° C. for 12 hours and reacted. After completion of the reaction, the resulting product was extracted and concentrated, and then subjected to column chromatography and recrystallization to obtain 8.6 g (yield 71.8%) of <Intermediate 45-2>.

### (3) Preparation Example 3: Synthesis of Compound 45

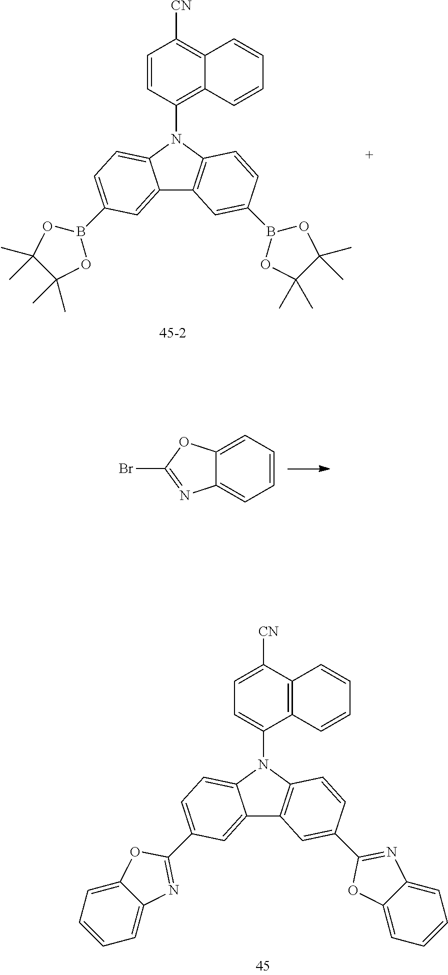

100 mL of toluene, 25 mL of EtOH, and 25 mL of H₂O were added to Intermediate 45-2 (10.0 g, 0.018 mol), 2-bromobenzoxazole (8.33 g, 0.042 mol), K₂CO₃ (14.54 g, 0.105 mol), and Pd(PPh₃)₄ (0.41 g, 0.4 mmol), and the resulting mixture was stirred under reflux at 100° C. for 6 hours and reacted. After completion of the reaction, the resulting product was extracted and concentrated, and then subjected to column chromatography and recrystallization to obtain 5.9 g (yield 61.0%) of <Compound 45>. LC/MS: m/z=552[(M+1)⁺]

### Synthesis Example 10: Synthesis of Compound 55

### (1) Preparation Example 1: Synthesis of Intermediate 55-1

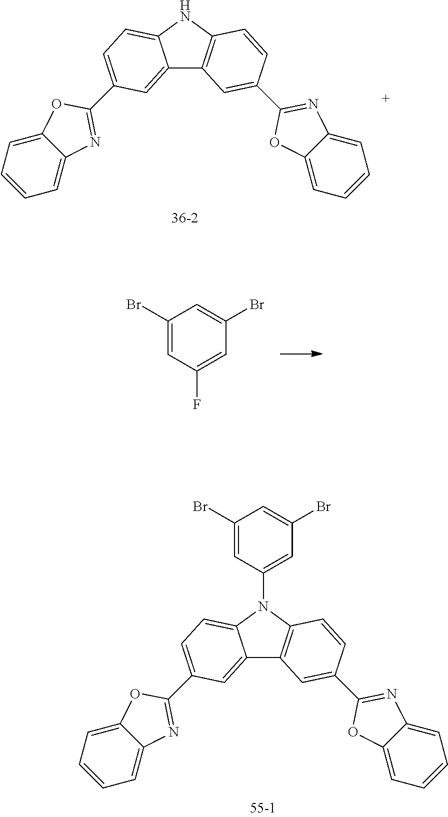 [SMILES: c1ccc2oc(-c3ccc4[nH]c5ccc(-c6nc7ccccc7o6)cc5c4c3)nc2c1 ; Fc1cc(Br)cc(Br)c1 ; Brc1cc(Br)cc(-n2c3ccc(-c4nc5ccccc5o4)cc3c3cc(-c4nc5ccccc5o4)ccc32)c1]

130 mL of DMF was added to Intermediate 35-2 (10.0 g, 0.025 mol), 1,3-dibromo-5-fluorobenzene (7.59 g, 0.030 mol), and Cs₂CO₃ (5.16 g, 0.037 mol), and the resulting mixture was stirred under reflux at 150° C. for 12 hours and reacted. After completion of the reaction, the resulting product was extracted and concentrated, and then subjected to column chromatography to obtain 12.1 g (yield 75.8%) of <Intermediate 55-1>.

### (2) Preparation Example 2: Synthesis of Compound 55

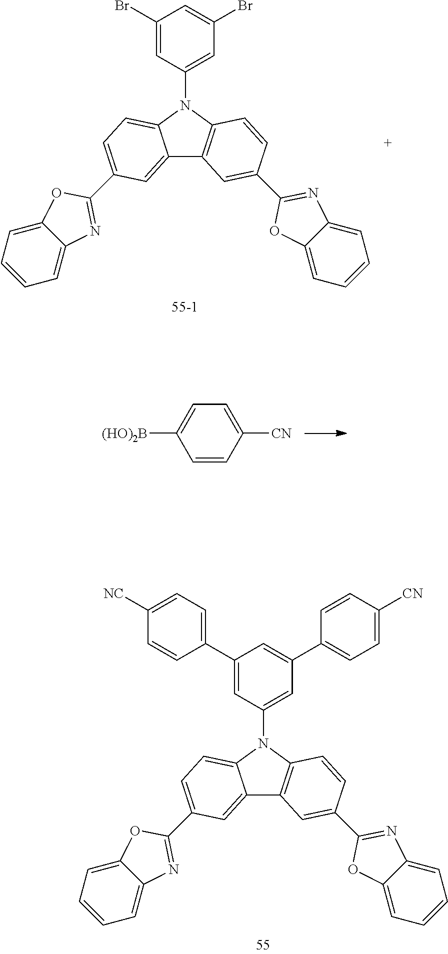 [SMILES: Brc1cc(Br)cc(-n2c3ccc(-c4nc5ccccc5o4)cc3c3cc(-c4nc5ccccc5o4)ccc32)c1 ; N#Cc1ccc(B(O)O)cc1 ; N#Cc1ccc(-c2cc(-c3ccc(C#N)cc3)cc(-n3c4ccc(-c5nc6ccccc6o5)cc4c4cc(-c5nc6ccccc6o5)ccc43)c2)cc1]

80 mL of toluene, 20 mL of EtOH, and 20 mL of H₂O were added to Intermediate 55-1 (10.0 g, 0.016 mol), 4-cyanophenylboronic acid (5.55 g, 0.038 mol), K₂CO₃ (13.05 g, 0.094 mol), and Pd(PPh₃)₄ (0.36 g, 0.3 mmol), and the resulting mixture was stirred under reflux at 100° C. for 6 hours and reacted. After completion of the reaction, the resulting product was extracted and concentrated, and then subjected to column chromatography and recrystallization to obtain 6.8 g (yield 63.6%) of <Compound 55>.

LC/MS: m/z=679[(M+1)⁺]

### Synthesis Example 11: Synthesis of Compound 65

### (1) Preparation Example 1: Synthesis of Compound 65

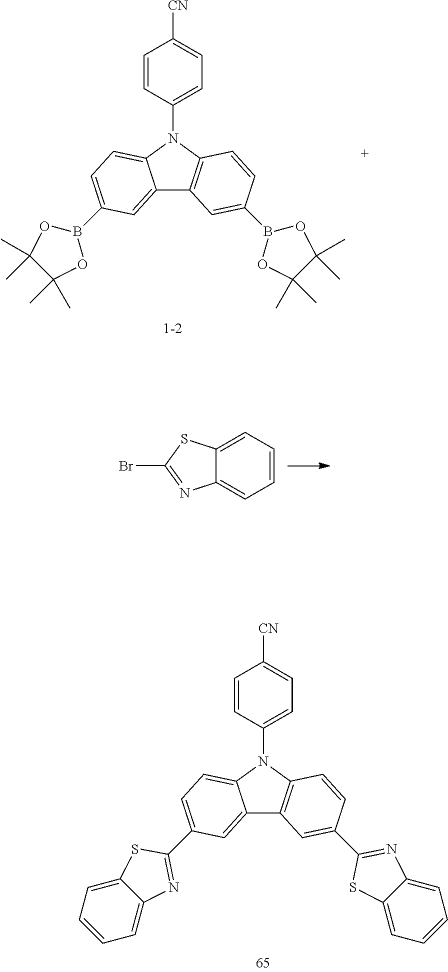

100 mL of toluene, 25 mL of EtOH, and 25 mL of H₂O were added to Intermediate 1-2 (10.0 g, 0.019 mol), 2-bromobenzothiazole (9.88 g, 0.046 mol), K₂CO₃ (15.94 g, 0.115 mol), and Pd(PPh₃)₄ (0.44 g, 0.4 mmol), and the resulting mixture was stirred under reflux at 100° C. for 6 hours and reacted. After completion of the reaction, the resulting product was extracted and concentrated, and then subjected to column chromatography and recrystallization to obtain 7.2 g (yield 70.0%) of <Compound 65>.

LC/MS: m/z=534[(M+1)⁺]

### Device Examples (Capping Layer)

In embodiments according to the present invention, an anode was patterned using an ITO glass substrate including Ag of 25 mm×25 mm×0.7 mm such that a light emitting area had a size of 2 mm×2 mm, and then washed. After the patterned ITO substrate was mounted in a vacuum chamber, an organic material and a metal were deposited on the substrate at a process pressure of 1×10⁻⁶ torr or more as the following structure.

### Device Examples 1 to 20

After a blue organic light emitting device having the following device structure was manufactured by employing a compound implemented by the present invention for a light efficiency improving layer, light emitting and driving characteristics according to the compound implemented according to the present invention were measured.

Ag/ITO/hole injection layer (HAT-CN, 5 nm)/hole transport layer (α-NPB, 100 nm)/electron blocking layer (TCTA, 10 nm)/light emitting layer (20 nm)/electron transport layer (201:Liq, 30 nm)/LiF (1 nm)/Mg:Ag (15 nm)/light efficiency improving layer (70 nm)

After [HAT-CN] was film-formed to a thickness of 5 nm on an ITO transparent electrode containing Ag on a glass substrate to form a hole injection layer, [α-NPB] was film-formed to 100 nm to form a hole transport layer, [TCTA] was film-formed to a thickness of 10 nm to form an electron blocking layer, [BH1] as a host compound and [BD1] as a dopant compound were used and co-deposited to 20 nm to form a light emitting layer, an electron transport layer (doped with 50% of the following [201] compound Liq) was deposited to a thickness of 30 nm, and then LiF was film-formed to a thickness of 1 nm to form an electron injection layer, Mg and Ag were film-formed to a thickness of 15 nm at a ratio of 1:9 to form a cathode, and a compound implemented by the present invention shown in the following [Table 1] was film-formed to a thickness of 70 nm to form a light efficiency improving layer (capping layer), thereby manufacturing an organic light emitting device.

### Device Comparative Example 1

An organic light emitting device for Device Comparative Example 1 was manufactured in the same manner as in the device structure in Example 1, except that the light efficiency improving layer was not used.

### Device Comparative Example 2

An organic light emitting device for Device Comparative Example 2 was manufactured in the same manner as in the device structure in Example 1, except that as the light efficiency improving layer compound, Alq₃ was used instead of the compound of the present invention.

### Device Comparative Example 3

An organic light emitting device for Device Comparative Example 3 was manufactured in the same manner as in the device structure in Example 1, except that as the light efficiency improving layer compound, the following CP1 was used instead of the compound of the present invention.

### Device Comparative Example 4

An organic light emitting device for Device Comparative Example 4 was manufactured in the same manner as in the device structure in Example 1, except that as the light efficiency improving layer compound, the following CP2 was used instead of the compound of the present invention.

### Experimental Example 1: Light Emitting Characteristics of Device Examples 1 to 20

For the organic light emitting devices manufactured by the Examples and the Comparative Examples, driving voltage, current efficiency and color coordinate were measured using a source meter (Mode1237, Keithley) and a luminance meter (PR-650, Photo Research), and the result values based on 1,000 nits are shown in the following [Table 1].

TABLE 1

| | Light efficiency | | | | |
|---|---|---|---|---|---|
| Example | improving layer | V | cd/A | CIEx | CIEy |
| 1 | Formula 1 | 3.7 | 8.5 | 0.139 | 0.051 |
| 2 | Formula 16 | 3.8 | 8.1 | 0.140 | 0.050 |
| 3 | Formula 21 | 3.6 | 8.8 | 0.143 | 0.048 |
| 4 | Formula 26 | 3.5 | 8.9 | 0.139 | 0.054 |
| 5 | Formula 30 | 3.7 | 8.5 | 0.138 | 0.053 |
| 6 | Formula 31 | 3.5 | 8.9 | 0.142 | 0.051 |
| 7 | Formula 35 | 3.7 | 8.3 | 0.143 | 0.048 |
| 8 | Formula 43 | 3.6 | 8.7 | 0.141 | 0.053 |
| 9 | Formula 45 | 3.7 | 8.4 | 0.139 | 0.056 |
| 10 | Formula 55 | 3.6 | 8.8 | 0.141 | 0.051 |
| 11 | Formula 65 | 3.8 | 8.2 | 0.141 | 0.050 |
| 12 | Formula 80 | 3.7 | 8.6 | 0.143 | 0.048 |
| 13 | Formula 89 | 3.4 | 9.0 | 0.141 | 0.052 |
| 14 | Formula 91 | 3.7 | 8.5 | 0.138 | 0.053 |
| 15 | Formula 93 | 3.8 | 8.1 | 0.141 | 0.050 |
| 16 | Formula 94 | 3.6 | 8.8 | 0.140 | 0.049 |
| 17 | Formula 97 | 3.5 | 8.9 | 0.139 | 0.055 |
| 18 | Formula 103 | 3.7 | 8.5 | 0.140 | 0.048 |
| 19 | Chemical | 3.5 | 8.9 | 0.140 | 0.049 |
| | Formula 108 | | | | |
| 20 | Formula 119 | 3.7 | 8.3 | 0.141 | 0.049 |
| Comparative | Not used | 4.2 | 7.0 | 0.146 | 0.141 |
| Example 1 | | | | | |
| Comparative | Alq₃ | 4.0 | 7.5 | 0.144 | 0.062 |
| Example 2 | | | | | |
| Comparative | CP1 | 3.9 | 8.0 | 0.138 | 0.058 |
| Example 3 | | | | | |
| Comparative | CP2 | 3.9 | 7.8 | 0.135 | 0.061 |
| Example 4 | | | | | |

Referring to the results shown in [Table 1], it can be confirmed that when the compound according to the present invention is applied to a light efficiency improving layer in a device, the device has reduced driving voltage and improved current efficiency compared to a device to which a light efficiency improving layer in the related art is not applied and devices (Comparative Examples 1 to 4) in which compounds used as a material for a light efficiency improving layer in the related art and compounds in comparison with the structural feature of the compound according to the present invention are each employed.

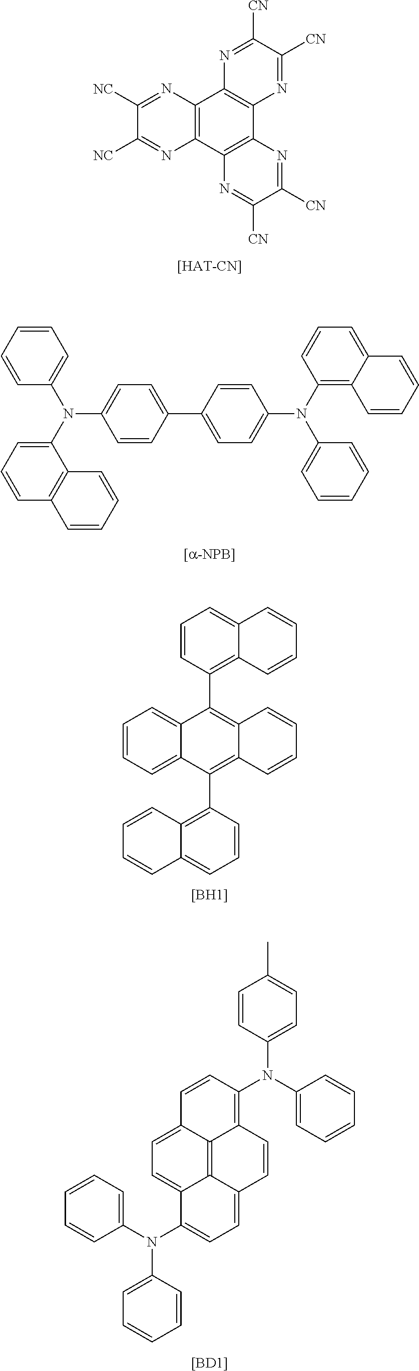

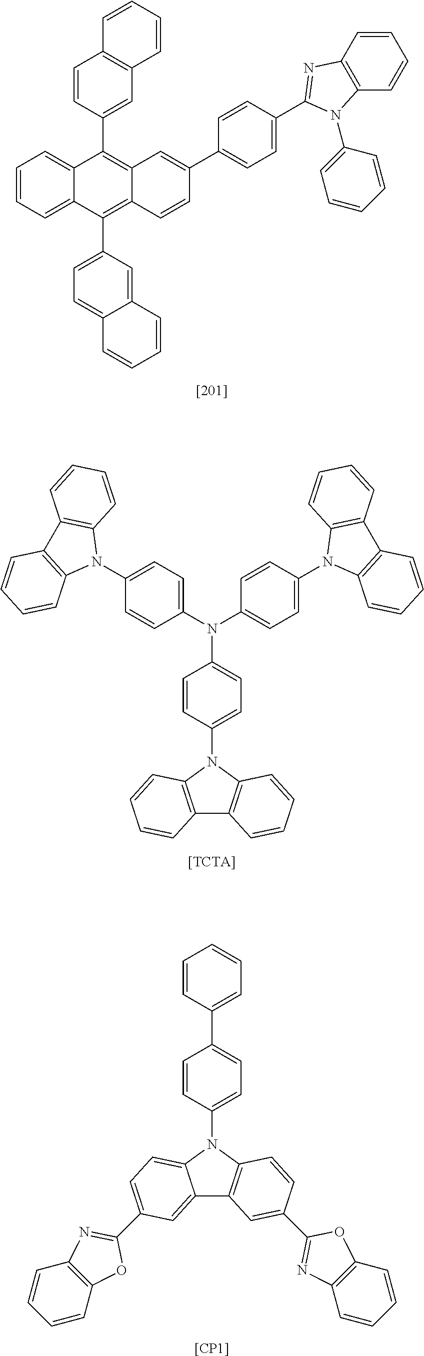

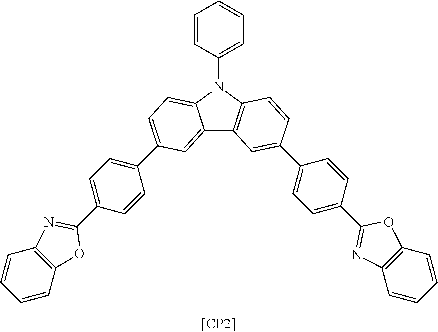

### Device Examples (ETL)

In exemplary embodiments according to the present invention, an ITO transparent electrode was patterned using an ITO glass substrate to which the ITO transparent electrode was attached on a glass substrate of 25 mm×25 mm×0.7 mm such that a light emitting area had a size of 2 mm×2 mm, and then washed. After the substrate was mounted in a vacuum chamber, a base pressure was set to 1×10⁻⁶ torr or more, and organic substances and a metal were deposited to have the following structure on the ITO.

### Device Examples 21 to 31

A compound implemented by the present invention was used as an electron transport layer. Luminescent properties including current efficiency were measured by manufacturing a blue organic light emitting device having the following device structure.

ITO/hole injection layer (HAT-CN, 5 nm)/hole transport layer (α-NPB, 100 nm)/electron blocking layer (EBL1 10 nm)/light emitting layer (20 nm)/hole blocking layer (HBL1, 50 nm)/electron transport layer (201:Liq, 30 nm)/LiF (1 nm)/Al (100 nm)

To form a hole injection layer on an ITO transparent electrode, [HAT-CN] was used and deposited to 5 nm, a hole transport layer was film-formed to 100 nm using α-NPB, an electron blocking layer was deposited to a thickness of 10 nm using [EBL1], and [BH1] as a host compound and [BD1] as a dopant compound were used and co-deposited to a thickness of 20 nm in a light emitting layer. Additionally, an electron transport layer was film-formed to a thickness of 30 nm (Liq doping) using a compound implemented by the present invention shown in the following [Table 2]. LiF and Al were film-formed to 1 nm and 100 nm, respectively, thereby manufacturing an organic light emitting device.

### Device Comparative Example 5

An organic light emitting device for Device Comparative Example 5 was manufactured in the same manner as in the device structure in Example 21, except that in the electron transport layer, the following was used instead of the compound implemented by the present invention.

### Device Comparative Example 6

An organic light emitting device for Device Comparative Example 6 was manufactured in the same manner as in the device structure in Example 21, except that in the electron transport layer, the following [ET1] was used instead of the compound implemented by the present invention.

### Device Comparative Example 7

An organic light emitting device for Device Comparative Example 7 was manufactured in the same manner as in the device structure in Example 21, except that in the electron transport layer, the following [ET2] was used instead of the compound implemented by the present invention.

### Experimental Example 2: Luminescent Properties of Device Examples 21 to 31

For the organic light emitting devices manufactured by the Examples and the Comparative Examples, voltage, current and luminous efficiency were measured using a source meter (Mode1237, Keithley) and a luminance meter (PR-650, Photo Research), and the result values based on 1000 nits are shown in the following [Table 2].

TABLE 2

| | Electron | | | | |
|---|---|---|---|---|---|
| Example | transport layer | V | cd/A | CIEx | CIEy |
| 21 | Formula 1 | 4.3 | 6.9 | 0.133 | 0.148 |
| 22 | Formula 16 | 4.5 | 7.2 | 0.132 | 0.149 |
| 23 | Formula 26 | 4.5 | 7.1 | 0.130 | 0.151 |
| 24 | Formula 30 | 4.5 | 7.1 | 0.131 | 0.151 |
| 25 | Formula 38 | 4.4 | 7.2 | 0.134 | 0.149 |
| 26 | Formula 65 | 4.5 | 7.2 | 0.132 | 0.148 |
| 27 | Formula 80 | 4.6 | 7.1 | 0.133 | 0.149 |
| 28 | Formula 89 | 4.4 | 7.3 | 0.134 | 0.152 |
| 29 | Formula 93 | 4.4 | 7.2 | 0.131 | 0.152 |
| 30 | Formula 94 | 4.2 | 7.0 | 0.132 | 0.150 |
| 31 | Formula 97 | 4.5 | 7.0 | 0.131 | 0.148 |
| Comparative | 201 | 4.7 | 6.6 | 0.135 | 0.151 |
| Example 5 | | | | | |
| Comparative | ET1 | 5.7 | 4.9 | 0.135 | 0.130 |
| Example 6 | | | | | |
| Comparative | ET2 | 5.8 | 4.8 | 0.133 | 0.133 |
| Example 7 | | | | | |

Referring to the results shown in [Table 2] above, it can be confirmed that when the compound according to the present invention is applied to an electron transport layer in the device, luminescent properties such as low-voltage driving and luminous efficiency are remarkably excellent compared to devices (Comparative Examples 5 to 7) in which Compound used as a material for an electron transport layer in the related art and [ET1] and [ET2], which are different from the characteristic structure of the compound according to the present invention, are employed.

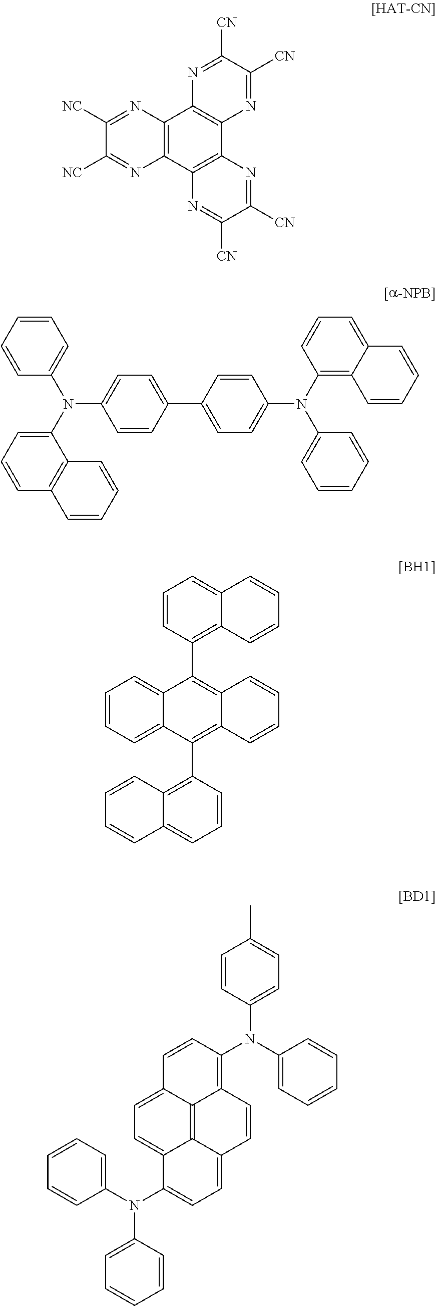

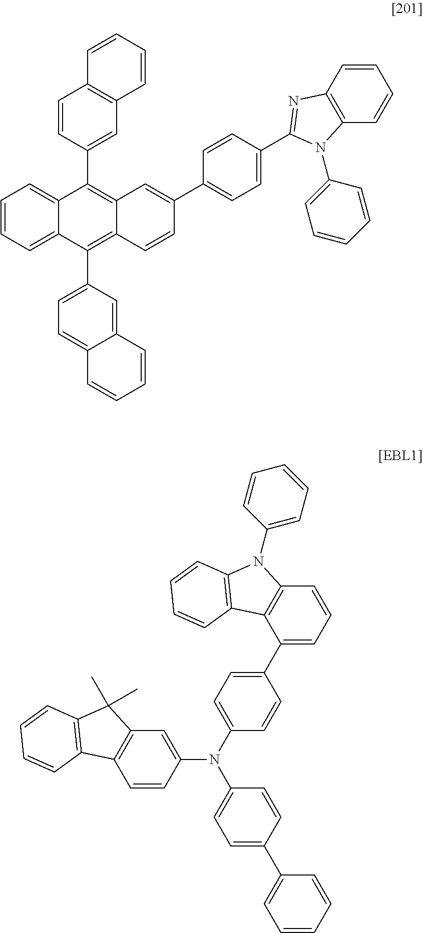 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; CC1(C)c2ccccc2-c2ccc(N(c3ccc(-c4ccccc4)cc3)c3ccc(-c4cccc5c4c4ccccc4n5-c4ccccc4)cc3)cc21]

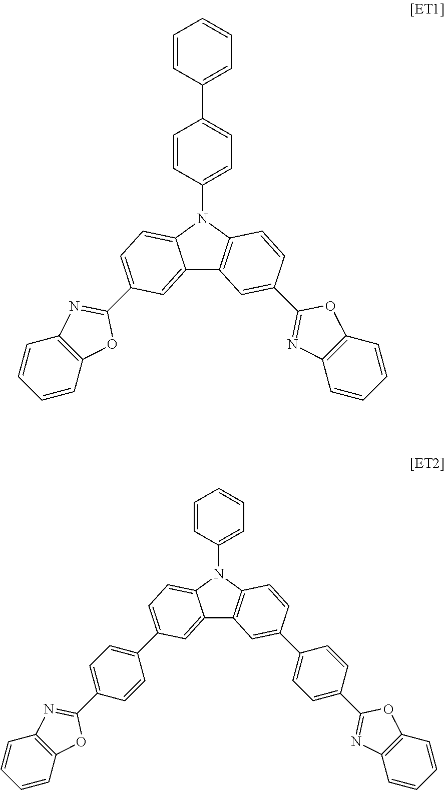 [SMILES: c1ccc(-c2ccc(-n3c4ccc(-c5nc6ccccc6o5)cc4c4cc(-c5nc6ccccc6o5)ccc43)cc2)cc1 ; c1ccc(-n2c3ccc(-c4ccc(-c5nc6ccccc6o5)cc4)cc3c3cc(-c4ccc(-c5nc6ccccc6o5)cc4)ccc32)cc1]

### INDUSTRIAL APPLICABILITY

When the organic compound according to the present invention is employed as a material for a light efficiency improving layer provided in an organic layer in an organic light emitting device or in an organic light emitting device, the organic compound can be industrially and usefully used for various lighting and display devices because it is possible to implement luminescent properties such as low-voltage driving of the device and excellent luminous efficiency.

1. An organic compound represented by Formula I below: 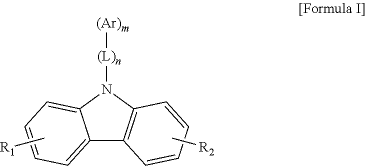
wherein in Formula I above,
Ar is a substituted or unsubstituted aryl group having 6 to 30 carbon atoms, in which at least one or more cyano groups (CN) are substituted (m is an integer of 1 or 2, and when m is 2, a plurality of Ar's are the same as or different from each other),
L is a single bond or a substituted or unsubstituted arylene group having 6 to 30 carbon atoms (n is an integer from 0 to 2, and when n is 2, a plurality of L's are the same as or different from each other),
R₁ to R₂ are each independently represented by Structural Formula 1 below, 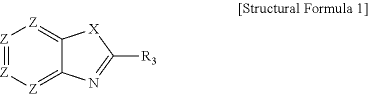
in Structural Formula 1 above,
X is O or S and Z is CR (a plurality of R's are the same as or different from each other),
R and R₃ are each independently selected from among hydrogen, deuterium, a halogen group, a cyano group, a substituted or unsubstituted alkyl group having 1 to 20 carbon atoms, a substituted or unsubstituted alkenyl group having 2 to 20 carbon atoms, a substituted or unsubstituted cycloalkyl group having 3 to 20 carbon atoms, a substituted or unsubstituted alkoxy group having 1 to 20 carbon atoms, a substituted or unsubstituted halogenated alkyl group having 1 to 20 carbon atoms, a substituted or unsubstituted halogenated alkoxy group having 1 to 20 carbon atoms, a substituted or unsubstituted aryl group having 6 to 30 carbon atoms and a substituted or unsubstituted heteroaryl group having 3 to 30 carbon atoms,
any one of the plurality of R's and R₃ is a moiety in which Structural Formula 1 above is each linked to Formula I above at the R₁ and R₂ positions, and
the plurality of R's and R₃ are optionally bonded to each other or linked to an adjacent substituent to form an aromatic monocyclic or polycyclic ring.

2. The organic compound of claim 1, wherein Structural Formula 1 above is any one selected from among structural formulae represented by Structural Formula 2 to Structural Formula 6 below: 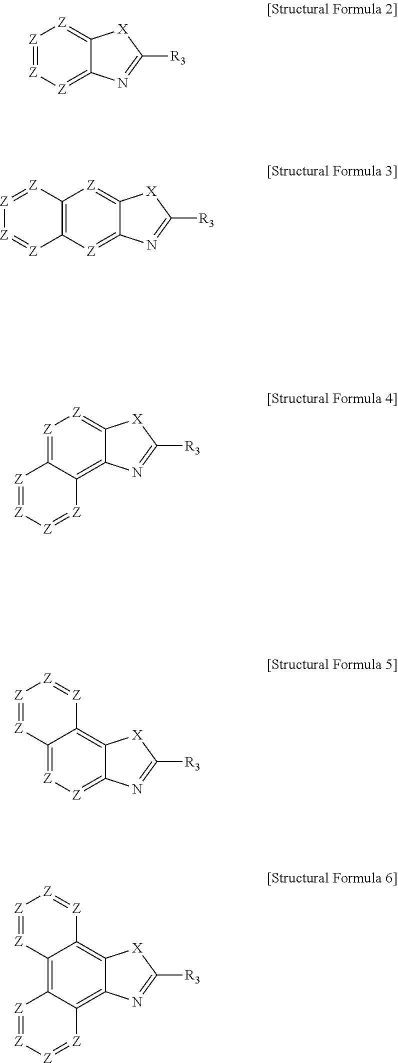
in Structural Formulae 2 to 6 above,
X is O or S and Z is CR (a plurality of R's are the same as or different from each other),
R and R₃ are each independently selected from among hydrogen, deuterium, a halogen group, a cyano group, a substituted or unsubstituted alkyl group having 1 to 20 carbon atoms, a substituted or unsubstituted alkenyl group having 2 to 20 carbon atoms, a substituted or unsubstituted cycloalkyl group having 3 to 20 carbon atoms, a substituted or unsubstituted alkoxy group having 1 to 20 carbon atoms, a substituted or unsubstituted halogenated alkyl group having 1 to 20 carbon atoms, a substituted or unsubstituted halogenated alkoxy group having 1 to 20 carbon atoms, a substituted or unsubstituted aryl group having 6 to 30 carbon atoms and a substituted or unsubstituted heteroaryl group having 3 to 30 carbon atoms, and
any one of the plurality of R's and R₃ is a moiety in which Structural Formula 1 above is each linked to Formula I above at the R₁ and R₂ positions.

3. The organic compound of claim 1, wherein in the definitions of Ar, L, R and R₃, the ‘substituted or unsubstituted’ means being substituted with one or two or more substituents selected from the group consisting of deuterium, a halogen group, a cyano group, a nitro group, a hydroxyl group, a silyl group, an alkyl group, a halogenated alkyl group, a deuterated alkyl group, a cycloalkyl group, a heterocycloalkyl group, an alkoxy group, a halogenated alkoxy group, a deuterated alkoxy group, an aryl group, a heteroaryl group, an alkylamine group, an arylamine group and a silyl group , being substituted with a substituent to which two or more substituent among the above-described substituents are linked, or having no substituent.

4. The organic compound of claim 1, wherein Formula I above is any one selected from among [Compound 1] to [Compound 134] below:

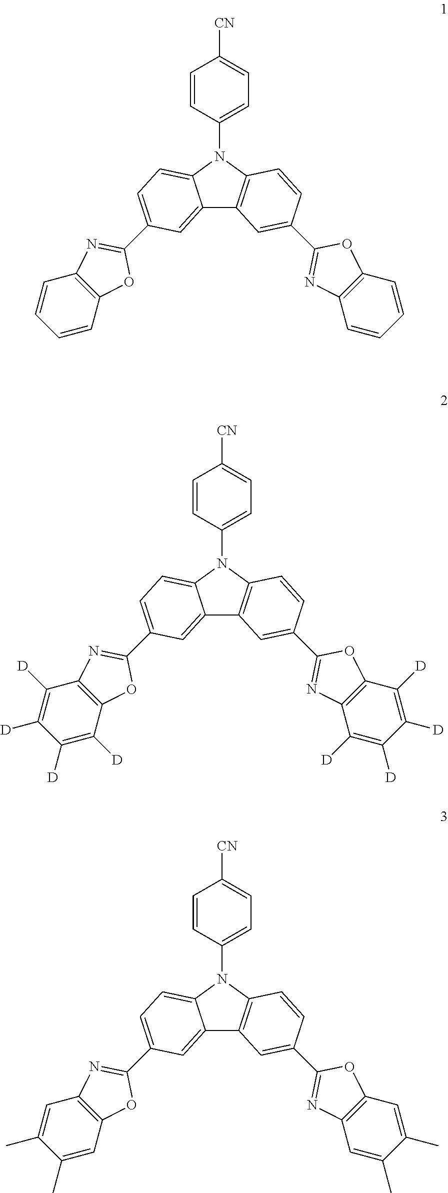

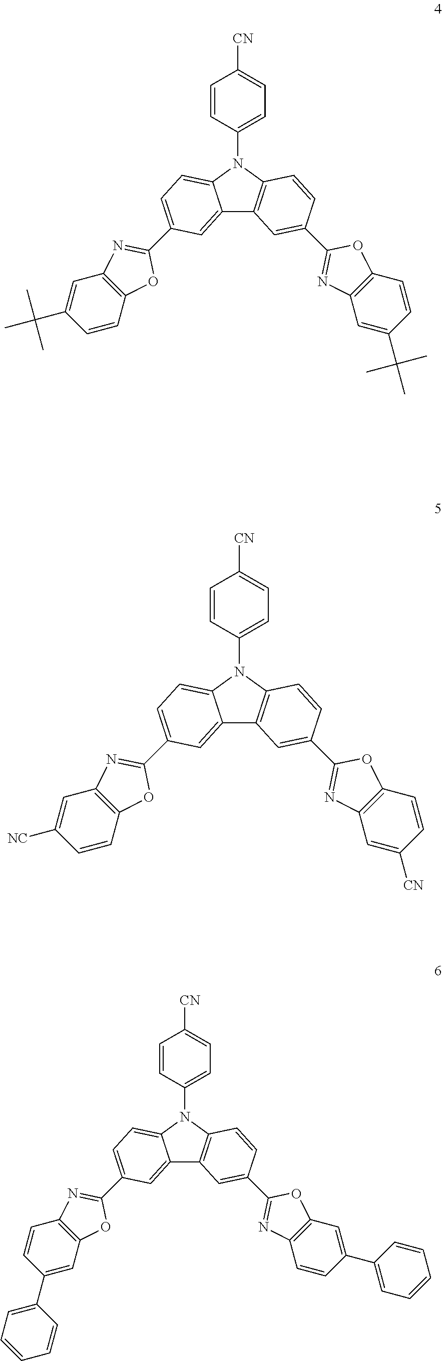 [SMILES: CC(C)(C)c1ccc2oc(-c3ccc4c(c3)c3cc(-c5nc6cc(C(C)(C)C)ccc6o5)ccc3n4-c3ccc(C#N)cc3)nc2c1 ; N#Cc1ccc(-n2c3ccc(-c4nc5cc(C#N)ccc5o4)cc3c3cc(-c4nc5cc(C#N)ccc5o4)ccc32)cc1 ; N#Cc1ccc(-n2c3ccc(-c4nc5ccc(-c6ccccc6)cc5o4)cc3c3cc(-c4nc5ccc(-c6ccccc6)cc5o4)ccc32)cc1]

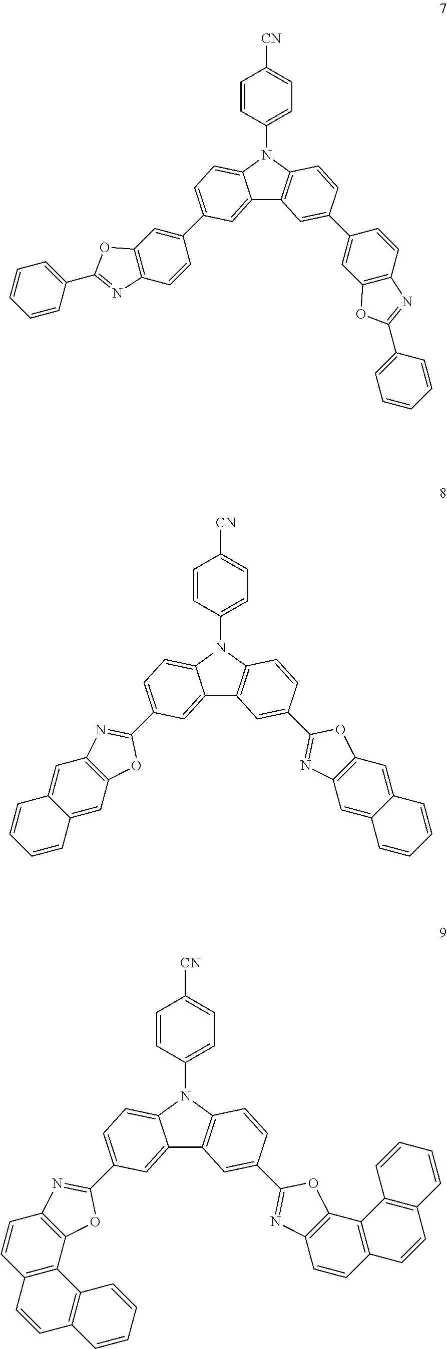 [SMILES: N#Cc1ccc(-n2c3ccc(-c4ccc5nc(-c6ccccc6)oc5c4)cc3c3cc(-c4ccc5nc(-c6ccccc6)oc5c4)ccc32)cc1 ; N#Cc1ccc(-n2c3ccc(-c4nc5cc6ccccc6cc5o4)cc3c3cc(-c4nc5cc6ccccc6cc5o4)ccc32)cc1 ; N#Cc1ccc(-n2c3ccc(-c4nc5ccc6ccc7ccccc7c6c5o4)cc3c3cc(-c4nc5ccc6ccc7ccccc7c6c5o4)ccc32)cc1]

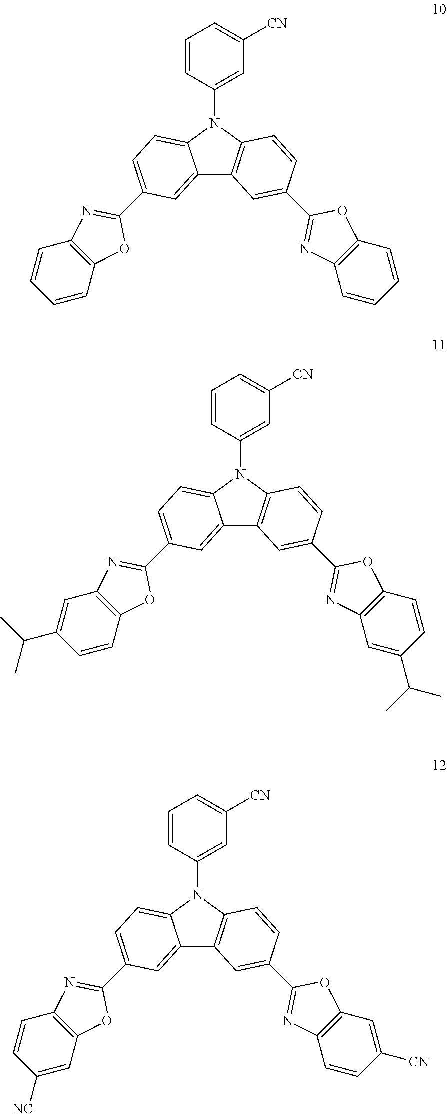 [SMILES: N#Cc1cccc(-n2c3ccc(-c4nc5ccccc5o4)cc3c3cc(-c4nc5ccccc5o4)ccc32)c1 ; CC(C)c1ccc2oc(-c3ccc4c(c3)c3cc(-c5nc6cc(C(C)C)ccc6o5)ccc3n4-c3cccc(C#N)c3)nc2c1 ; N#Cc1cccc(-n2c3ccc(-c4nc5ccc(C#N)cc5o4)cc3c3cc(-c4nc5ccc(C#N)cc5o4)ccc32)c1]

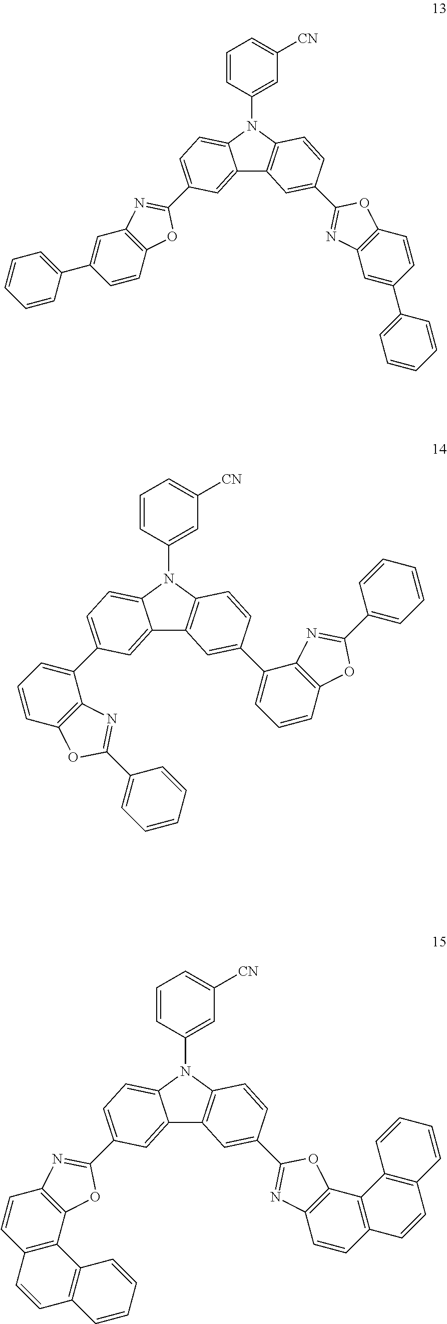 [SMILES: N#Cc1cccc(-n2c3ccc(-c4nc5cc(-c6ccccc6)ccc5o4)cc3c3cc(-c4nc5cc(-c6ccccc6)ccc5o4)ccc32)c1 ; N#Cc1cccc(-n2c3ccc(-c4cccc5oc(-c6ccccc6)nc45)cc3c3cc(-c4cccc5oc(-c6ccccc6)nc45)ccc32)c1 ; N#Cc1cccc(-n2c3ccc(-c4nc5ccc6ccc7ccccc7c6c5o4)cc3c3cc(-c4nc5ccc6ccc7ccccc7c6c5o4)ccc32)c1]

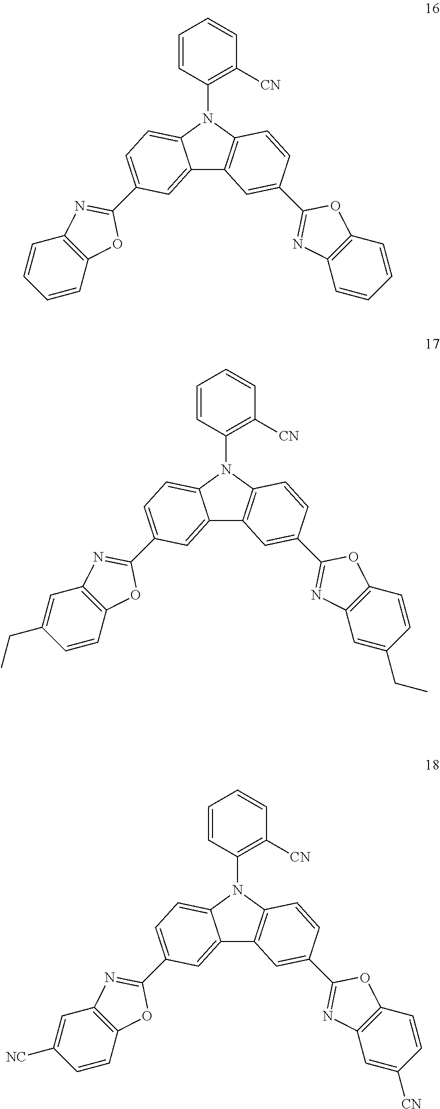 [SMILES: N#Cc1ccccc1-n1c2ccc(-c3nc4ccccc4o3)cc2c2cc(-c3nc4ccccc4o3)ccc21 ; CCc1ccc2oc(-c3ccc4c(c3)c3cc(-c5nc6cc(CC)ccc6o5)ccc3n4-c3ccccc3C#N)nc2c1 ; N#Cc1ccc2oc(-c3ccc4c(c3)c3cc(-c5nc6cc(C#N)ccc6o5)ccc3n4-c3ccccc3C#N)nc2c1]

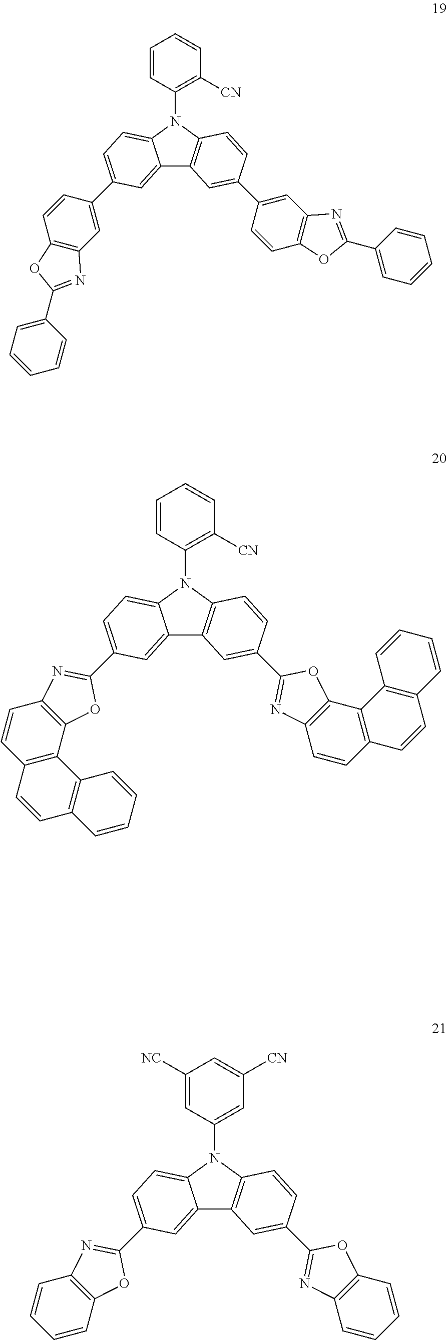 [SMILES: N#Cc1ccccc1-n1c2ccc(-c3ccc4oc(-c5ccccc5)nc4c3)cc2c2cc(-c3ccc4oc(-c5ccccc5)nc4c3)ccc21 ; N#Cc1ccccc1-n1c2ccc(-c3nc4ccc5ccc6ccccc6c5c4o3)cc2c2cc(-c3nc4ccc5ccc6ccccc6c5c4o3)ccc21 ; N#Cc1cc(C#N)cc(-n2c3ccc(-c4nc5ccccc5o4)cc3c3cc(-c4nc5ccccc5o4)ccc32)c1]

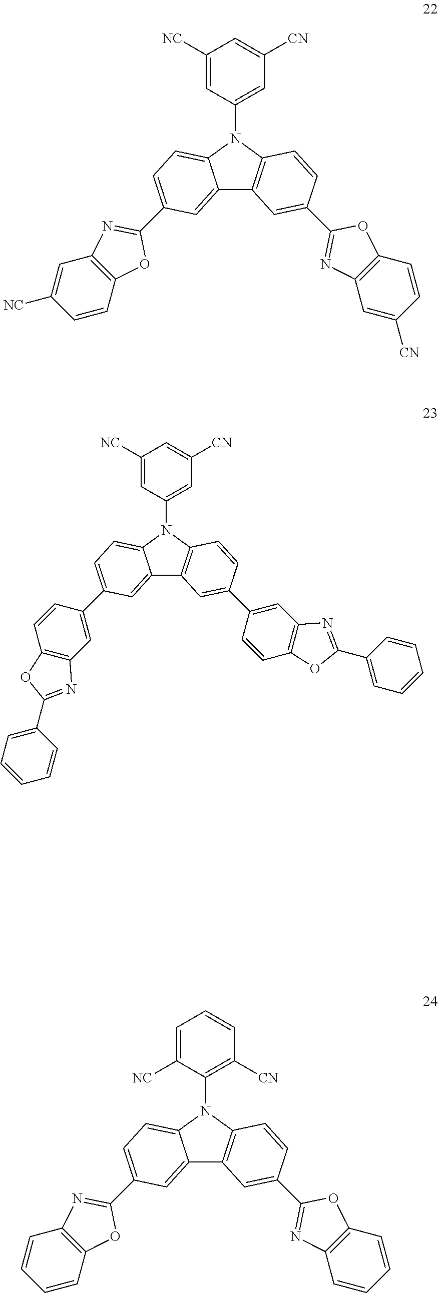 [SMILES: N#Cc1cc(C#N)cc(-n2c3ccc(-c4nc5cc(C#N)ccc5o4)cc3c3cc(-c4nc5cc(C#N)ccc5o4)ccc32)c1 ; N#Cc1cc(C#N)cc(-n2c3ccc(-c4ccc5oc(-c6ccccc6)nc5c4)cc3c3cc(-c4ccc5oc(-c6ccccc6)nc5c4)ccc32)c1 ; N#Cc1cccc(C#N)c1-n1c2ccc(-c3nc4ccccc4o3)cc2c2cc(-c3nc4ccccc4o3)ccc21]

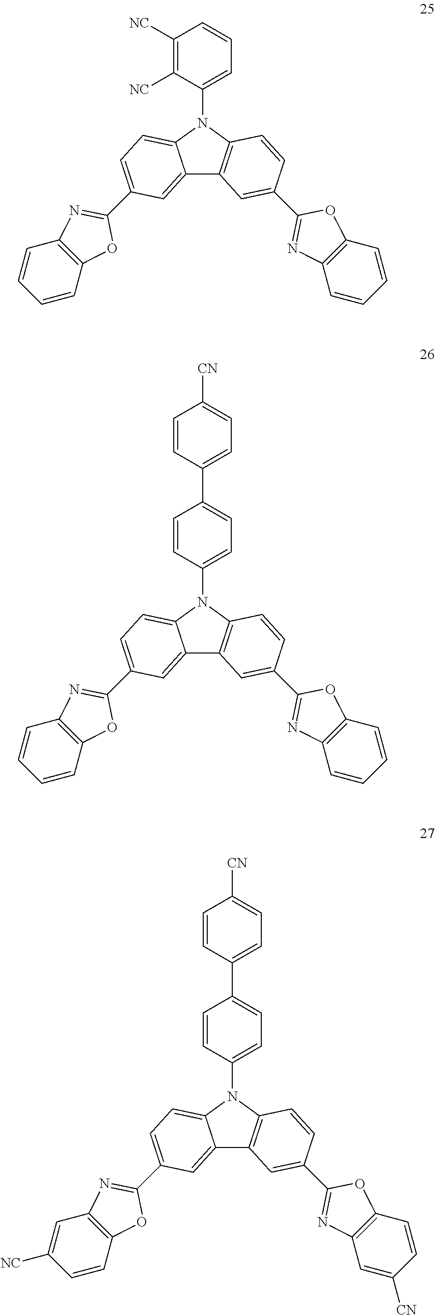 [SMILES: N#Cc1cccc(-n2c3ccc(-c4nc5ccccc5o4)cc3c3cc(-c4nc5ccccc5o4)ccc32)c1C#N ; N#Cc1ccc(-c2ccc(-n3c4ccc(-c5nc6ccccc6o5)cc4c4cc(-c5nc6ccccc6o5)ccc43)cc2)cc1 ; N#Cc1ccc(-c2ccc(-n3c4ccc(-c5nc6cc(C#N)ccc6o5)cc4c4cc(-c5nc6cc(C#N)ccc6o5)ccc43)cc2)cc1]

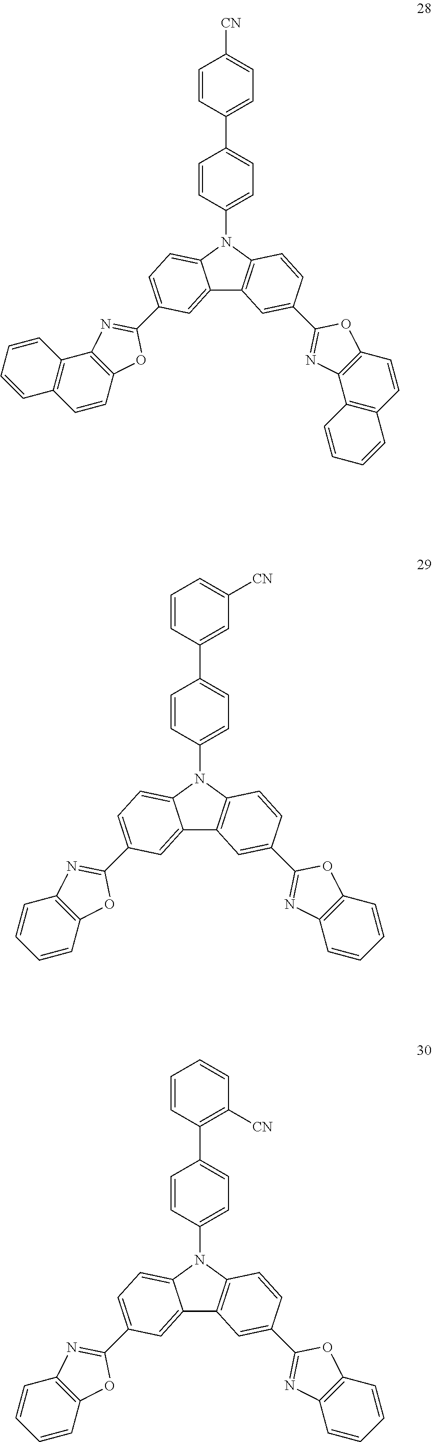 [SMILES: N#Cc1ccc(-c2ccc(-n3c4ccc(-c5nc6c(ccc7ccccc76)o5)cc4c4cc(-c5nc6c(ccc7ccccc76)o5)ccc43)cc2)cc1 ; N#Cc1cccc(-c2ccc(-n3c4ccc(-c5nc6ccccc6o5)cc4c4cc(-c5nc6ccccc6o5)ccc43)cc2)c1 ; N#Cc1ccccc1-c1ccc(-n2c3ccc(-c4nc5ccccc5o4)cc3c3cc(-c4nc5ccccc5o4)ccc32)cc1]

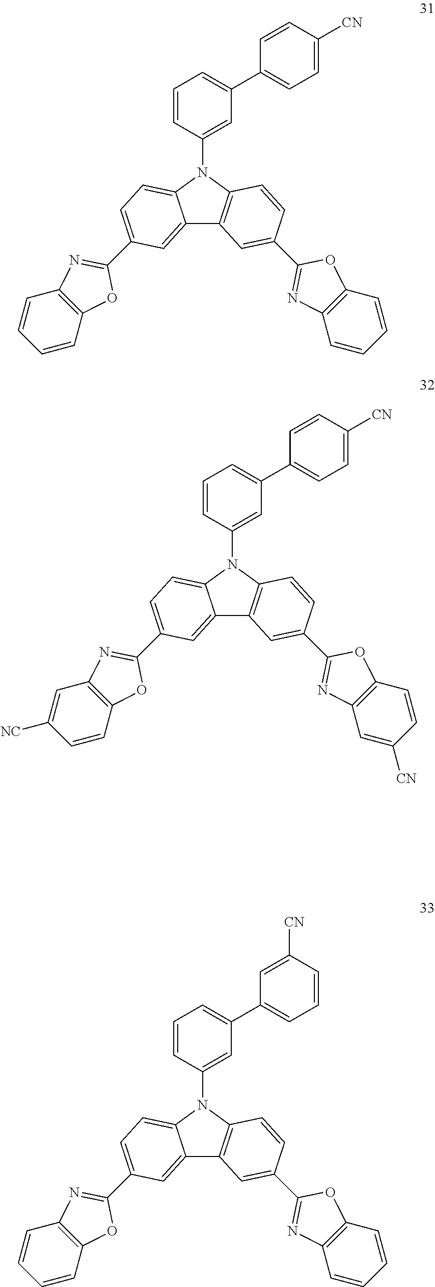 [SMILES: N#Cc1ccc(-c2cccc(-n3c4ccc(-c5nc6ccccc6o5)cc4c4cc(-c5nc6ccccc6o5)ccc43)c2)cc1 ; N#Cc1ccc(-c2cccc(-n3c4ccc(-c5nc6cc(C#N)ccc6o5)cc4c4cc(-c5nc6cc(C#N)ccc6o5)ccc43)c2)cc1 ; N#Cc1cccc(-c2cccc(-n3c4ccc(-c5nc6ccccc6o5)cc4c4cc(-c5nc6ccccc6o5)ccc43)c2)c1]

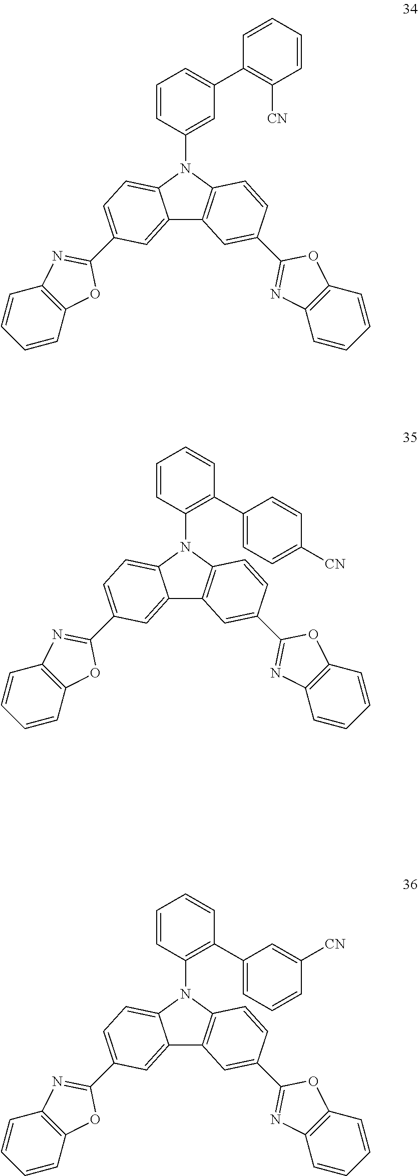 [SMILES: N#Cc1ccccc1-c1cccc(-n2c3ccc(-c4nc5ccccc5o4)cc3c3cc(-c4nc5ccccc5o4)ccc32)c1 ; N#Cc1ccc(-c2ccccc2-n2c3ccc(-c4nc5ccccc5o4)cc3c3cc(-c4nc5ccccc5o4)ccc32)cc1 ; N#Cc1cccc(-c2ccccc2-n2c3ccc(-c4nc5ccccc5o4)cc3c3cc(-c4nc5ccccc5o4)ccc32)c1]

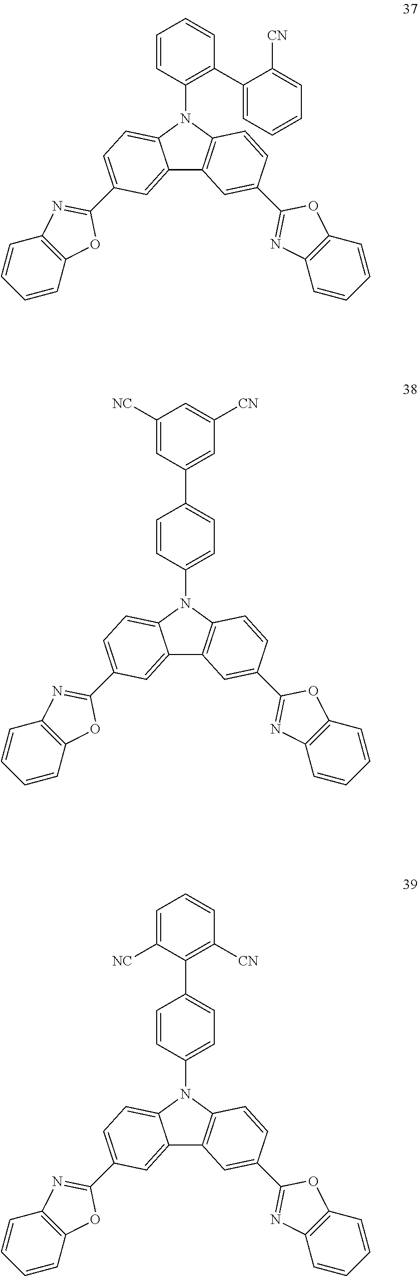 [SMILES: N#Cc1ccccc1-c1ccccc1-n1c2ccc(-c3nc4ccccc4o3)cc2c2cc(-c3nc4ccccc4o3)ccc21 ; N#Cc1cc(C#N)cc(-c2ccc(-n3c4ccc(-c5nc6ccccc6o5)cc4c4cc(-c5nc6ccccc6o5)ccc43)cc2)c1 ; N#Cc1cccc(C#N)c1-c1ccc(-n2c3ccc(-c4nc5ccccc5o4)cc3c3cc(-c4nc5ccccc5o4)ccc32)cc1]

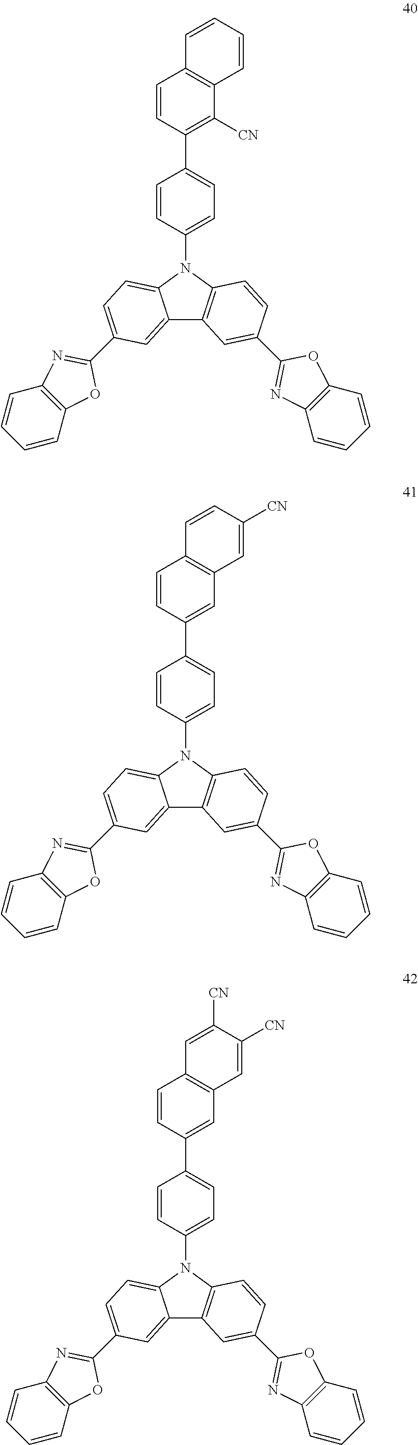 [SMILES: N#Cc1c(-c2ccc(-n3c4ccc(-c5nc6ccccc6o5)cc4c4cc(-c5nc6ccccc6o5)ccc43)cc2)ccc2ccccc12 ; N#Cc1ccc2ccc(-c3ccc(-n4c5ccc(-c6nc7ccccc7o6)cc5c5cc(-c6nc7ccccc7o6)ccc54)cc3)cc2c1 ; N#Cc1cc2ccc(-c3ccc(-n4c5ccc(-c6nc7ccccc7o6)cc5c5cc(-c6nc7ccccc7o6)ccc54)cc3)cc2cc1C#N]

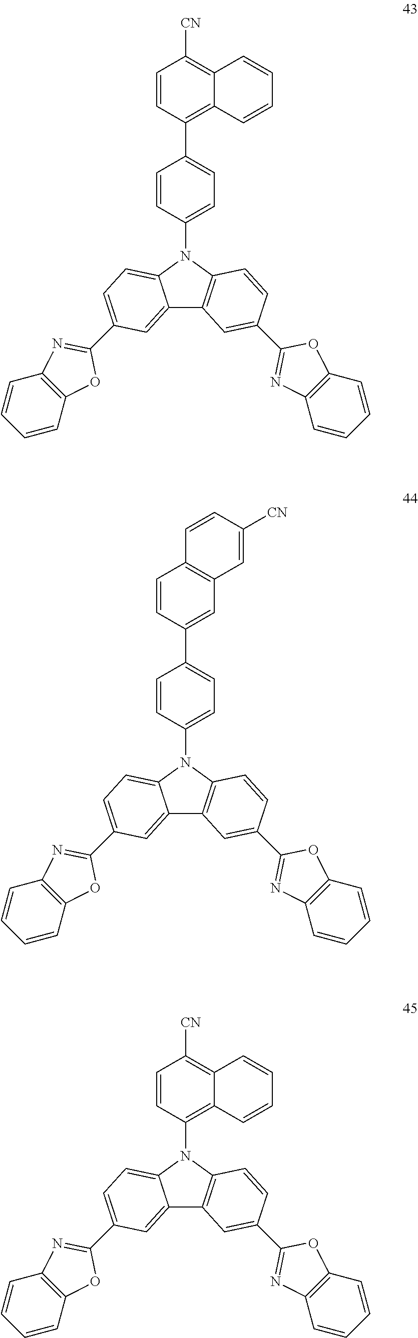 [SMILES: N#Cc1ccc(-c2ccc(-n3c4ccc(-c5nc6ccccc6o5)cc4c4cc(-c5nc6ccccc6o5)ccc43)cc2)c2ccccc12 ; N#Cc1ccc2ccc(-c3ccc(-n4c5ccc(-c6nc7ccccc7o6)cc5c5cc(-c6nc7ccccc7o6)ccc54)cc3)cc2c1 ; N#Cc1ccc(-n2c3ccc(-c4nc5ccccc5o4)cc3c3cc(-c4nc5ccccc5o4)ccc32)c2ccccc12]

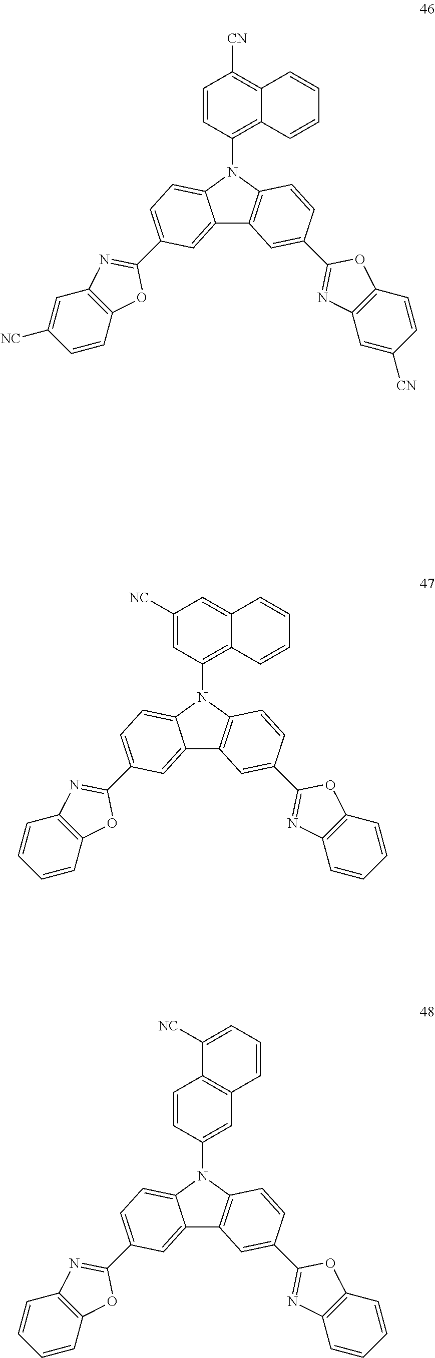 [SMILES: N#Cc1ccc2oc(-c3ccc4c(c3)c3cc(-c5nc6cc(C#N)ccc6o5)ccc3n4-c3ccc(C#N)c4ccccc34)nc2c1 ; N#Cc1cc(-n2c3ccc(-c4nc5ccccc5o4)cc3c3cc(-c4nc5ccccc5o4)ccc32)c2ccccc2c1 ; N#Cc1cccc2cc(-n3c4ccc(-c5nc6ccccc6o5)cc4c4cc(-c5nc6ccccc6o5)ccc43)ccc12]

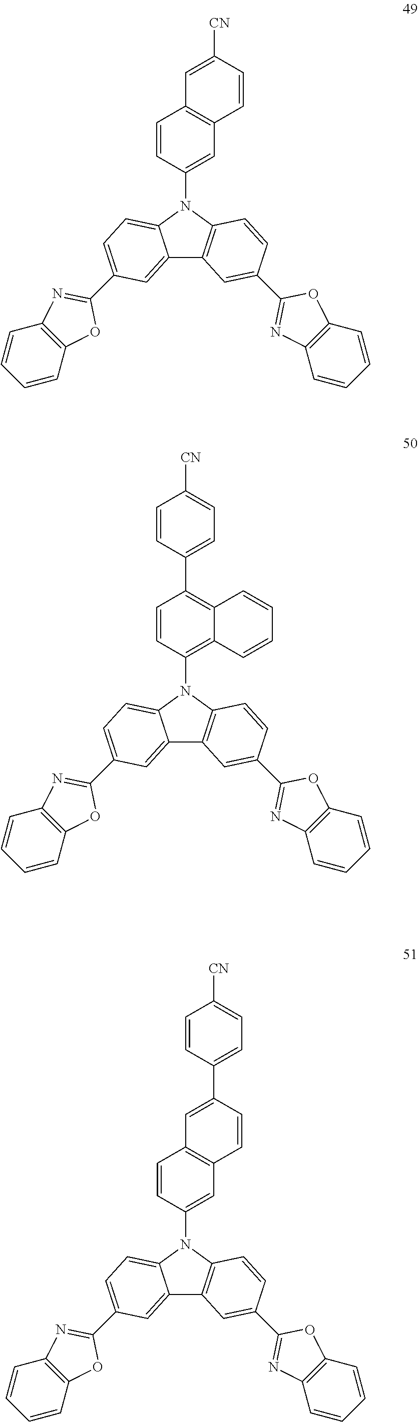 [SMILES: N#Cc1ccc2cc(-n3c4ccc(-c5nc6ccccc6o5)cc4c4cc(-c5nc6ccccc6o5)ccc43)ccc2c1 ; N#Cc1ccc(-c2ccc(-n3c4ccc(-c5nc6ccccc6o5)cc4c4cc(-c5nc6ccccc6o5)ccc43)c3ccccc23)cc1 ; N#Cc1ccc(-c2ccc3cc(-n4c5ccc(-c6nc7ccccc7o6)cc5c5cc(-c6nc7ccccc7o6)ccc54)ccc3c2)cc1]

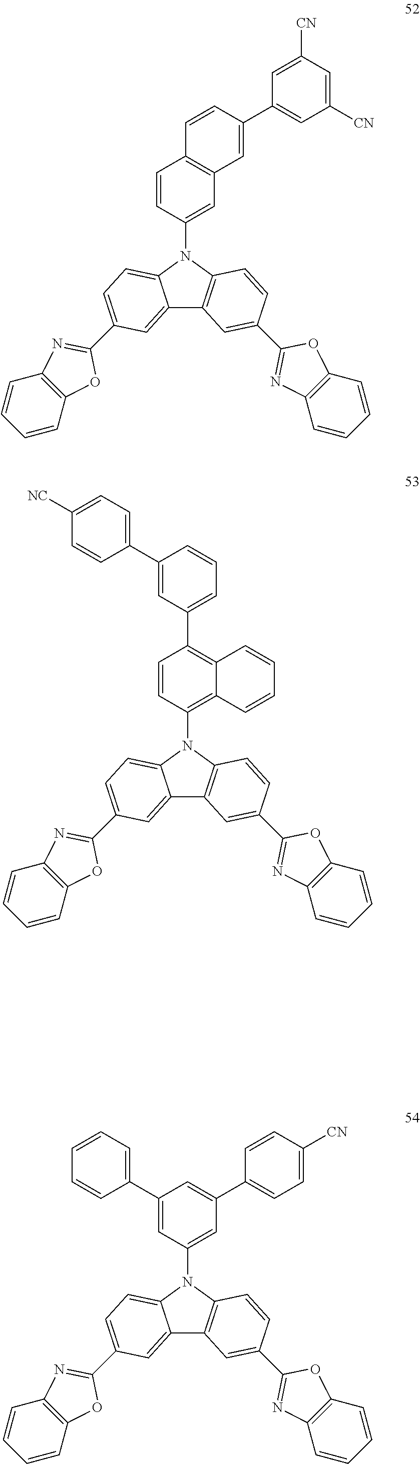 [SMILES: N#Cc1cc(C#N)cc(-c2ccc3ccc(-n4c5ccc(-c6nc7ccccc7o6)cc5c5cc(-c6nc7ccccc7o6)ccc54)cc3c2)c1 ; N#Cc1ccc(-c2cccc(-c3ccc(-n4c5ccc(-c6nc7ccccc7o6)cc5c5cc(-c6nc7ccccc7o6)ccc54)c4ccccc34)c2)cc1 ; N#Cc1ccc(-c2cc(-c3ccccc3)cc(-n3c4ccc(-c5nc6ccccc6o5)cc4c4cc(-c5nc6ccccc6o5)ccc43)c2)cc1]

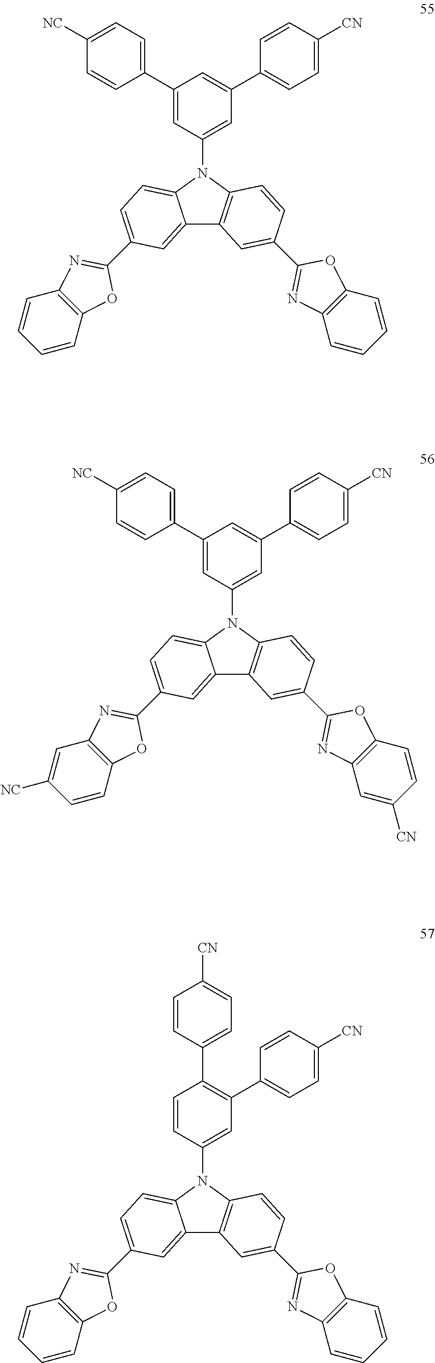 [SMILES: N#Cc1ccc(-c2cc(-c3ccc(C#N)cc3)cc(-n3c4ccc(-c5nc6ccccc6o5)cc4c4cc(-c5nc6ccccc6o5)ccc43)c2)cc1 ; N#Cc1ccc(-c2cc(-c3ccc(C#N)cc3)cc(-n3c4ccc(-c5nc6cc(C#N)ccc6o5)cc4c4cc(-c5nc6cc(C#N)ccc6o5)ccc43)c2)cc1 ; N#Cc1ccc(-c2ccc(-n3c4ccc(-c5nc6ccccc6o5)cc4c4cc(-c5nc6ccccc6o5)ccc43)cc2-c2ccc(C#N)cc2)cc1]

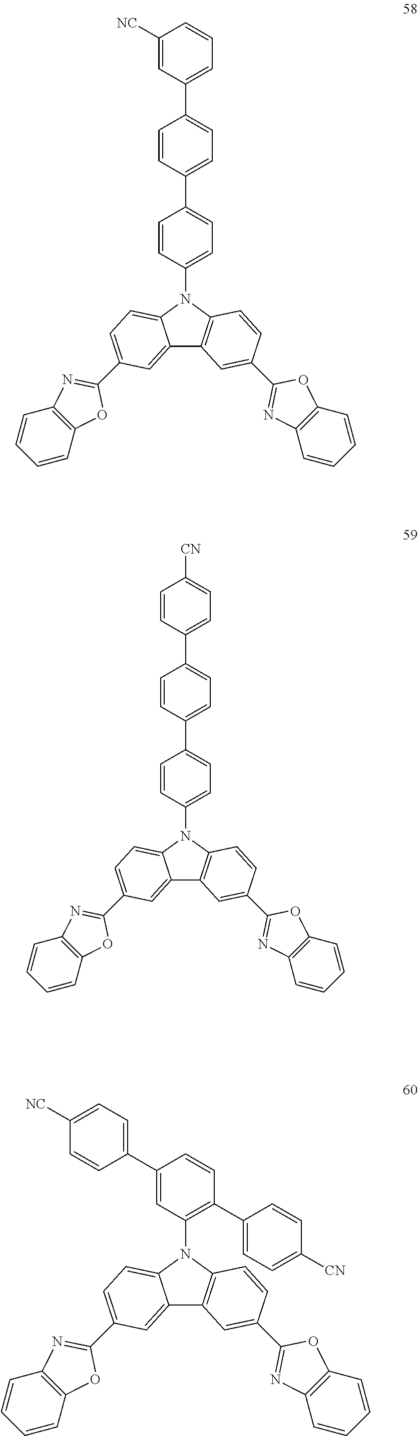 [SMILES: N#Cc1cccc(-c2ccc(-c3ccc(-n4c5ccc(-c6nc7ccccc7o6)cc5c5cc(-c6nc7ccccc7o6)ccc54)cc3)cc2)c1 ; N#Cc1ccc(-c2ccc(-c3ccc(-n4c5ccc(-c6nc7ccccc7o6)cc5c5cc(-c6nc7ccccc7o6)ccc54)cc3)cc2)cc1 ; N#Cc1ccc(-c2ccc(-c3ccc(C#N)cc3)c(-n3c4ccc(-c5nc6ccccc6o5)cc4c4cc(-c5nc6ccccc6o5)ccc43)c2)cc1]

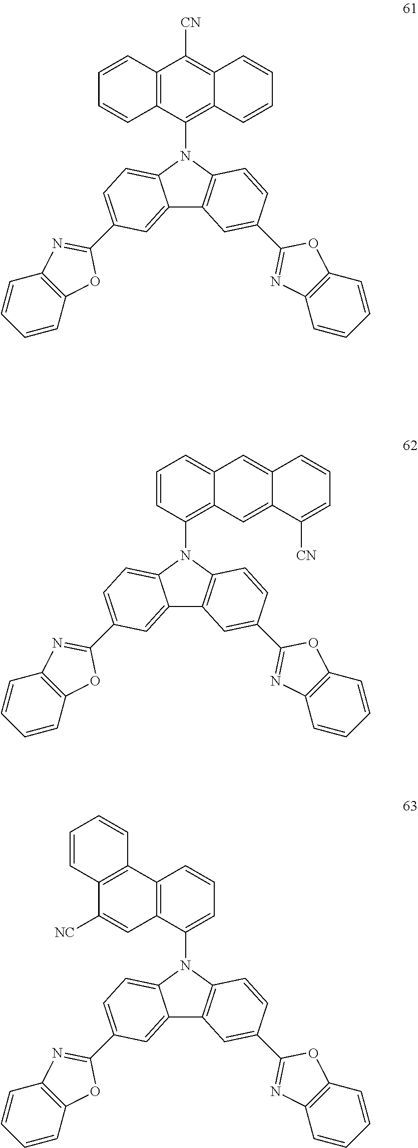 [SMILES: N#Cc1c2ccccc2c(-n2c3ccc(-c4nc5ccccc5o4)cc3c3cc(-c4nc5ccccc5o4)ccc32)c2ccccc12 ; N#Cc1cccc2cc3cccc(-n4c5ccc(-c6nc7ccccc7o6)cc5c5cc(-c6nc7ccccc7o6)ccc54)c3cc12 ; N#Cc1cc2c(-n3c4ccc(-c5nc6ccccc6o5)cc4c4cc(-c5nc6ccccc6o5)ccc43)cccc2c2ccccc12]

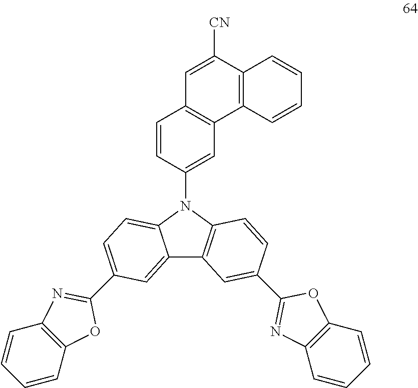 [SMILES: N#Cc1cc2ccc(-n3c4ccc(-c5nc6ccccc6o5)cc4c4cc(-c5nc6ccccc6o5)ccc43)cc2c2ccccc12]

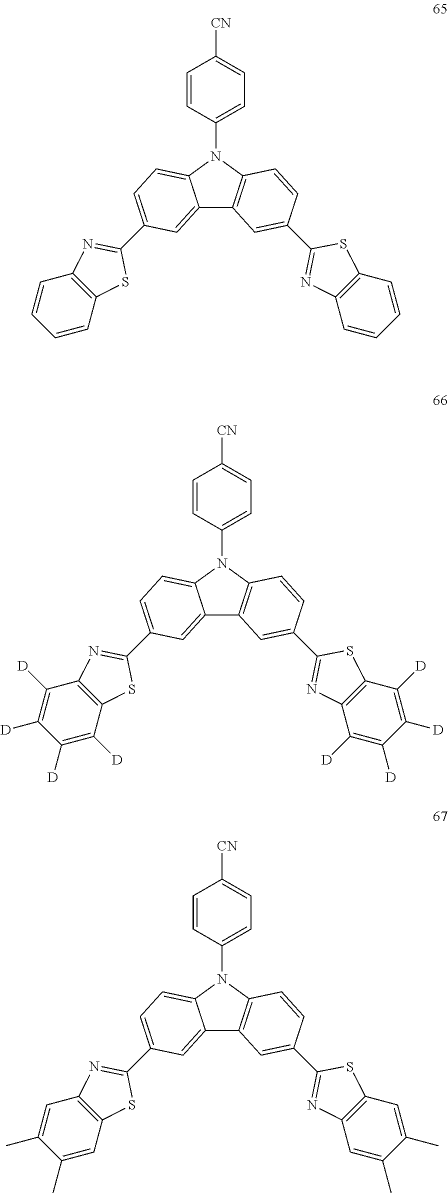

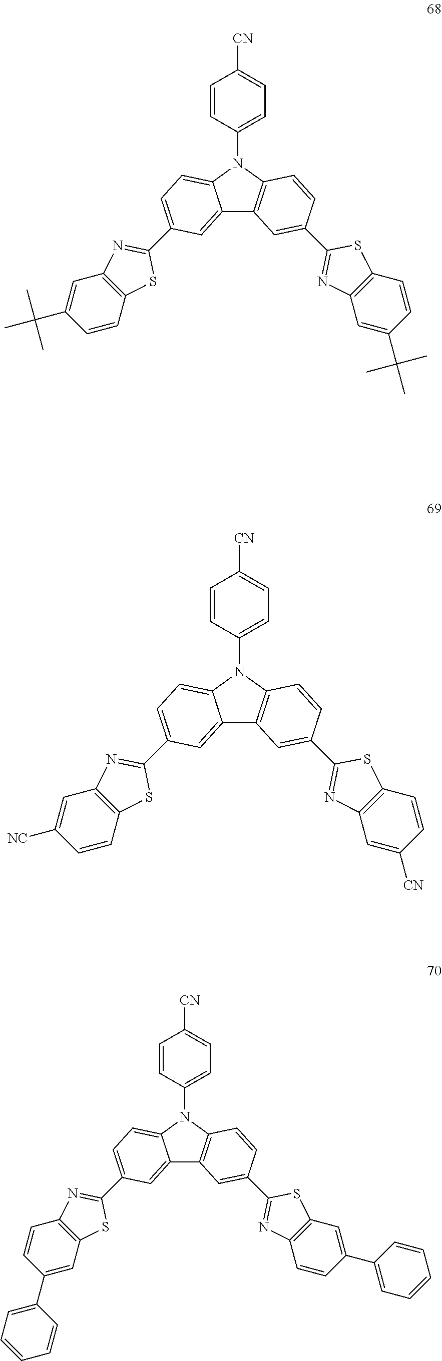 [SMILES: CC(C)(C)c1ccc2sc(-c3ccc4c(c3)c3cc(-c5nc6cc(C(C)(C)C)ccc6s5)ccc3n4-c3ccc(C#N)cc3)nc2c1 ; N#Cc1ccc(-n2c3ccc(-c4nc5cc(C#N)ccc5s4)cc3c3cc(-c4nc5cc(C#N)ccc5s4)ccc32)cc1 ; N#Cc1ccc(-n2c3ccc(-c4nc5ccc(-c6ccccc6)cc5s4)cc3c3cc(-c4nc5ccc(-c6ccccc6)cc5s4)ccc32)cc1]

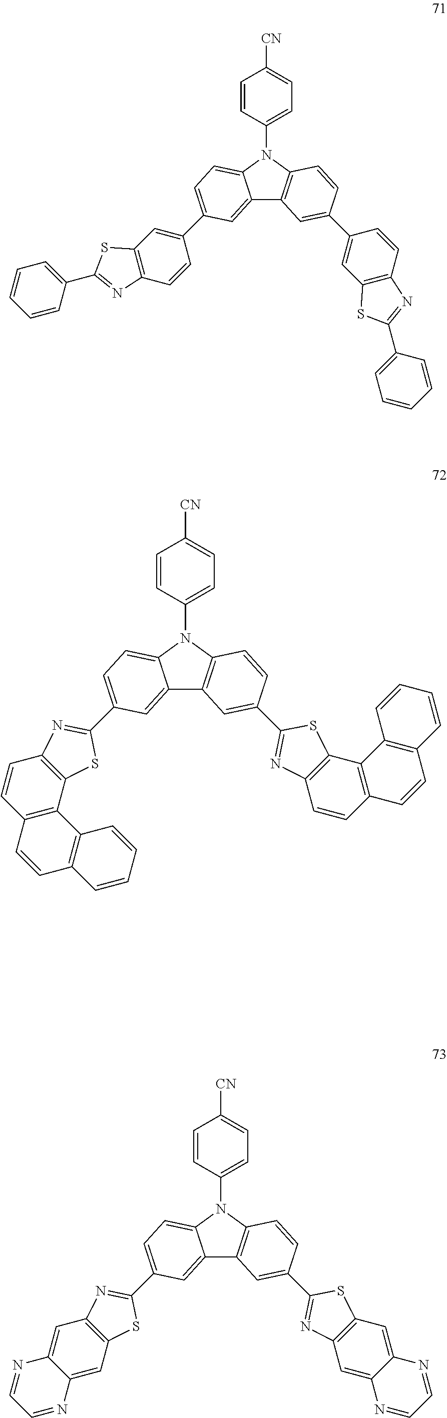 [SMILES: N#Cc1ccc(-n2c3ccc(-c4ccc5nc(-c6ccccc6)sc5c4)cc3c3cc(-c4ccc5nc(-c6ccccc6)sc5c4)ccc32)cc1 ; N#Cc1ccc(-n2c3ccc(-c4nc5ccc6ccc7ccccc7c6c5s4)cc3c3cc(-c4nc5ccc6ccc7ccccc7c6c5s4)ccc32)cc1 ; N#Cc1ccc(-n2c3ccc(-c4nc5cc6nccnc6cc5s4)cc3c3cc(-c4nc5cc6nccnc6cc5s4)ccc32)cc1]

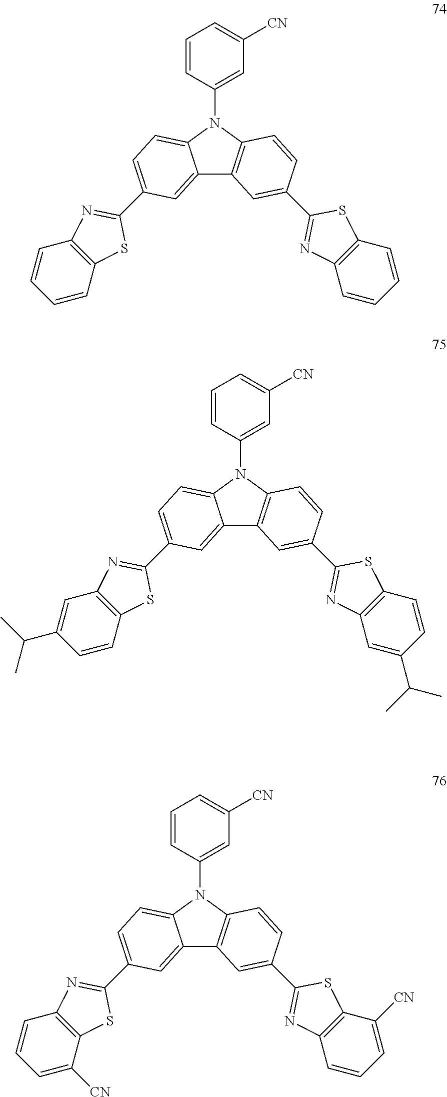 [SMILES: N#Cc1cccc(-n2c3ccc(-c4nc5ccccc5s4)cc3c3cc(-c4nc5ccccc5s4)ccc32)c1 ; CC(C)c1ccc2sc(-c3ccc4c(c3)c3cc(-c5nc6cc(C(C)C)ccc6s5)ccc3n4-c3cccc(C#N)c3)nc2c1 ; N#Cc1cccc(-n2c3ccc(-c4nc5cccc(C#N)c5s4)cc3c3cc(-c4nc5cccc(C#N)c5s4)ccc32)c1]

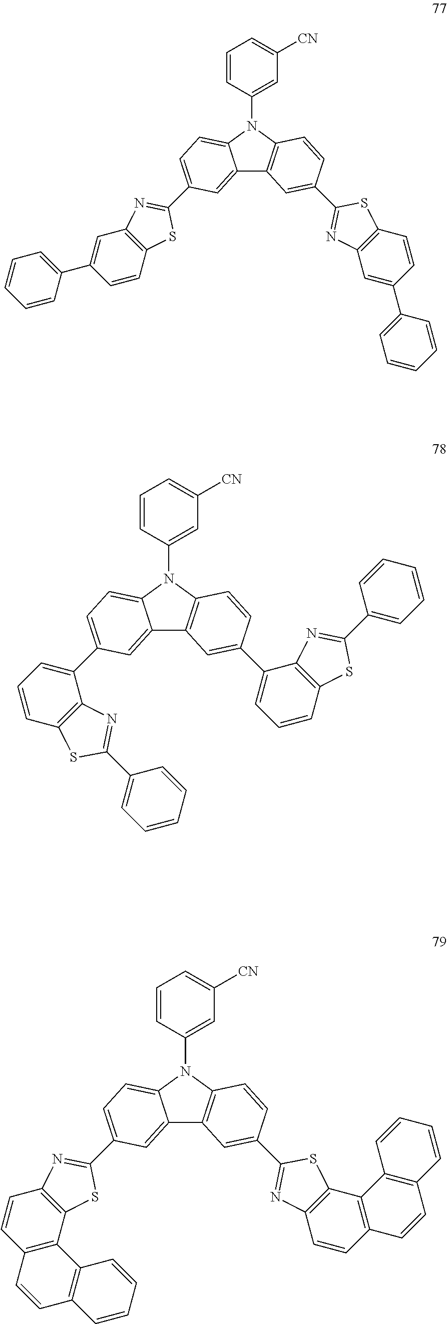 [SMILES: N#Cc1cccc(-n2c3ccc(-c4nc5cc(-c6ccccc6)ccc5s4)cc3c3cc(-c4nc5cc(-c6ccccc6)ccc5s4)ccc32)c1 ; N#Cc1cccc(-n2c3ccc(-c4cccc5sc(-c6ccccc6)nc45)cc3c3cc(-c4cccc5sc(-c6ccccc6)nc45)ccc32)c1 ; N#Cc1cccc(-n2c3ccc(-c4nc5ccc6ccc7ccccc7c6c5s4)cc3c3cc(-c4nc5ccc6ccc7ccccc7c6c5s4)ccc32)c1]

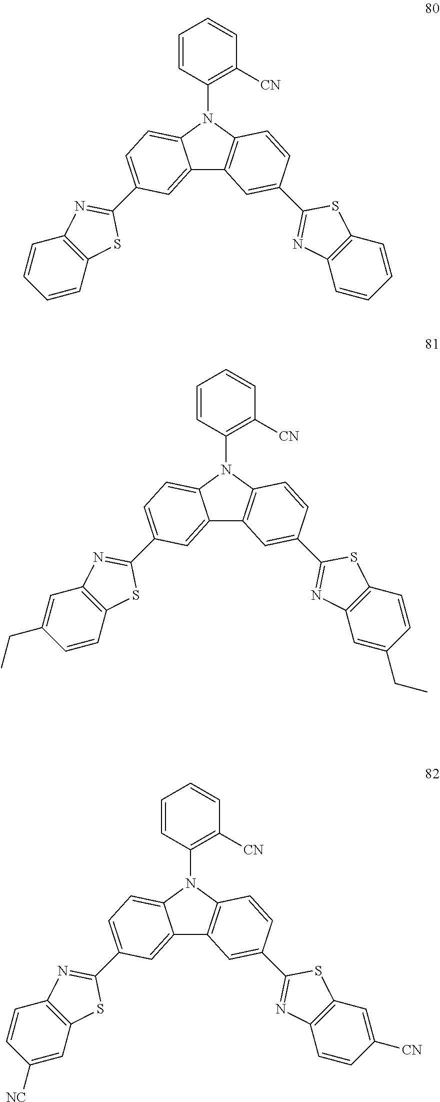 [SMILES: N#Cc1ccccc1-n1c2ccc(-c3nc4ccccc4s3)cc2c2cc(-c3nc4ccccc4s3)ccc21 ; CCc1ccc2sc(-c3ccc4c(c3)c3cc(-c5nc6cc(CC)ccc6s5)ccc3n4-c3ccccc3C#N)nc2c1 ; N#Cc1ccc2nc(-c3ccc4c(c3)c3cc(-c5nc6ccc(C#N)cc6s5)ccc3n4-c3ccccc3C#N)sc2c1]

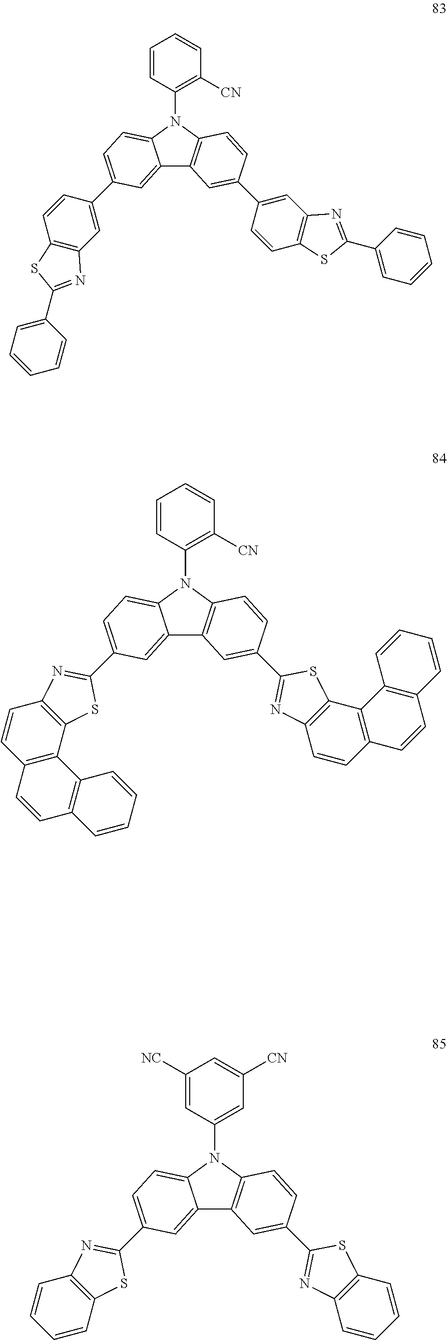 [SMILES: N#Cc1ccccc1-n1c2ccc(-c3ccc4sc(-c5ccccc5)nc4c3)cc2c2cc(-c3ccc4sc(-c5ccccc5)nc4c3)ccc21 ; N#Cc1ccccc1-n1c2ccc(-c3nc4ccc5ccc6ccccc6c5c4s3)cc2c2cc(-c3nc4ccc5ccc6ccccc6c5c4s3)ccc21 ; N#Cc1cc(C#N)cc(-n2c3ccc(-c4nc5ccccc5s4)cc3c3cc(-c4nc5ccccc5s4)ccc32)c1]

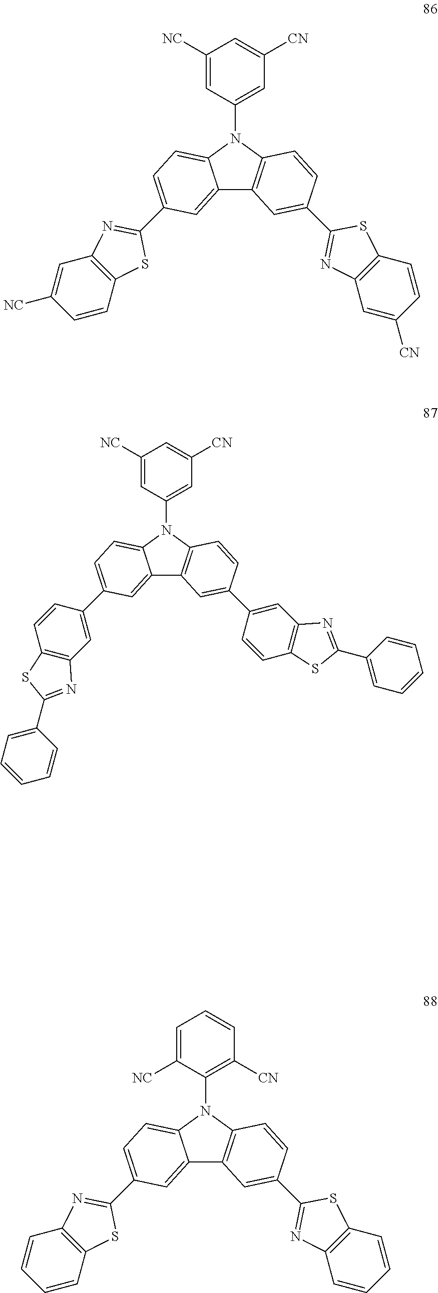 [SMILES: N#Cc1cc(C#N)cc(-n2c3ccc(-c4nc5cc(C#N)ccc5s4)cc3c3cc(-c4nc5cc(C#N)ccc5s4)ccc32)c1 ; N#Cc1cc(C#N)cc(-n2c3ccc(-c4ccc5sc(-c6ccccc6)nc5c4)cc3c3cc(-c4ccc5sc(-c6ccccc6)nc5c4)ccc32)c1 ; N#Cc1cccc(C#N)c1-n1c2ccc(-c3nc4ccccc4s3)cc2c2cc(-c3nc4ccccc4s3)ccc21]

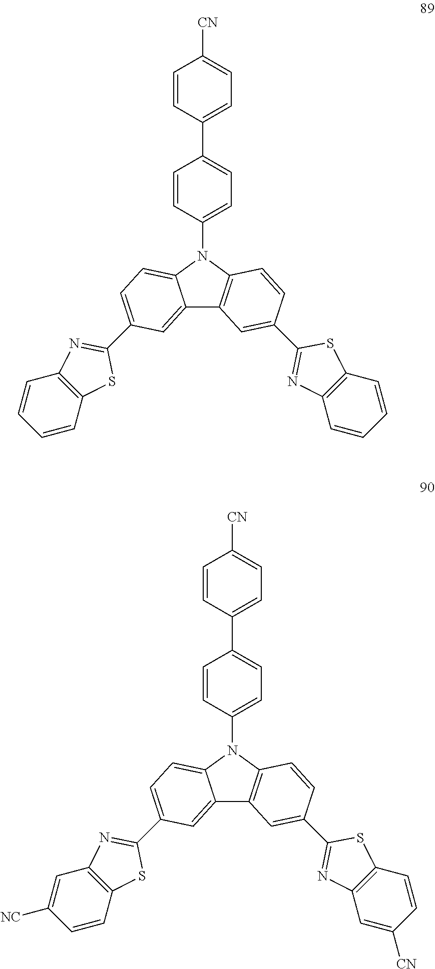 [SMILES: N#Cc1ccc(-c2ccc(-n3c4ccc(-c5nc6ccccc6s5)cc4c4cc(-c5nc6ccccc6s5)ccc43)cc2)cc1 ; N#Cc1ccc(-c2ccc(-n3c4ccc(-c5nc6cc(C#N)ccc6s5)cc4c4cc(-c5nc6cc(C#N)ccc6s5)ccc43)cc2)cc1]

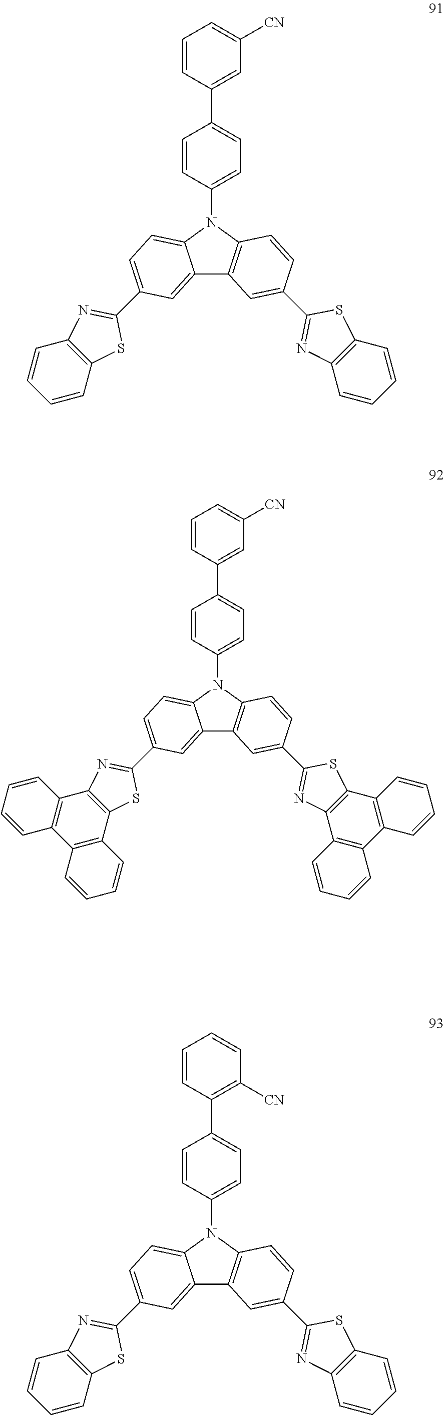 [SMILES: N#Cc1cccc(-c2ccc(-n3c4ccc(-c5nc6ccccc6s5)cc4c4cc(-c5nc6ccccc6s5)ccc43)cc2)c1 ; N#Cc1cccc(-c2ccc(-n3c4ccc(-c5nc6c7ccccc7c7ccccc7c6s5)cc4c4cc(-c5nc6c7ccccc7c7ccccc7c6s5)ccc43)cc2)c1 ; N#Cc1ccccc1-c1ccc(-n2c3ccc(-c4nc5ccccc5s4)cc3c3cc(-c4nc5ccccc5s4)ccc32)cc1]

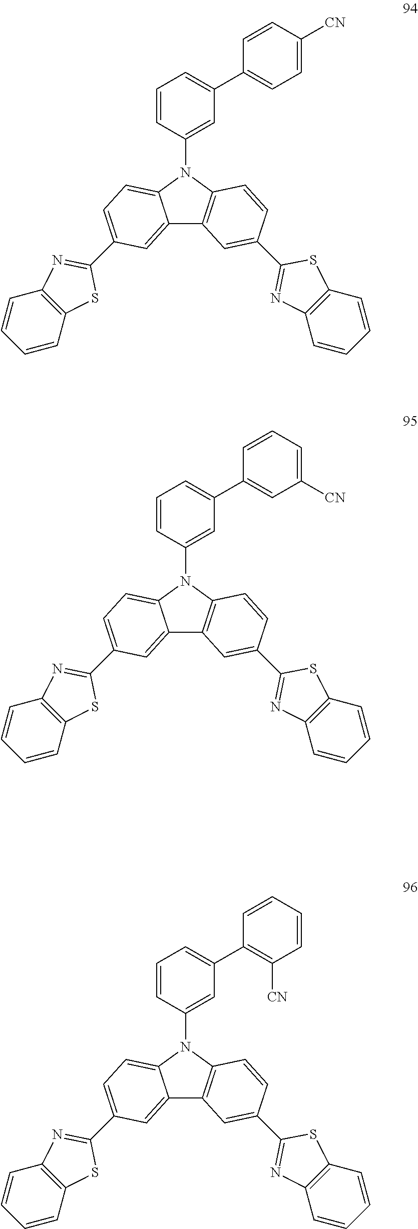 [SMILES: N#Cc1ccc(-c2cccc(-n3c4ccc(-c5nc6ccccc6s5)cc4c4cc(-c5nc6ccccc6s5)ccc43)c2)cc1 ; N#Cc1cccc(-c2cccc(-n3c4ccc(-c5nc6ccccc6s5)cc4c4cc(-c5nc6ccccc6s5)ccc43)c2)c1 ; N#Cc1ccccc1-c1cccc(-n2c3ccc(-c4nc5ccccc5s4)cc3c3cc(-c4nc5ccccc5s4)ccc32)c1]

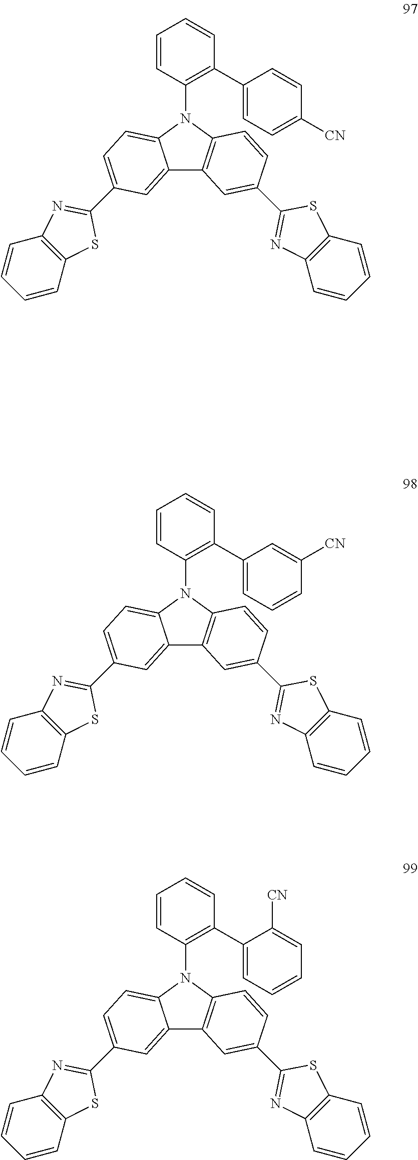 [SMILES: N#Cc1ccc(-c2ccccc2-n2c3ccc(-c4nc5ccccc5s4)cc3c3cc(-c4nc5ccccc5s4)ccc32)cc1 ; N#Cc1cccc(-c2ccccc2-n2c3ccc(-c4nc5ccccc5s4)cc3c3cc(-c4nc5ccccc5s4)ccc32)c1 ; N#Cc1ccccc1-c1ccccc1-n1c2ccc(-c3nc4ccccc4s3)cc2c2cc(-c3nc4ccccc4s3)ccc21]

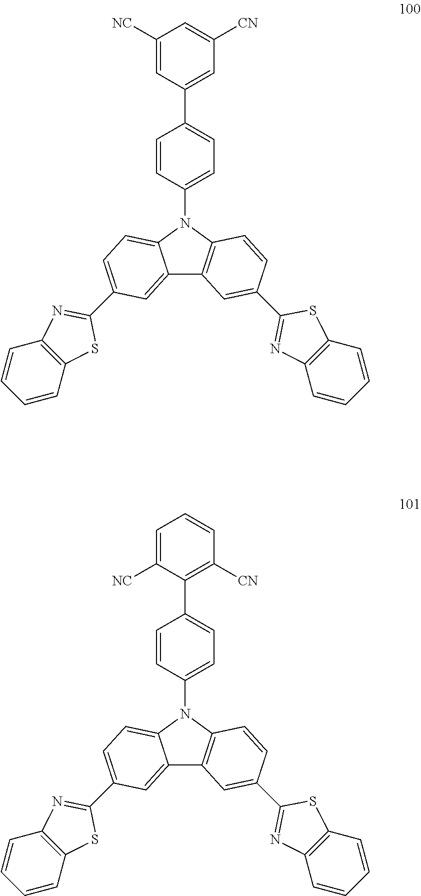 [SMILES: N#Cc1cc(C#N)cc(-c2ccc(-n3c4ccc(-c5nc6ccccc6s5)cc4c4cc(-c5nc6ccccc6s5)ccc43)cc2)c1 ; N#Cc1cccc(C#N)c1-c1ccc(-n2c3ccc(-c4nc5ccccc5s4)cc3c3cc(-c4nc5ccccc5s4)ccc32)cc1]

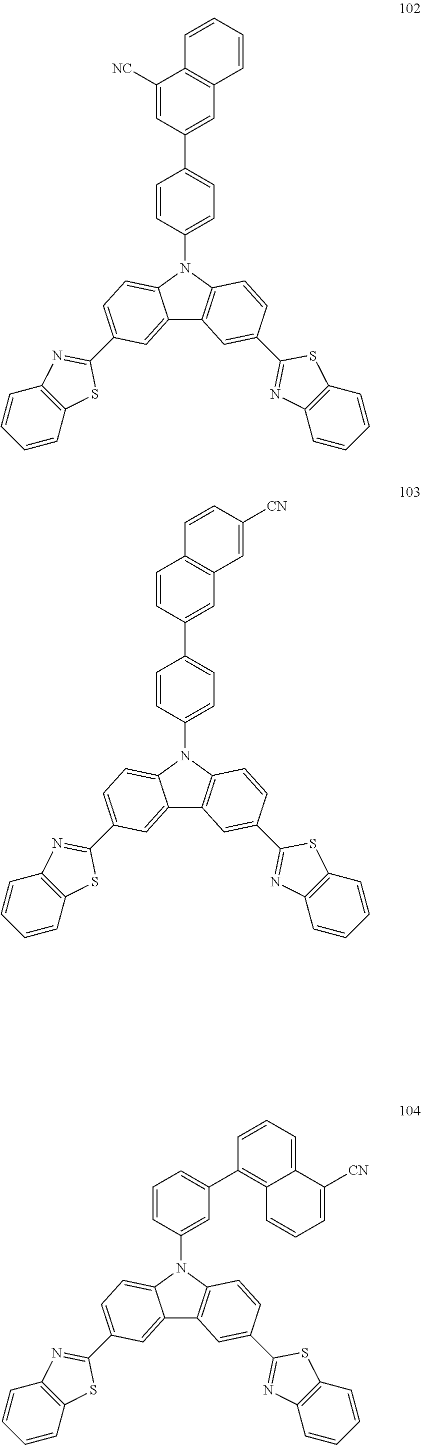 [SMILES: N#Cc1cc(-c2ccc(-n3c4ccc(-c5nc6ccccc6s5)cc4c4cc(-c5nc6ccccc6s5)ccc43)cc2)cc2ccccc12 ; N#Cc1ccc2ccc(-c3ccc(-n4c5ccc(-c6nc7ccccc7s6)cc5c5cc(-c6nc7ccccc7s6)ccc54)cc3)cc2c1 ; N#Cc1cccc2c(-c3cccc(-n4c5ccc(-c6nc7ccccc7s6)cc5c5cc(-c6nc7ccccc7s6)ccc54)c3)cccc12]

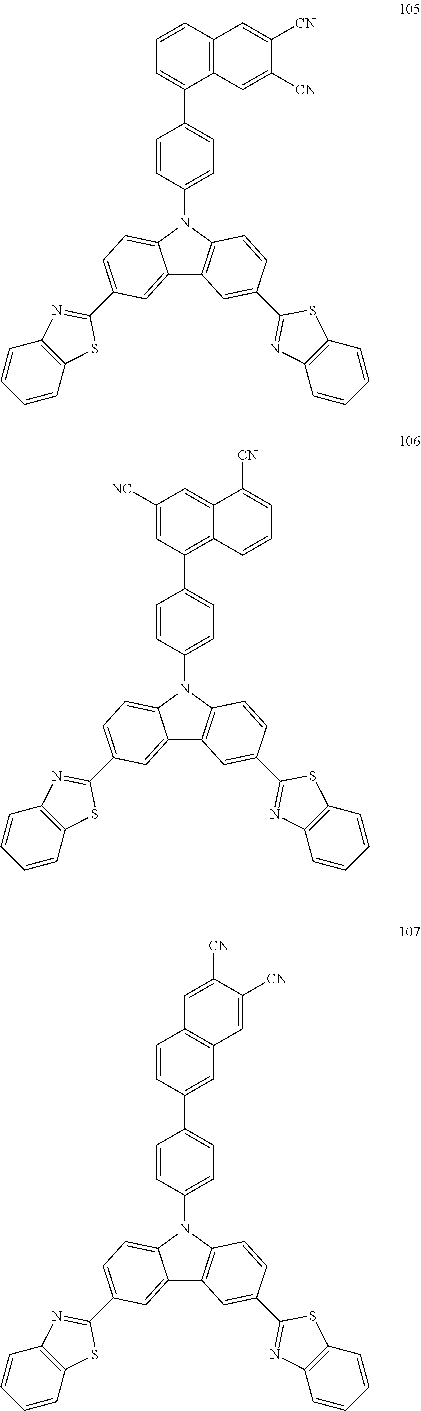 [SMILES: N#Cc1cc2cccc(-c3ccc(-n4c5ccc(-c6nc7ccccc7s6)cc5c5cc(-c6nc7ccccc7s6)ccc54)cc3)c2cc1C#N ; N#Cc1cc(-c2ccc(-n3c4ccc(-c5nc6ccccc6s5)cc4c4cc(-c5nc6ccccc6s5)ccc43)cc2)c2cccc(C#N)c2c1 ; N#Cc1cc2ccc(-c3ccc(-n4c5ccc(-c6nc7ccccc7s6)cc5c5cc(-c6nc7ccccc7s6)ccc54)cc3)cc2cc1C#N]

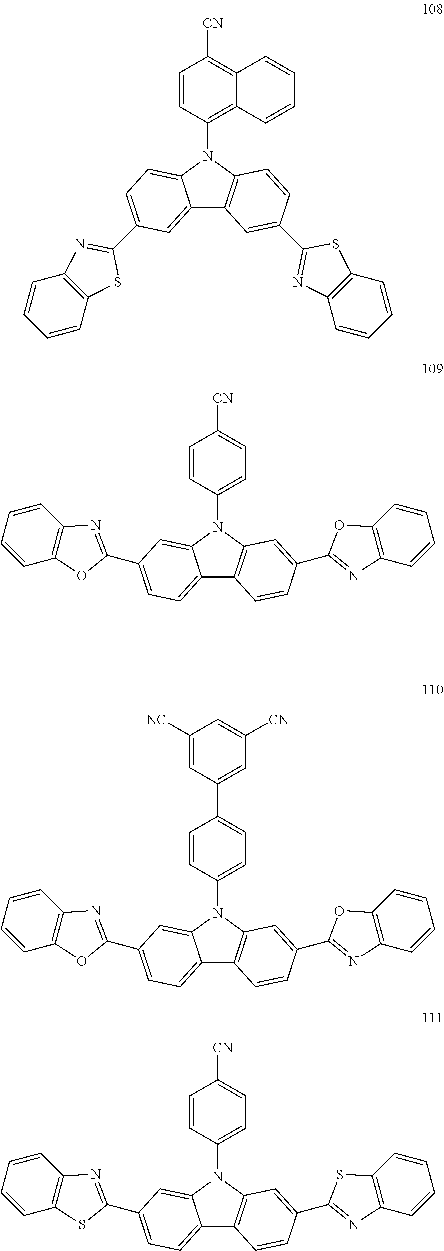 [SMILES: N#Cc1ccc(-n2c3ccc(-c4nc5ccccc5s4)cc3c3cc(-c4nc5ccccc5s4)ccc32)c2ccccc12 ; N#Cc1ccc(-n2c3cc(-c4nc5ccccc5o4)ccc3c3ccc(-c4nc5ccccc5o4)cc32)cc1 ; N#Cc1cc(C#N)cc(-c2ccc(-n3c4cc(-c5nc6ccccc6o5)ccc4c4ccc(-c5nc6ccccc6o5)cc43)cc2)c1 ; N#Cc1ccc(-n2c3cc(-c4nc5ccccc5s4)ccc3c3ccc(-c4nc5ccccc5s4)cc32)cc1]

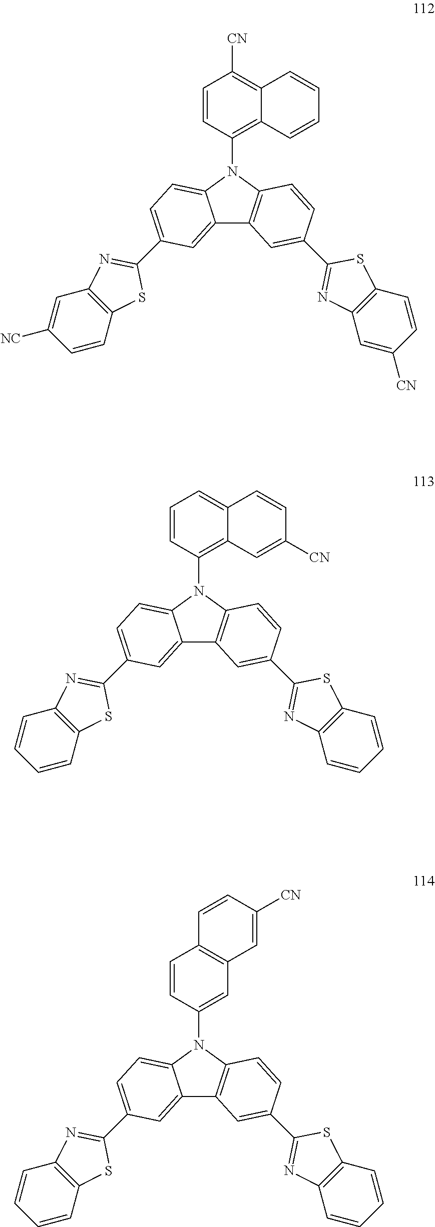 [SMILES: N#Cc1ccc2sc(-c3ccc4c(c3)c3cc(-c5nc6cc(C#N)ccc6s5)ccc3n4-c3ccc(C#N)c4ccccc34)nc2c1 ; N#Cc1ccc2cccc(-n3c4ccc(-c5nc6ccccc6s5)cc4c4cc(-c5nc6ccccc6s5)ccc43)c2c1 ; N#Cc1ccc2ccc(-n3c4ccc(-c5nc6ccccc6s5)cc4c4cc(-c5nc6ccccc6s5)ccc43)cc2c1]

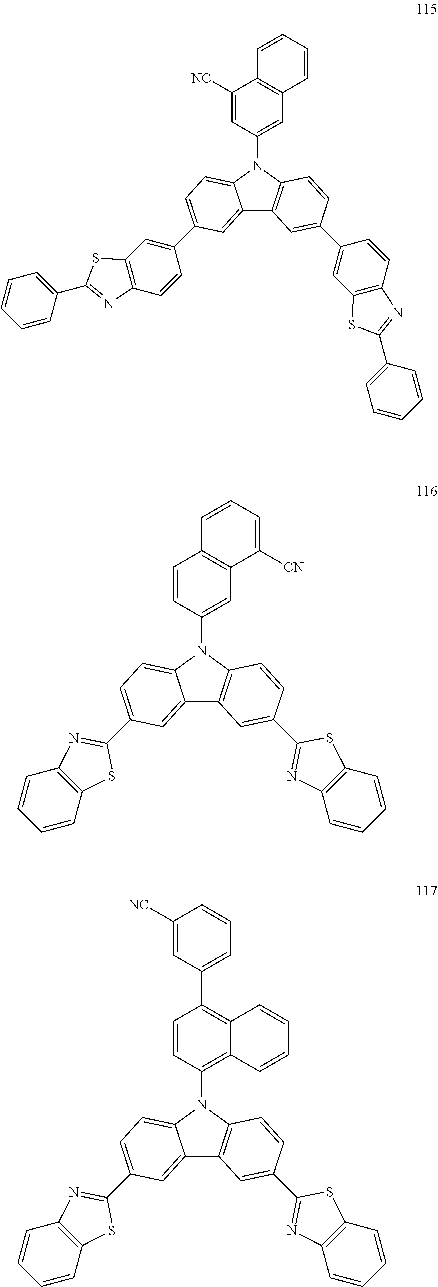 [SMILES: N#Cc1cc(-n2c3ccc(-c4ccc5nc(-c6ccccc6)sc5c4)cc3c3cc(-c4ccc5nc(-c6ccccc6)sc5c4)ccc32)cc2ccccc12 ; N#Cc1cccc2ccc(-n3c4ccc(-c5nc6ccccc6s5)cc4c4cc(-c5nc6ccccc6s5)ccc43)cc12 ; N#Cc1cccc(-c2ccc(-n3c4ccc(-c5nc6ccccc6s5)cc4c4cc(-c5nc6ccccc6s5)ccc43)c3ccccc23)c1]

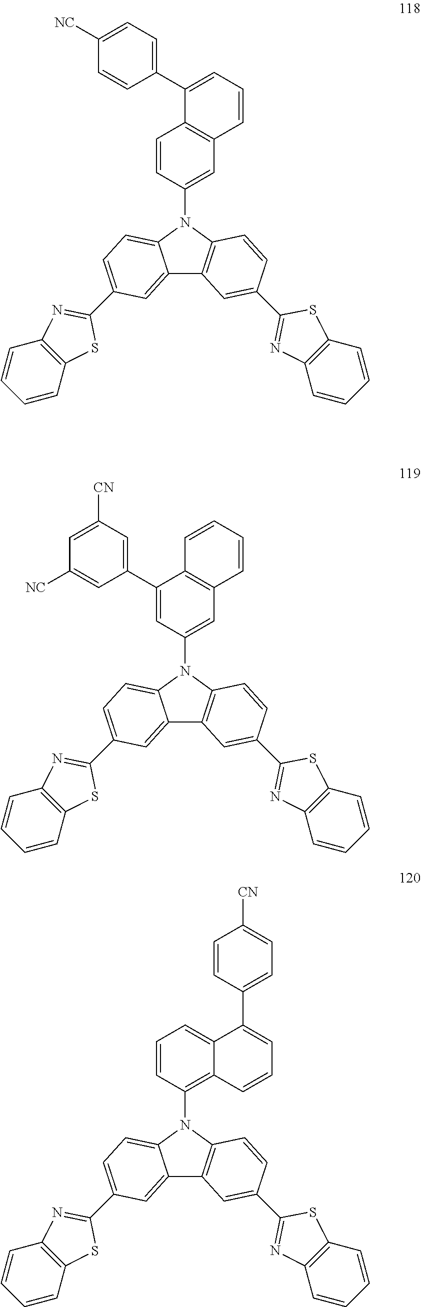 [SMILES: N#Cc1ccc(-c2cccc3cc(-n4c5ccc(-c6nc7ccccc7s6)cc5c5cc(-c6nc7ccccc7s6)ccc54)ccc23)cc1 ; N#Cc1cc(C#N)cc(-c2cc(-n3c4ccc(-c5nc6ccccc6s5)cc4c4cc(-c5nc6ccccc6s5)ccc43)cc3ccccc23)c1 ; N#Cc1ccc(-c2cccc3c(-n4c5ccc(-c6nc7ccccc7s6)cc5c5cc(-c6nc7ccccc7s6)ccc54)cccc23)cc1]

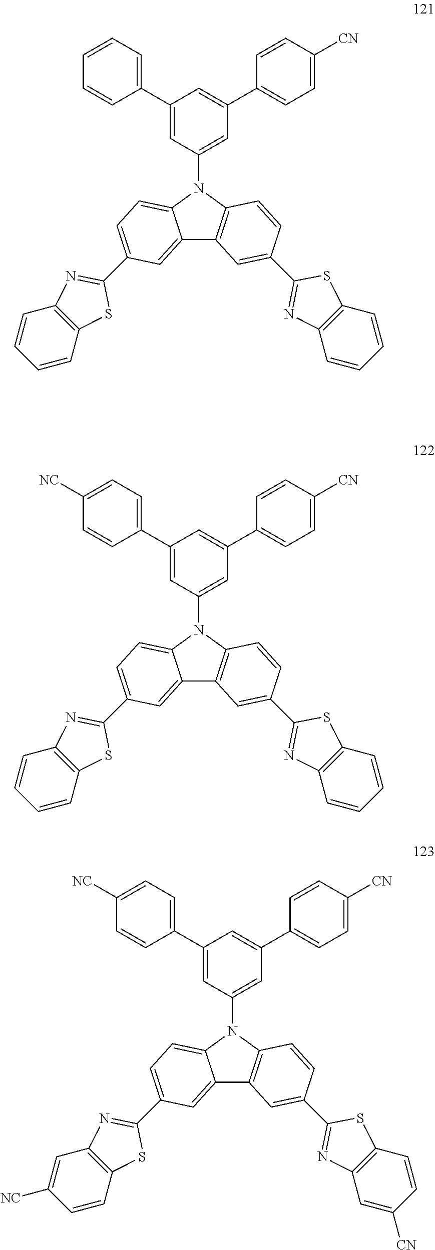 [SMILES: N#Cc1ccc(-c2cc(-c3ccccc3)cc(-n3c4ccc(-c5nc6ccccc6s5)cc4c4cc(-c5nc6ccccc6s5)ccc43)c2)cc1 ; N#Cc1ccc(-c2cc(-c3ccc(C#N)cc3)cc(-n3c4ccc(-c5nc6ccccc6s5)cc4c4cc(-c5nc6ccccc6s5)ccc43)c2)cc1 ; N#Cc1ccc(-c2cc(-c3ccc(C#N)cc3)cc(-n3c4ccc(-c5nc6cc(C#N)ccc6s5)cc4c4cc(-c5nc6cc(C#N)ccc6s5)ccc43)c2)cc1]

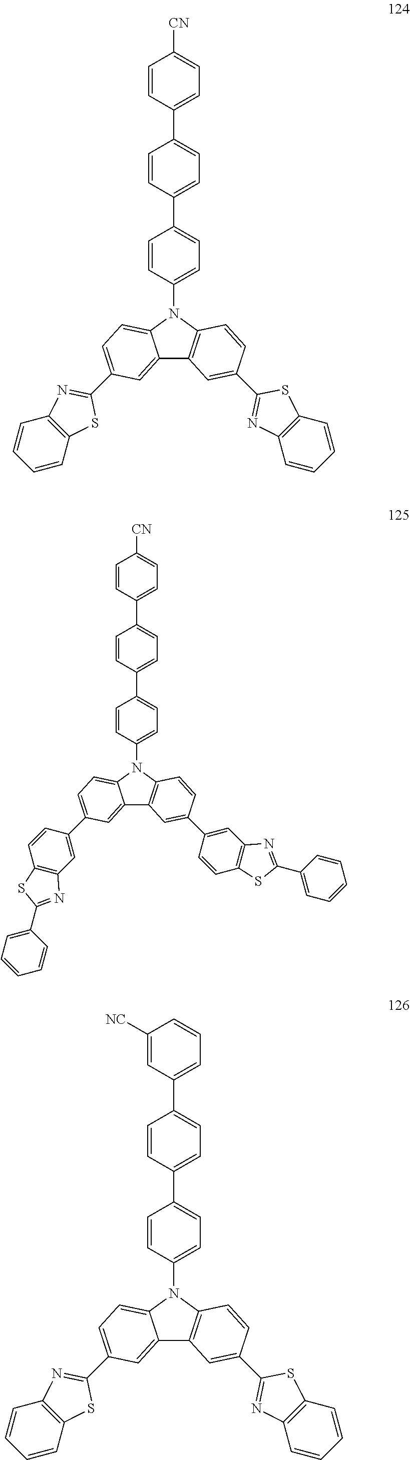 [SMILES: N#Cc1ccc(-c2ccc(-c3ccc(-n4c5ccc(-c6nc7ccccc7s6)cc5c5cc(-c6nc7ccccc7s6)ccc54)cc3)cc2)cc1 ; N#Cc1ccc(-c2ccc(-c3ccc(-n4c5ccc(-c6ccc7sc(-c8ccccc8)nc7c6)cc5c5cc(-c6ccc7sc(-c8ccccc8)nc7c6)ccc54)cc3)cc2)cc1 ; N#Cc1cccc(-c2ccc(-c3ccc(-n4c5ccc(-c6nc7ccccc7s6)cc5c5cc(-c6nc7ccccc7s6)ccc54)cc3)cc2)c1]

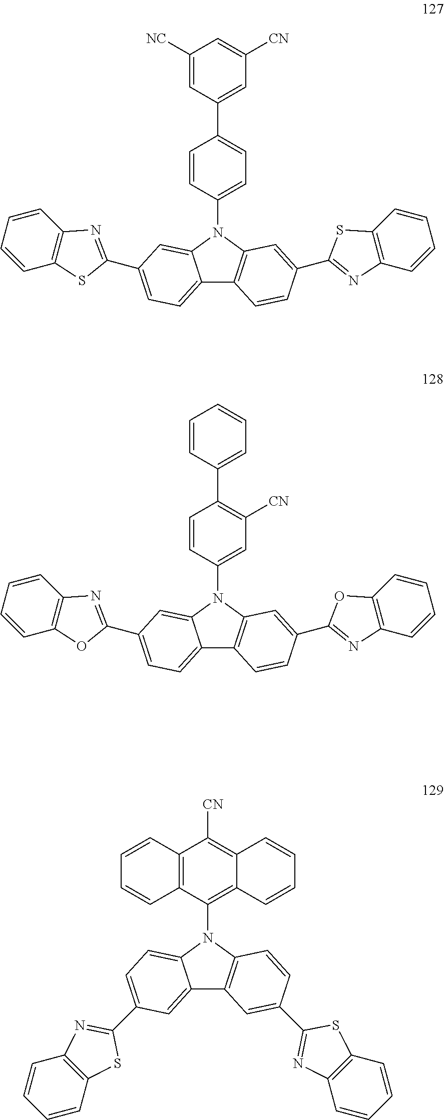 [SMILES: N#Cc1cc(C#N)cc(-c2ccc(-n3c4cc(-c5nc6ccccc6s5)ccc4c4ccc(-c5nc6ccccc6s5)cc43)cc2)c1 ; N#Cc1cc(-n2c3cc(-c4nc5ccccc5o4)ccc3c3ccc(-c4nc5ccccc5o4)cc32)ccc1-c1ccccc1 ; N#Cc1c2ccccc2c(-n2c3ccc(-c4nc5ccccc5s4)cc3c3cc(-c4nc5ccccc5s4)ccc32)c2ccccc12]

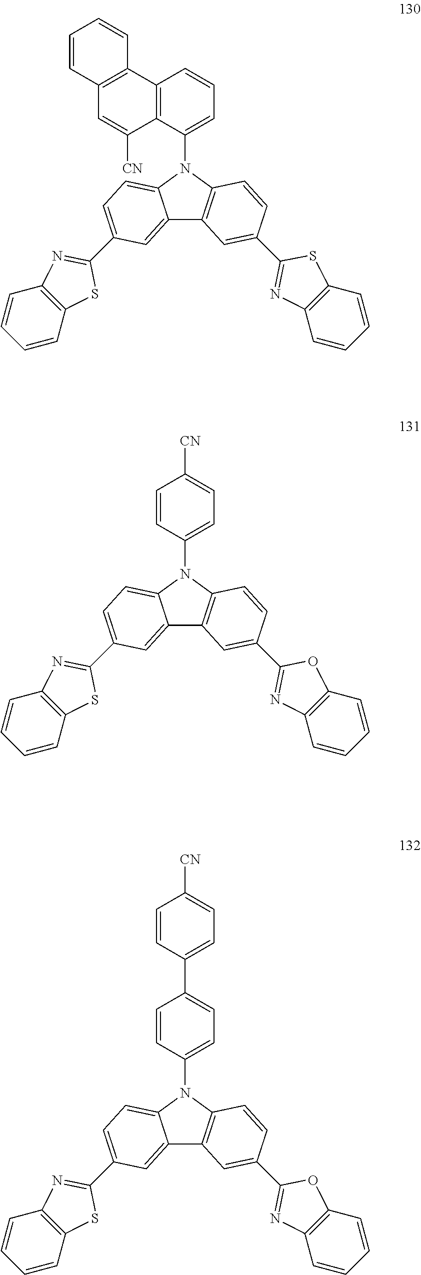 [SMILES: N#Cc1cc2ccccc2c2cccc(-n3c4ccc(-c5nc6ccccc6s5)cc4c4cc(-c5nc6ccccc6s5)ccc43)c12 ; N#Cc1ccc(-n2c3ccc(-c4nc5ccccc5o4)cc3c3cc(-c4nc5ccccc5s4)ccc32)cc1 ; N#Cc1ccc(-c2ccc(-n3c4ccc(-c5nc6ccccc6o5)cc4c4cc(-c5nc6ccccc6s5)ccc43)cc2)cc1]

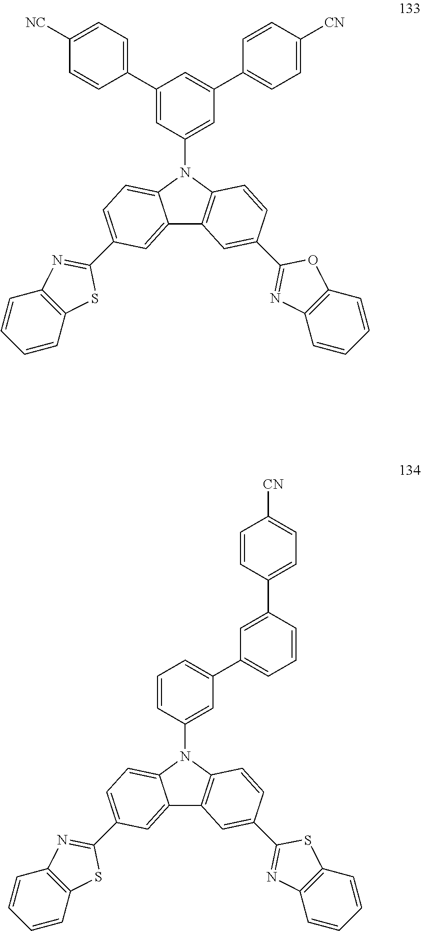 [SMILES: N#Cc1ccc(-c2cc(-c3ccc(C#N)cc3)cc(-n3c4ccc(-c5nc6ccccc6o5)cc4c4cc(-c5nc6ccccc6s5)ccc43)c2)cc1 ; N#Cc1ccc(-c2cccc(-c3cccc(-n4c5ccc(-c6nc7ccccc7s6)cc5c5cc(-c6nc7ccccc7s6)ccc54)c3)c2)cc1]

5. An organic light emitting device comprising a first electrode, a second electrode, and an organic material layer having one or more layers disposed between the first electrode and the second electrode, wherein one or more layers of the organic layer comprise the organic compound of [Formula 1] according to claim 1.

6. The organic light emitting device of claim 5, wherein the organic layer comprises one or more layers selected from among a hole injection layer, a hole transport layer, a layer which simultaneously injects and transports holes, an electron transport layer, an electron injection layer, a layer which simultaneously transports and injects electrons, an electron blocking layer, a hole blocking layer, and a light emitting device, and
one or more layers of the layers comprise the organic compound represented by Formula I above.

7. The organic light emitting device of claim 5, wherein any one of the electron transport layer, the electron injection layer and the layer which simultaneously transports and injects electrons comprises the organic compound represented by Formula I above.

8. The organic light emitting device of claim 5, further comprising a light efficiency improving layer (capping layer) formed on at least one side opposite to the organic layer at the top or bottom of the first electrode and the second electrode,
wherein the light efficiency improving layer comprises the organic compound represented by Formula I above.

9. The organic light emitting device of claim 8, wherein the light efficiency improving layer is formed on at least one of the bottom of the first electrode and the top of the second electrode.
